SEQUENCE LISTING (18 sequences)

SEQ ID NO: 1 - DNA, 1242 bp, Artificial Sequence
  Description of Artificial Sequence Synthetic polynucleotide
atgccgagcagccatcatcatcatcatcacgattacgatatcccaacgaccgaaaacctttacttccagggatccatgaaaacgattacgctgtacctcgatccggccagcctcccagcgctgaaccaactgatggacttcacgcagaacaacgaggacaaaacgcacccgcgcatcttcggtctgagtcgctttaaaatcccggataacattatcacccagtaccagaacatccactttgtggaactgaaggacaatcgcccgaccgaagcgctgttcaccatcctcgatcagtacccgggcaacattgagctgaatatccatctgaatattgcgcatagcgtgcagctgatccgcccgattctggcctaccgcttcaaacatctggatcgcgttagcatccagcagctgaacctctacgatgatggcagcatggagtacgttgatctggagaaggaggagaataaggatatcagtgcggagatcaaacaagccgagaagcagctgagccactatctgctgaccggcaagatcaagttcgataacccgaccatcgcccgctacgtttggcagagcgcctttccggtgaaataccactttctgagcaccgactactttgagaaggcggaatttctgcagccgctgaaggaatatctggcggagaactaccagaagatggattggaccgcgtaccagcaactgaccccagagcaacaagccttttatctcacgctggtgggtttcaacgacgaggtgaaacagagtctggaagtgcagcaagccaagttcatcttcacgggtaccaccacgtgggaaggtaacaccgacgttcgcgaatactacgcgcagcagcagctcaatctgctgaatcacttcacgcaagccgaaggcgatctgttcatcggtgaccactacaaaatttacttcaaaggtcacccgcgcggcggcgagatcaacgattatattctgaacaacgcgaaaaatatcacgaacatcccggccaacatcagctttgaagtgctgatgatgacgggtctgctgccagataaggttggcggtgttgccagcagtctgtacttcagcctcccaaaagaaaaaatcagccatatcatctttacgagtaataaacaagtgaaaagcaaggaggatgcgctgaacaatccgtacgtgaaggtgatgcgccgcctcggcatcatcgatgagagccaagttatcttctgggacagtctgaagcagctgtaa

SEQ ID NO: 2 - DNA, 762 bp, Artificial Sequence
  Description of Artificial Sequence Synthetic polynucleotide
atgccgagcagccatcatcatcatcatcacgattacgatatcccaacgaccgaaaacctttacttccagggatccatggagaaacagaatatcgcggtgattctggcccgccagaatagcaaaggtctgccgctgaagaatctgcgcaaaatgaacggcatcagtctgctgggccataccatcaatgccgcgatcagcagcaagtgcttcgaccgcattatcgttagcaccgatggcggtctgattgccgaagaagcgaaaaacttcggcgtggaggttgttctgcgcccagcggaactcgcgagtgataccgccagcagcattagcggcgttattcacgcgctggagacgattggcagcaatagcggcacggtgacgctgctgcaaccaaccagcccgctgcgtaccggtgcccatattcgcgaggccttcagcctcttcgacgagaagatcaaaggcagcgtggttagcgcgtgcccgatggaacatcacccgctgaaaacgctgctgcagatcaacaatggcgagtacgccccaatgcgccatctgagcgatctggagcagccgcgtcagcaactgccacaagccttccgcccgaacggcgccatctacatcaatgacaccgcgagtctgatcgccaacaactgcttcttcatcgcgccgacgaagctgtacatcatgagccatcaagatagcatcgacatcgataccgagctggatctgcagcaagccgagaacattctgaaccacaaagagagctaa

SEQ ID NO: 3 - DNA, 1629 bp, Artificial Sequence
  Description of Artificial Sequence Synthetic polynucleotide
atggcaagcagccatcatcatcatcatcatggcagcagcgtgagcaaaggcgaagaactgtttaccggcgtggtgccgattctggtggaactggatggcgatgtgaacggccataaatttagcgtgcgcggcgaaggcgaaggcgatgcgaccaacggcaaactgaccctgaaatttatttgcaccaccggcaaactgccggtgccgtggccgaccctggtgaccaccctgacctatggcgtgcagtgctttagccgctatccggatcatatgaaacagcatgatttttttaaaagcgcgatgccggaaggctatgtgcaagaacgcaccattagctttaaagatgatggcacctataaaacccgcgcggaagtgaaatttgaaggcgataccctggtgaaccgcattgaactgaaaggcattgattttaaagaagatggcaacattctgggccataaactggaatataactttaacagccataacgtgtatattaccgcggataaacagaaaaacggcattaaagcgaactttaaaattcgccataacgtggaagatggcagcgtgcagctggcggatcattatcagcagaacaccccgattggcgatggcccggtgctgctgccggataaccattatctgagcacgcagagcaaactgagcaaagatccgaacgaaaaacgcgatcatatggtgctgctggaatttgtgaccgcggcgggcattaccctgggcatggatgaactgtataaactgggatccgcgagcatggattttaaaattgtgcaagtgcatggcggcctgggcaatcagatgtttcagtatgcgtttgcgaaaagcctgcagacccatctgaacattccggtgctgctggataccacctggtttgattatggcaaccgcgaactgggcctgcatctgtttccgattgatctgcagtgcgcgagcgcgcagcagattgctgcggcgcatatgcagaacctgccgcgcctggtccgcggagcgcttcgccgcatgggcctgggccgcgtgagcaaagaaattgtgtttgaatatatgccggaactgtttgaaccgagccgcattgcgtattttcatggctattttcaagatccgcgctattttgaagatattagcccgctgattaaacagacctttaccctgccgcatccgaccgaacatgcggaacagtatagccgcaaactgagtcagattctggcggcgaaaaacagcgtgtttgtgcatattcgccgcggcgattatatgcgcctgggctggcagctggatattagctatcagctgcgcgcgattgcgtatatggcgaaacgcgtacagaacctggaactgtttctgttttgcgaagatctggaatttgttcagaacctggatctgggctatccgtttgtggatatgaccacccgcgatggcgcggcgcattgggatatgatgctgatgcagagctgcaaacatggcattattaccaacagcacctatagctggtgggcggcgtatctgattaaaaacccggaaaaaattattattggcccgagccattggatttatggcaacgaaaacattctgtgcaaagattgggtgaaaattgaaagtcagtttgaaaccaaaagctaa

SEQ ID NO: 4 - DNA, 1665 bp, Artificial Sequence
  Description of Artificial Sequence Synthetic polynucleotide
atgaaatcttcccaccatcaccatcaccatggttcttctgtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctccttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggcgaacttcaagatccgccacaacgtcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagctcgggtccgacctagtaccacgtggatcgatgattatcgtacatttgtgcggaggattggggaaccaaatgtttcaatatgcagctggactggcagcggcgcatcgtatcggatctgaagtaaaatttgacactcattggtttgatgccacgtgtctgcaccaaggccttgagttgcgtcgcgtattcggtttagagctgcccgaaccttcctcaaaggatttgcgtaaggtactgggggcgtgcgttcatccggccgttcgccgcttactggctggccactttcttcatggcttacgtccgaagagccttgtcattcagccgcattttcattattggactgggtttgagcacctgccggacaatgtatatttagagggctactggcaatcggagcgctatttctcgaacattgctgatattattcgccaacagtttcgctttgtagaaccgttggaccctcataacgctgctttgatggacgaaatgcagtccggtgtcagtgtgagtctgcatatccgccgtggtgactacttcaacaatccccagatgcgccgtgtacatggcgttgatctgtctgaatattatccggctgctgtcgccacgatgattgagaagacaaatgccgagcgtttttacgtgttcagcgatgatccccagtgggtcttagagcatctgaagttgccggtatcgtacaccgttgtagatcataatcgtggagctgcctcttatcgtgatatgcaattgatgtccgcttgtcgccatcacattatcgctaatagcaccttctcgtggtggggggcatggctgaatcctcgccctgacaaagtagtgattgcgccccgccattggtttaacgtggacgtgtttgatactcgtgatttgtactgtcctggatggattgttctgtaa

SEQ ID NO: 5 - DNA, 2925 bp, Artificial Sequence
  Description of Artificial Sequence Synthetic polynucleotide
atgccgagcagccatcatcatcatcatcacgattacgatatcccaacgaccgaaaacctttacttccagggatccatgcaaaaactgctgagcctgccgagcaatctggttcaaagcttccacgaactggagcgtgttaatcgcaccgactggttctgcaccagcgatccggttggcaagaaactgggtagcggtggcggtaccagctggctgctggaggaatgctacaacgagtatagcgacggcgcgaccttcggtgaatggctggagaaggaaaaacgtatcctgctgcatgcgggcggtcagagccgtcgtctgccgggctacgcgccgagcggtaaaattctgaccccggtgccggtttttcgttgggagcgtggccagcacctgggtcaaaacctgctgagcctgcagctgccgctgtatgaaaagatcatgagcctggcgccggataaactgcacaccctgatcgcgagcggtgacgtttacattcgtagcgagaagccgctgcaaagcattccggaagcggatgtggtttgctatggtctgtgggttgacccgagcctggcgacccaccatggtgtgttcgcgagcgatcgtaagcacccggagcagctggactttatgctgcaaaaaccgagcctggcggagctggaaagcctgagcaagacccacctgttcctgatggatatcggtatttggctgctgagcgaccgtgcggtggagatcctgatgaagcgtagccacaaagaaagcagcgaggaactgaaatactatgacctgtacagcgattttggtctggcgctgggtacccacccgcgtatcgaagatgaggaagtgaacaccctgagcgttgcgattctgccgctgccgggcggtgagttttaccactatggtaccagcaaagaactgatcagcagcaccctgagcgtgcagaacaaggtttacgaccaacgtcgtattatgcaccgtaaggttaaaccgaacccggcgatgttcgtgcagaacgcggtggttcgtatcccgctgtgcgcggagaacgcggatctgtggatcgaaaacagccacattggcccgaagtggaaaattgcgagccgtcacatcattaccggtgttccggagaacgactggagcctggcggtgccggcgggcgtgtgcgttgatgtggttccgatgggcgacaaaggtttcgttgcgcgtccgtacggcctggacgatgtgtttaagggtgacctgcgtgatagcaaaaccaccctgaccggcatcccgttcggtgaatggatgagcaagcgtggcctgagctataccgatctgaaaggtcgtaccgacgatctgcaagctgtgagcgtttttccgatggtgaacagcgttgaggaactgggtctggttctgcgttggatgctgagcgagccggaactggaggaaggcaaaaacatctggctgcgtagcgagcacttcagcgcggacgaaattagcgcgggtgcgaacctgaagcgtctgtacgcgcagcgtgaggaatttcgtaagggcaactggaaagcgctggcggtgaaccacgagaagagcgttttctatcaactggacctggcggatgcggcggaagattttgtgcgtctgggtctggacatgccggagctgctgccggaagatgcgctgcagatgagccgtatccacaaccgtatgctgcgtgcgcgtattctgaagctggacggtaaagattaccgtccggaggaacaagcggcgttcgacctgctgcgtgatggcctgctggacggtatcagcaaccgtaagagcaccccgaaactggatgtgtatagcgaccagatcgtttggggtcgtagcccggtgcgtattgatatggcgggcggttggaccgacaccccgccgtacagcctgtatagcggcggtaacgtggttaacctggcgatcgagctgaacggtcagccgccgctgcaagtgtacgttaagccgtgcaaagattttcacatcgttctgcgtagcattgacatgggcgcgatggagattgtgagcaccttcgatgaactgcaggactacaagaaaatcggtagcccgtttagcattccgaaagcggcgctgagcctggcgggttttgcgccggcgtttagcgcggttagctatgcgagcctggaggaacaactgaaggatttcggtgcgggtattgaggtgaccctgctggcggcgattccggcgggtagcggtctgggtaccagcagcatcctggcgagcaccgttctgggtgcgattaacgatttttgcggtctggcgtgggacaaaaacgagatctgccagcgtaccctggtgctggaacaactgctgaccaccggcggtggctggcaggaccaatacggtggcgtgctgcagggcgttaagctgctgcaaaccgaagcgggttttgcgcagagcccgctggttcgttggctgccggatcacctgtttacccacccggaatataaagactgccacctgctgtactataccggcatcacccgtaccgcgaagggtatcctggcggagattgtgagcagcatgttcctgaacagcagcctgcacctgaacctgctgagcgaaatgaaggcgcacgcgctggatatgaacgaggcgatccagcgtggtagcttcgttgaatttggccgtctggtgggtaaaacctgggagcaaaacaaagcgctggacagcggtaccaacccgccggcggtggaagcgatcattgatctgattaaggactacaccctgggctataaactgccgggtgcgggtggcggtggctacctgtatatggttgcgaaggacccgcaggcggcggtgcgtatccgtaaaattctgaccgagaacgcgccgaacccgcgtgcgcgtttcgttgagatgaccctgagcgataaaggtttccaagtgagccgcagctaa

SEQ ID NO: 6 - DNA, 564 bp, Artificial Sequence
  Description of Artificial Sequence Synthetic polynucleotide
atggccagcagcatgggtctggaaaccgttccagccggtaaagcgctgccggacgacatctacgtggttattgagatcccggcgaacagcgacccgatcaagtatgaagtggacaaggagagcggcgcgctgttcgtggatcgcttcatggccaccgcgatgttttatccggccaactacggctacgtgaacaataccctcagtctggatggcgatccagtggatgttctcgtgccaaccccatacccactgcagccgggcagcgttattcgctgccgtccggttggcgtgctgaaaatgaccgatgaagcgggcagcgatgcgaaagttgtggcggttccgcatagcaagctgaccaaggagtacgaccacatcaaagacgtgaatgatctgccggcgctgctgaaagcccagatccagcacttcttcgagagctacaaagcgctggaagccggtaaatgggtgaaagtggatggctgggaaggcgttgatgcggcccgccaagagattctggatagcttcgaacgcgcgaagaaactcgagcaccaccaccaccaccactga

SEQ ID NO: 7 - DNA, 741 bp, Artificial Sequence
  Description of Artificial Sequence Synthetic polynucleotide
atgggcagcagccatcatcatcatcaccacagcagcggtctggttccacgtggcagcatgaccgccatcgccccagttatcaccattgatggcccgagcggtgcgggtaaaggtacgctgtgcaaagcgatggcggaagcgctgcaatggcatctgctggatagcggcgccatctatcgcgttctcgcgctggcggcgctgcatcatcacgtggatgtggcgagcgaagacgcgctggttccgctggccagccatctggatgtgcgctttgtgagcaccaacggcaatctggaggttattctggagggcgaagatgttagcggcgaaatccgcacgcaagaagttgccaatgcggcgagtcaagttgccgcctttccacgcgttcgtgaagcgctgctgcgccgtcagcgtgcctttcgtgaactgccgggtctgatcgccgatggtcgtgatatgggcacggttgtgttcccggatgccccggtgaaaatctttctggacgccagcagtgaagaacgtgcgcaccgccgtatgctgcagctgcaagttaagggtttcagcgtgaacttcgagcgtctgctggcggaaatcaaagaacgcgatgaccgcgaccgcaatcgtgccgttgccccactggttccagcggccgatgccctcgttctggatagcacgacgctgagcatcgaacaagttattgagaaagcgctgcagtacgcccgccagaaactggcgctggcctaa

SEQ ID NO: 8 - DNA, 489 bp, Artificial Sequence
  Description of Artificial Sequence Synthetic polynucleotide
atgggcagcagccatcatcatcatcatcacagcagcggcctggtgccgcgcggcagcatggccatcgagcgcacctttagcatcatcaagccaaacgccgttgcgaagaacgtgatcggcaacatcttcgcccgtttcgaagccgccggtttcaaaatcgtgggcaccaagatgctgcatctgaccgttgaacaagcccgcggcttttacgcggaacacgatggcaaaccgttcttcgacggtctggtggaattcatgaccagcggcccgatcgtggttagcgtgctggaaggcgaaaatgccgttcagcgccatcgcgatctgctgggtgcgaccaatccagccaatgcgctggcgggtacgctgcgtgcggattacgccgacagcctcaccgaaaacggcacgcatggcagcgatagcgtggaaagcgccgcccgtgaaatcgcctatttcttcggcgagggtgaagtgtgcccacgtacgcgctaa

SEQ ID NO: 9 - DNA, 4743 bp, Artificial Sequence
  Description of Artificial Sequence Synthetic polynucleotide
atgggaagtgatgataatctggcgctgaaccaaacggttaccgcgagcagttacgaggttgccaccaccgcgccggaaaaggccgtggatggtgatctgggcacccgttggggtaccgcccagaataaagccgcgaatgagtggattgaagttggtctgggcggtacgaagacggtgaagcagatcaacatcgatttcgagcgcaaggatgccgaccagaacatcacgagcttcaaggtggagctcaaacaaggcgacacgtacacgaaggtttatcagaaagatacccgcgcgaagcagcaagaaatcattctgctggatcaagcccagcaagccagtgcggtgaaagtgacggttctgagcgcggatggtggtacgatgaactgggttaacgtgggcatcaacgagatcagcgtttacagcgcgccaaaagaaaccgtgctggacacggcggatacgaatcacatgctgggcgcgacgatgaccgcgagcagcaacgaaacggcgacgctgacgccagataaggcgatcgaccagaaccgcaccggccgcaataatcgctgggcgagtggctacgagacgccgagtaacatctggctgaaggccgaattcccgcgcctcaccgcggttaaggacatccgcatctacttcttcgagcgcgacgttaatccaaaaccgacgaacgtgcagagtttcgatctgagctacaccgatagcgaaggtacggaacacaccctcaagagcggttatgcgatgacggcgagcggtgcgggttatgttgccgatgtggtgattcaactcgaccaagccgtgaacgcccgtagtctcaagctgagcaacttcgcgatcaaaagcagcgaatataataatgttagcgttgcggagtgggaggcgtatagtaatgatcaagccgagccgggtgcgacgctggatagtgttgttagcgatctggagagcaaccacctcaccattgaaacggatacggacacgctggcgctgccaacggttccagacggctataccgtgaagttcaacggcgcggattacgagcaactgattgccgccgatggcaccgtgaaccatccgctggtggacaagaccgtgcaagttgcgtacgttgtgacggataccgccaccggtaataccaagaccaccagcgacatcccatacgtggttaagggcacgaatcagcagcaagagggtaacaacgccaaaccgaccatcatcccggaaattgcggaatggcatagcaccagcgcggccaagctggcggccagtgccgtgacgaaggtggtttacgacgacgacagcctcaaggcggtggtggatgagttcgtggccgactacaaggatttcacgggcatcaagctgacggccaagaaaggcgccgcggaagccggcgccttcaactttgtgaaaaccgacagcacggcggccattgcgcaactgggcgatgaaggctacacgatggacattcgcgccgatcgcgtggttgccaaaagcagcagcgtgacgggtaacatgtacgccatgcaaaccattctgcagatgaccaagcaagacgcgaatggtttcgttatcggcagcatgcgtgattacccacgcttcaccacgcgtggtctgctgctggacgtggcgcgcaaaccagtgagtctggaaatgatgcgtgagatcacccgcaccatgcgctactacaagatgaacgacttccaagcccatctgagtgacaattacatttttctggaaaactatggcaagggcgataacgaagacgaggcctttaaagcctacgatgcgtttcgtctggagagtagtctgacgaacgacaagggtgaaagcccgaccgcggaagactatagcatcagtaagaaaacgttcaagcagtttatccaagatgaacgcgcgctgggcatgaacgtggttccagagattgacgtgccggcgcatgccaacagctttacgaagatctggccggagctgatggtgaaaggtcgcgtgagtccgatcaacagtaaccgtccactgatcgatcacctcgatgttagcaagccggagacgatcgcgaaaatcaaggagatcttcgacgactacaccaagggcgacgatccgaccttcgacagcgacacgacggttcatattggcgcggaagagtttctgtataactataccgcgtatcgtaagttcatcaatgaaatcgttccatatatcaaagatacgaacaccgtgcgtatgtggggtggtctgacgtggatcaatgaccacaagaccgagattacgaaggacgcgatcgagaacgtggaaatgaatctgtggagtaaggactgggccgacggtctccagatgtacaatatgggttataagctgattaatacgatcgacgactacggctacatggtgccaaatggcagttacggccgcgccaacgcctatggtgatctgctcaacatcagccgtgtgttcgacagctttgagccaaacaaggtgcgcagtagcggcggctatcaagcggtgccaagtggcgatgatcagatgctgggtgcggcctttgccatctggagcgataacatcgataaaagcgccagcggtctgacggaaagtgatctgtactggcgcttcttcgacgccatgccattttacgcggagaaaacgtgggcggcgacgggcaaagagaaaggtaccgcggcgaagctgacggcgctggcggccaaacaaggcacgggcccacgcaccaatccgtactaccaagccaccagcaagaacagtgtttacgagagctatgactttaacgatggtctggccgatgccagtggtaacggtcgcgatctcacgattggcgatggcagtaaagcggccgtgaaagatcagagcctcaaactggccggcggtagcagctatgccacgagcaagctcgataaactcggcaacggcaacgagctcacctttgatgtgacgctgcaacaagccgcgaaaccgggcgatatcctcttcgaagccgacgcgccgtacggtacccacgatatccgtgtgatggagaacggcaaactgggcttcacgcgcgaactctacaattactactttgattacgagctgccggtgggcaaaacggttacggttaccatcaaggttgaccagcagaccaccaaactgtatgtggacggcgaattcgtgagtgacgcgaccggcaagtacatcgacaagggcatcgagaagaaaacgggtattaccgccgccacctttgcgctcccgctgcagcgtatcggcagtaagaccagcgccatcaatggcgtgattgacaatgtgattgtgaagaagagcgaggccgaaaccgaccagtacaacaagagttgttggaccggcacgaccaacagcgagacgcaatacaacgataccgagggtctgctgcgctacgccttcgacaacaatccgagtaccatctggcacagcaattggaaaggtgcgaccgataagctgacgggcagtaacagcttttatgcggaaattgacatgtgtcagaaatacacgattaaccagttcagctttacgccgcgtaccagccaagatagcggccaagtgacgaaagccgatctgtatgtgaaggccaatgcgaacgacgaatggaaacaagttgccaccgatcaagtgtttgaggcgagtcgcgccaaaaagacgtttatgttcgacgagcaagaagtgcgttacgtgaaattcgtggccaagagcagtaacgatggttgggttgccgtgagtgagttcggcgtggccaataagccgagcagtaccgttcgcgtttttgttgccgcggatccagcggagggtggtaccgttagtgttgccgccgaaggcgaaacgggtaccgatacggcggtggacgttgccagcggtgccagtgttaccgcgaaagcggtggcggcggacggctatcgcttcagtggttggtttacgaccgcgagtgaaaccgccgtgagcaccgacgcgacctataccttcgcggccgatggtaataccgcgctgaccgcgaaattcaccaaagatagcaccccagatccgggcccgaagccaaccatcagtagtatcgcggtgacgaagccaaccgtgaccgattacaaagtgggcgataccttcgacgcgaccggtctcgccgtgaccgccacgatgagcgatggcagcacgaagacgctgaccgccggtgagtatacgctcagcgccacccaagatggcgcggccgttgcgctcgacaaagcctttgccaaagccggtaaggttacggtgacggtgacggccaatggtaaaacggccaccttcgacgtgaccgttacggcgaaagacccagacccggaaccggcgacgctgaaaagcatcaaggtgaccagcaaaccggataagaccacgtacacggttgacgaaaccttcgccaaaaccggtctggccgttaccggtacgtggagtgatggtaagaccgcgctgctcaaggatggtgaatacaagctcagcgcggttgacgccgacggcaagacggttgatctcacgaagccgttcacggccgcgggtgatgtgacggtgaccgttaccagcggtaagctgaccgacagctttaccatcaccgtgaaagccaaaaccgtgaccccggccccgggtgataataagccgggtgagaacaagccgggtgccgacaagccaaagccgaataccccagacgaagttgccaaaggtggtggcggtagccaccatcaccatcaccactaa

SEQ ID NO: 10 - protein, 413 aa, Artificial Sequence
  Description of Artificial Sequence Synthetic polypeptide
MPSSHHHHHHDYDIPTTENLYFQGSMKTITLYLDPASLPALNQLMDFTQNNEDKTHPRIFGLSRFKIPDNIITQYQNIHFVELKDNRPTEALFTILDQYPGNIELNIHLNIAHSVQLIRPILAYRFKHLDRVSIQQLNLYDDGSMEYVDLEKEENKDISAEIKQAEKQLSHYLLTGKIKFDNPTIARYVWQSAFPVKYHFLSTDYFEKAEFLQPLKEYLAENYQKMDWTAYQQLTPEQQAFYLTLVGFNDEVKQSLEVQQAKFIFTGTTTWEGNTDVREYYAQQQLNLLNHFTQAEGDLFIGDHYKIYFKGHPRGGEINDYILNNAKNITNIPANISFEVLMMTGLLPDKVGGVASSLYFSLPKEKISHIIFTSNKQVKSKEDALNNPYVKVMRRLGIIDESQVIFWDSLKQL

SEQ ID NO: 11 - protein, 253 aa, Artificial Sequence
  Description of Artificial Sequence Synthetic polypeptide
MPSSHHHHHHDYDIPTTENLYFQGSMEKQNIAVILARQNSKGLPLKNLRKMNGISLLGHTINAAISSKCFDRIIVSTDGGLIAEEAKNFGVEVVLRPAELASDTASSISGVIHALETIGSNSGTVTLLQPTSPLRTGAHIREAFSLFDEKIKGSVVSACPMEHHPLKTLLQINNGEYAPMRHLSDLEQPRQQLPQAFRPNGAIYINDTASLIANNCFFIAPTKLYIMSHQDSIDIDTELDLQQAENILNHKES

SEQ ID NO: 12 - protein, 542 aa, Artificial Sequence
  Description of Artificial Sequence Synthetic polypeptide
MASSHHHHHHGSSVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKLGSASMDFKIVQVHGGLGNQMFQYAFAKSLQTHLNIPVLLDTTWFDYGNRELGLHLFPIDLQCASAQQIAAAHMQNLPRLVRGALRRMGLGRVSKEIVFEYMPELFEPSRIAYFHGYFQDPRYFEDISPLIKQTFTLPHPTEHAEQYSRKLSQILAAKNSVFVHIRRGDYMRLGWQLDISYQLRAIAYMAKRVQNLELFLFCEDLEFVQNLDLGYPFVDMTTRDGAAHWDMMLMQSCKHGIITNSTYSWWAAYLIKNPEKIIIGPSHWIYGNENILCKDWVKIESQFETKS

SEQ ID NO: 13 - protein, 554 aa, Artificial Sequence
  Description of Artificial Sequence Synthetic polypeptide
MKSSHHHHHHGSSVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYKLGSDLVPRGSMIIVHLCGGLGNQMFQYAAGLAAAHRIGSEVKFDTHWFDATCLHQGLELRRVFGLELPEPSSKDLRKVLGACVHPAVRRLLAGHFLHGLRPKSLVIQPHFHYWTGFEHLPDNVYLEGYWQSERYFSNIADIIRQQFRFVEPLDPHNAALMDEMQSGVSVSLHIRRGDYFNNPQMRRVHGVDLSEYYPAAVATMIEKTNAERFYVFSDDPQWVLEHLKLPVSYTVVDHNRGAASYRDMQLMSACRHHIIANSTFSWWGAWLNPRPDKVVIAPRHWFNVDVFDTRDLYCPGWIVL

SEQ ID NO: 14 - protein, 974 aa, Artificial Sequence
  Description of Artificial Sequence Synthetic polypeptide
MPSSHHHHHHDYDIPTTENLYFQGSMQKLLSLPSNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILMKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAVSVFPMVNSVEELGLVLRWMLSEPELEEGKNIWLRSEHFSADEISAGANLKRLYAQREEFRKGNWKALAVNHEKSVFYQLDLADAAEDFVRLGLDMPELLPEDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 15 - protein, 187 aa, Artificial Sequence
  Description of Artificial Sequence Synthetic polypeptide
MASSMGLETVPAGKALPDDIYVVIEIPANSDPIKYEVDKESGALFVDRFMATAMFYPANYGYVNNTLSLDGDPVDVLVPTPYPLQPGSVIRCRPVGVLKMTDEAGSDAKVVAVPHSKLTKEYDHIKDVNDLPALLKAQIQHFFESYKALEAGKWVKVDGWEGVDAARQEILDSFERAKKLEHHHHHH

SEQ ID NO: 16 - protein, 246 aa, Artificial Sequence
  Description of Artificial Sequence Synthetic polypeptide
MGSSHHHHHHSSGLVPRGSMTAIAPVITIDGPSGAGKGTLCKAMAEALQWHLLDSGAIYRVLALAALHHHVDVASEDALVPLASHLDVRFVSTNGNLEVILEGEDVSGEIRTQEVANAASQVAAFPRVREALLRRQRAFRELPGLIADGRDMGTVVFPDAPVKIFLDASSEERAHRRMLQLQVKGFSVNFERLLAEIKERDDRDRNRAVAPLVPAADALVLDSTTLSIEQVIEKALQYARQKLALA

SEQ ID NO: 17 - protein, 162 aa, Artificial Sequence
  Description of Artificial Sequence Synthetic polypeptide
MGSSHHHHHHSSGLVPRGSMAIERTFSIIKPNAVAKNVIGNIFARFEAAGFKIVGTKMLHLTVEQARGFYAEHDGKPFFDGLVEFMTSGPIVVSVLEGENAVQRHRDLLGATNPANALAGTLRADYADSLTENGTHGSDSVESAAREIAYFFGEGEVCPRTR

SEQ ID NO: 18 - protein, 1580 aa, Artificial Sequence
  Description of Artificial Sequence Synthetic polypeptide
MGSDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGAEEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAKGGGGSHHHHHH

(19) United States
(12) Patent Application Publication
(10) Pub. No.: US 2024/0132927 A1
(43) Pub. Date: Apr. 25, 2024

(54) MODULAR GLYCAN PRODUCTION WITH IMMOBILIZED BIONANOCATALYSTS

(71) Applicant: ZYMtronix Catalytic Systems, Inc., Ithaca, NY (US)

(72) Inventors: Stephane Cedric Corgie, Ithaca, NY (US); Alexander Chris Hoepker, Ithaca, NY (US)

(73) Assignee: ZYMtronix Catalytic Systems, Inc., Ithaca, NY (US)

(21) Appl. No.: 18/038,695

(22) PCT Filed: Dec. 1, 2021
(86) PCT No.: PCT/US2021/061493

§ 371 Date: May 24, 2023

Related U.S. Application Data
(60) Provisional application No. 63/120,669, filed on Dec. 2, 2020.

(51) Int. Cl. C12P 19/04; C12N 9/10; C12N 9/12; C12N 9/16; C12N 11/14

(52) U.S. Cl. CPC C12P 19/04; C12N 9/1051; C12N 9/1081; C12N 9/1229; C12N 9/1241; C12N 9/16; C12N 11/14; C12Y 204/01069; C12Y 204/99006; C12Y 207/01052; C12Y 207/04006; C12Y 207/04014; C12Y 207/07043; C12Y 305/01052; C12Y 306/01001

(57) ABSTRACT

The invention provides modular cell-free de-novo synthesis of glycans with immobilized bionanocatalysts. The invention provides materials, and in particular, magnetic materials, for producing glycans of defined length and sequences using one or more enzymes that are immobilized within bionanocatalysts (BNCs) which in turn are embedded within scaffolds to control the synthesis in batch or continuous processes manufacturing. In some embodiments, the scaffolds are high magnetism and high porosity composite blends of thermoplastics or thermosets comprising magnetic particles that form powders. In some embodiments, Selective Laser Sintering (SLS) is used to design and produce objects via 3D printing by sintering composite magnetic powders. The modular flow cells may be mixed and matched for a highly customizable and highly efficient cell-free manufacturing process. In some embodiments the elementary and system modules provided by the invention are employed. In preferred embodiments, human milk oligosaccharides (HMOs) are produced.

### CROSS-REFERENCE TO RELATED APPLICATIONS

This application claims priority under 35 U.S.C. § 119(e) to U.S. Provisional Application No. 63/120,669, filed Dec. 2, 2020, and is incorporated herein by reference in their entirety.

### FIELD OF THE INVENTION

The invention provides modular cell-free de-novo synthesis of glycans with immobilized bionanocatalysts. The invention provides materials, and in particular, magnetic materials, for the modular production of glycans using one or more enzymes that are immobilized within bionanocatalysts (BNCs) which in turn are templated onto magnetic scaffolds. The templated BNCs may be inside of modular flow cells for flow manufacturing or may be used in batch processes. Accordingly, the invention provides cell-free productions of defined glycans with combinatorial bionanocatalysts (BNCs) organized in sequential modules. The modular flow cells may be mixed and matched for a highly customizable and highly efficient manufacturing process. In preferred embodiments, human milk oligosaccharides (HMOs) are produced.

### BACKGROUND OF THE INVENTION

Biocatalysis, as a green technology, has become increasingly popular in chemical manufacturing over traditional expensive and inefficient processes. Its applications include the production of food ingredients, flavors, fragrances, commodity and fine chemicals, and active pharmaceuticals. When producing chemicals at industrial scale, however, enzymes can suffer drastic losses in activity and loading causing a significant drop in performance.

Magnetic enzyme immobilization involves the entrapment of enzymes in mesoporous magnetic clusters that self-assemble around the enzymes (level 1). The immobilization efficiency depends on a number of factors that include the initial concentrations of enzymes and nanoparticles, the nature of the enzyme surface, the electrostatic potential of the enzyme, the nature of the nanoparticle surface, and the time of contact. Enzymes used for industrial purposes in biocatalytic processes should be highly efficient, stable before and during the process, reusable over several biocatalytic cycles, and economical.

Mesoporous aggregates of magnetic nanoparticles may be incorporated into continuous or particulate macroporous scaffolds (level 2). The scaffolds may or may not be magnetic. Such scaffolds are discussed in WO2014/055853, WO2017/180383, and Corgie et al., Chem. Today 34(5):15-20 (2016), incorporated by reference herein in their entirety. Highly magnetic scaffolds are designed to immobilize, stabilize, and optimize any enzyme. This includes full enzyme systems, at high loading and full activity, and for the production of, e.g., small molecules.

Selective laser sintering (SLS) is an additive manufacturing (AM) technique that uses a laser as the power source to sinter powdered materials such as plastic, metal, ceramic, glass powders, nylon or polyamide. A laser automatically aimed at points in space, defined by a 3D model (e.g. an Additive Manufacturing File, AMF, or a CAD file), binds the material together to create a solid structure. After each cross-section is scanned, the powder bed is lowered by a one-layer thickness, a new layer of material is applied on top, and the process is repeated until the part is completed. There are many different technologies, such as stereolithography (SLA) or fused deposit modeling (FDM).

SLS is similar to direct metal laser sintering (DMLS) but differs in technical details. DSLM uses a comparable concept, but in DSLM the material is fully melted rather than sintered. This allows one to manufacture materials with different properties (e.g. crystal structure and porosity). SLS is a relatively new technology that may be expanded into commercial-scale manufacturing processes.

Continuous flow processing begins with two or more streams of different reactants pumped at specific flow rates into a single cell. A reaction takes place, and the stream containing the resultant compound is collected at the outlet. The solution may also be directed to subsequent flow reactor loops to generate the final product. Continuous flow processing provides better control and reproducibility of reactions. It is a modular, customizable approach. The high modularity allows one to configure the cells to meet the requirements of specific reactions.

Glycans are complex carbohydrate structures that are the predominant molecules on the cell surface and serve as the first point of contact between cells, the extracellular matrix and pathogens. There is great interest in improving the accessibility and affordability of these molecules for research, preclinical and commercial applications.

A recent study by the CDC found that roughly 1-in-5 mothers of newborns never initiate breastfeeding and the majority (>70%) do not meet the 6-month target, instead relying extensively or exclusively on commercially available infant formula. Non-compliance with breastfeeding recommendations is estimated to add an additional $2.5 billion to pediatric healthcare costs (direct) in the US with the total burden of morbidity and mortality totaling $13.8 billion. (Vera M, LC NM. Implementation of Mother-friendly Workplace Policies in Hong Kong, HKJGOM 2015; 15(1) 11.

Human milk oligosaccharides (HMOs) are the third largest component of breast milk. They are particularly commercially relevant. For instance, they serve as metabolic substrates for specialized beneficial bacteria, thereby shaping the intestinal microbiome. More complex and branched HMOs (>4 DP (i.e., degree of polymerization), however, appear mostly prophylactic and serve as soluble decoys for viral, bacterial, or protozoan parasite adhesins, thereby preventing attachment to the infant, or adults, mucosal surface.

Moreover, HMOs can also modulate epithelial and immune cell responses and reduce excessive mucosal leukocyte infiltration and activation. These properties have been associated with a lower risk for developing necrotizing enterocolitis and other infections and autoimmune inflammations.

While some simple probiotic HMOs can be effectively produced via fermentation for infant formula (2′FL, 3DP), complex and branched HMOs are elusive. Microbial production is limited to simple unbranched and short-length sugars and bacteria can secrete toxins that must be filtered out. Yeast fermentation does not require the removal of toxins, and the overall process involves fewer steps, reducing production costs and resulting in a more easily scaled product. However, engineering organisms is intensive and does not guarantee the ability to scale-up.

More complex, branched polysaccharides have been marginally produced at high cost via chemocatalysis. In order to make HMOs, sugars need to be first activated to be able to be sequentially transferred to the growing structures. For example, activated fucose (L-Fucose-1-GDP) and activated sialic acid (Sialic acid-1-CMP) cost $35,000 and $8,000 per gram respectively, while L-fucose and L-sialic acid only cost $10 and $5.5 per gram, putting the cost of any complex glycans in hundreds of thousand dollars per gram—the current cost of defined DP5 glycans, for example, ranges $300,000 and $3,800,000 per gram. https://academic.oup.com/glycob/article/13/7/41R/612936

Cell free systems have been of interest in biomanufacturing, but there are challenges (M.P. Cordoso Marque et al., Adv. Biochem. Eng. Biotechnol., https://doi.org/10.1007/10_2020_160 and C. You, Adv. Biochem. Engl, DOI: 10.1007/10_2012_159 (2012)).

Thus, the art seeks an economical and efficient way to produce glycans, including HMOs, and including glycans larger than 5 sugar units. This could overcome major hurdles in advancing these glycans and HMOs for probiotic, prophylactic and therapeutic purposes, including infant nutrition and disease prevention. Importantly, the invention allows to enhance production efficiency of complex carbohydrates while lowering costs, hence improving the accessibility and affordability of these molecules.

### SUMMARY OF THE INVENTION

The invention provides modular cell-free de-novo synthesis of glycans with immobilized bionanocatalysts. The invention significantly improves efficiency and reduces cost for the production of glycans, including but not limited to, oligosaccharides, including but not limited to, large and complex HMOs of SDP or more, and large and complex HMOs (>5 DP).

The invention provides materials, and in particular, magnetic materials, for producing glycans oligosaccharides, including, but not limited to, five sugar units or more using one or more enzymes that are immobilized within bionanocatalysts (BNCs) which in turn are embedded within scaffolds. Bionanocatalysts (BNCs) according to this invention comprise an enzyme self-assembled with magnetic nanoparticles (MNPs). The BNCs self-assemble with the scaffolds.

In certain embodiments, the scaffolded BNCs are inside of modular flow cells for flow manufacturing. In certain embodiments, the invention provides continuous flow processing where each step of synthesis is conducted in modules. In production mode, these modules contain full systems of enzymes—sugar activation and sugar transfer—for specifically building glycans. In some embodiments, the glycans are oligosaccharides.

In some embodiments, the scaffolds comprise magnetic metal oxides. In some embodiments, the scaffolds are high magnetism and high porosity composite blends of thermoplastics comprising magnetic particles that form powders. They may be single-layered or multiple-layered materials that hold the BNCs. Such designed objects may be produced using 3D printing by sintering composite magnetic powders. In some embodiments, Selective Laser Sintering (SLS) is used. The modular flow cells may be mixed and matched for a highly customizable and highly efficient manufacturing process. In preferred embodiments, human milk oligosaccharides (HMOs) are produced.

Thus, the invention provides cell-free productions of defined glycans with combinatorial bionanocatalysts (BNCs) organized in sequential modules.

### BRIEF DESCRIPTION OF THE DRAWINGS

FIG. 1 is a description of elementary modules. The legend describes the identity of sugar symbols and sugar shorthand pertaining to all figures and the body of the text, including description and claims.

FIG. 1A depicts module EM-1-1A (EM-1-1-8-1) comprising BNCs of enzyme BfFKP (bifunctional L-fucokinase/GDP-L-Fuc pyrophosphorylase; from Bacteroides fragilis). See Example 4. BfFKP converts L-fucose, ATP and GTP to L-fucose-GDP, ADP and inorganic diphosphate PPi. The module is termed an activation elementary module that activates L-fucose for functionalization (fucosylation) of a sugar moiety.

FIG. 1B depicts module EM-1-3A (EM-1-3-1-2) comprising BNCs of enzyme StGalE (UDP-4′-gal-epimerase=StGalE; StGalE from Streptococcus thermophilus; enzyme is expressed) that converts Glc-UDP to Gal-UDP and then transfers Gal to GlcNAc while expelling UDP. The module is termed an activation elementary module EM-1-3A that produce UDP-Gal for the elongation of HMO core structure.

FIG. 1C depicts module EM-1-4A (EM-1-4-1-1) comprising BNCs of enzyme NmCSS (CMP—sialic acid synthetase from Neisseria meningitidis; See Example 4.) that converts Neu5Ac and CTP to Neu5Ac-CMP and inorganic diphosphate PPi. The module is termed an activation elementary module that activate Neu5Ac for functionalization (sialylation) of a sugar moiety.

FIG. 1D depicts module EM-2-2A (EM-2-2-3-1-1) comprising BNCs of enzyme BbhI (β-N-acetylglucosaminidase from Bifidobacterium bifidum; see Example 4) that transfers GlcNAc from chemically synthesized GlcNAc-oxazoline to Gal with an β-1,3 linkage. The module is termed an extension elementary module that elongates the HMO core structure.

FIG. 1E depicts module EM-2-3A (EM-2-3-1-1-1) comprising BNCs of enzyme Te2FT (α1,2-fucosyltransferase from Thermosynechococcus elongatus; see Example 4) that transfers L-fucose-GDP to a Gal acceptor to form an α-1,2-fucosylated product while expelling GDP. The module is termed an extension elementary module that functionalizes HMO core structures with L-fucose.

FIG. 1F depicts module EM-2-3B (EM-2-3-1-1-2) comprising BNCs of enzyme WbgL (α1,2-fucosyltransferase from Escherichia coli; see Example 4) that transfers L-fucose-GDP to a Gal acceptor to form an α-1,2-fucosylated product while expelling GDP. The module is termed an extension elementary module that functionalizes HMO core structures with L-fucose.

FIG. 1G depicts module EM-2-3C (EM-2-3-1-2-2) comprising BNCs of enzyme Bf13FT (α1,3,4-fucosyltransferase from Bacteroides fragilis) that transfers L-fucose-GDP and a GlcNAc or Glc acceptor to form an α-1,3-fucosylated or α-1,4-fucosylated product while expelling GDP. The module is termed an extension elementary module that functionalizes HMO core structures with L-fucose.

FIG. 1H depicts module EM-2-3D (EM-2-3-2-1-1) comprising BNCs of enzyme PmST1 (α-2,3-sialyltransferase from Pasteurella multocida wild or its mutants; see Example 4) that transfers Neu5Ac from Neu5Ac-CMP produced from module EM1.4A to Gal with an α-2,3 linkage while expelling CMP. The module is termed an extension elementary module that functionalizes HMO core structures with Neu5Ac.

FIG. 1I depicts Module EM2-3-E (EM-2-3-2-2-2) comprising BNCs of enzyme ST6GAL1 (α-2,6-sialyltransferase from Homo sapiens) that transfers Neu5Ac from Neu5Ac-CMP to Gal with an α-2,6 linkage while expelling CMP. The module is termed an extension elementary module that functionalizes HMO core structures with Neu5Ac.

FIG. 1J depicts module EM-2-3F (EM-2-3-2-2-8) comprising BNCs of enzyme STGALNAC5 (α-2,6-sialyltransferase from Homo sapiens; supplied by Glycoexpression Technologies) that transfers Neu5Ac from Neu5Ac-CMP to GlcNAc with an α-2,6 linkage while expelling CMP. The module is termed an extension elementary module that functionalizes HMO core structures with Neu5Ac.

FIG. 1K depicts module EM-2-3G (EM-2-3-5-1-1) comprising BNCs of Cvβ3GalT (β-3-galactosyltransferase from Chromobacterium violaceum; see Example 4) that transfers Gal to a GlcNAc residue while expelling UDP. The module is termed an extension elementary module that elongates HMO core structures to make type I structures.

FIG. 1L depicts module EM-2-3H (EM-2-3-5-2-1) comprising BNCs of NmLgtB (β-4-galactosyltransferase from Neisseria meningitidis; see Example 4) that transfers Gal to GlcNAc while expelling UDP. The module is termed an extension elementary module that elongates the HMO core structures to make type II structures.

FIG. 1M depicts module EM-3-1A (EM-3-1-2-2) comprising BNCs of enzyme NDPK (Nucleoside 5′-Diphosphate Kinase from E. coli; see Example 4) which recycles the GDP produced by fucosyltransferases to GTP with equimolar amounts of ATP that is converted to ADP. The module is termed a regeneration elementary module that regenerates GTP from GDP.

FIG. 1N depicts Module EM-3-1C (EM-3-1-3-2) comprising BNCs of enzymes CMPK (Cytidine 5′-Monophosphate Kinase from E. coli; see Example 4) and NDPK (Nucleoside 5′-Diphosphate Kinase from E. coli; expressed) which convert CMP to CTP with the consumption of two molar equivalents of ATP. The module is termed a regeneration elementary module.

FIG. 1O depicts module EM-3-2A (EM-3-2-1-1-1) comprising BNCs of enzyme SuS (AtSuS1, sucrose synthase from Arabidopsis thaliana) which converts UDP and sucrose to Glc-UDP and fructose. The module is termed a regeneration elementary module that regenerates Glc-UDP from UDP.

FIG. 1P depicts module EM-5-1A (EM-5-1-1) comprising BNCs of enzyme PmPpa (Inorganic pyrophosphorylase, from Pasteurella multocida; see Example 4) which converts pyrophosphate into two equivalents of inorganic phosphate. The module is termed a support elementary module that shifts the equilibria of sugar activation steps.

FIG. 1Q depicts module EM-6-3A (EM-6-3-1) comprising BNCs of enzyme exo-α-sialidase (exo-α-sialidase, from Salmonella typhimurium; enzyme obtained) which removes a terminal Neu5Ac residue from HMO structures. The module is termed a sugar-removal elementary module that removes a Neu5Ac residue.

FIG. 2 depicts elementary modules and their use in the synthesis of LNTII, LNT, LNnT, LNFPI, LNFPII, LNFPIII, LSTa, LSTb, LSTc, LSTd, DSLNT, and 2′-FL. The elementary modules comprise BNCs that are combined into sets of system modules (see FIG. 1).

FIG. 2, Step A depicts the synthesis of LNTII using SM6 (see FIG. 1 and Example 2c). Lactose is converted to LNTII using SM6 and the enzyme of EM-2-2A.

FIG. 2, Step B depicts the synthesis of LNnT using SM3 see (FIG. 1 and Example 2c).

FIG. 2, Step C depicts the synthesis of LNT using SM3 (see FIG. 1 and Example 2c).

FIG. 2, Step D depicts the synthesis of 2′-FL using SM1 (see FIG. 1 and Example 2c). Lactose is converted to 2-FL using SM1 and the enzymes of EM-1-1A, EM-2-3B, EM-3-1A, and EM-5-1A.

FIG. 2, Step E depicts the synthesis of LNFPII using SM1 (see FIG. 1 and Example 2c).

FIG. 2, Step F depicts the synthesis of LNFPI using SM1 (see FIG. 1 and Example 2c).

FIG. 2, Step G depicts the synthesis of LSTc using SM4 (see FIG. 1 and Example 2c).

FIG. 2, Step H depicts the synthesis of LNFPIII using SM1 (see FIG. 1 and Example 2c).

FIG. 2, Step I depicts the synthesis of LSTd using SM4 (see FIG. 1 and Example 2c).

FIG. 2, Step J depicts the synthesis of LSTa using SM4 (see FIG. 1 and Example 2c).

FIG. 2, Step K depicts the synthesis of DSLNT using SM4 (see FIG. 1 and Example 2c)

FIG. 2 Step L depicts the synthesis of LSTb using SM11 (see FIG. 1 and Example 2c).

FIG. 3 depicts the synthesis of DSDFLNnH, via Step A, Step B, and Step C using SM3, SM4, and SM1 respectively (see FIG. 1 and Example 2d-C).

FIG. 4A depicts batch reaction synthesis of 3′SL with scaffolded BNCs and a HPLC analysis of the reaction (see Example 1b). The reaction afforded greater than 75% conversion of 3′-SL from Neu5Ac by HPLC analysis (see Example 1b). That yield corresponded to −90% of the yield compared to the free enzyme reaction.

FIG. 4B depicts in-flow synthesis of 3′SL with immobilized enzymes, presents a % conversion to 3′-SL as determined by HPLC, and presents a representative HPLC chromatogram (see Example 1c).

FIG. 4C depicts the enzymatic reaction scheme and % conversion of lactose to 3′-SL over time using scaffolded BNCs in a batch reaction. The % conversions were determined by HPLC and are shown for two enzyme loadings (1× and 5×). See Example 3b for details.

FIG. 4D depicts the enzymatic reaction scheme and % conversion of lactose to 3′-SL over time using scaffolded BNCs in a flow reaction. The % conversions were determined by HPLC and a representative HPLC chromatogram is depicted with the signal measured amperometrically (picoAmps, pA) by charged aerosol detection (CAD). See Example 3c for details.

FIG. 5 depicts the enzymatic reaction scheme and % conversion of lactose to LNTII over time using scaffolded BNCs in a flow reactor. The % conversions were determined by HPLC and a representative HPLC chromatogram is depicted with the signal measured amperometrically (picoAmps, pA) by charged aerosol detection (CAD). See Example 3c-A for details.

FIG. 6 depicts the enzymatic reaction scheme and % conversion of lactose to 2′-FL over time using NiNTA immobilized enzymes in a flow reactor. The % conversions were determined by HPLC and a representative HPLC chromatogram is depicted with the signal measured amperometrically (picoAmps, pA) by charged aerosol detection (CAD). See Example 3c-B for details.

FIG. 7 depicts the enzymatic reaction scheme and % conversion of LNT to LNFPI over time using scaffolded BNCs in a batch reaction. The % conversions were determined by HPLC and a representative HPLC chromatogram is depicted with the signal measured amperometrically (picoAmps, pA) by charged aerosol detection (CAD). The % conversions are shown for two enzyme loadings (1× and 5×). See Example 3c-C for details.

FIG. 8 depicts the enzymatic reaction scheme and % conversion of LNT to LSTa over time using scaffolded BNCs in a batch reaction. The % conversions were determined by HPLC and a representative HPLC chromatogram is depicted with the signal measured amperometrically (picoAmps, pA) by charged aerosol detection (CAD). The % conversions are shown for two enzyme loadings (1× and 5×). See Example 3c-D for details.

FIG. 9 depicts the enzyme immobilization scheme as employed for Examples 3b, 3c-A, 3c-B, 3c-C and 3c-D. In a first step, strontium ferrite (SFE) scaffold material is coated with magnetic nanoparticles (MNPs) by lower the pH from 10.0 to 7.5. In a second step, the enzymes are added to form the scaffolded BNCs.

FIG. 10 depicts the enzymatic reaction scheme for creating LNTII. In the first step, GlcNAc-oxa is produced by pumping GlcNAc, Et₃N and DMC into a jacketed CSTR cooled to 0° C. In the second step, the GlcNAc-oxa is pumped into an intermediate jacketed CSTR cooled to 0° C. along with buffered lactose and acid to maintain pH. This mixture is then pumped into a packed bed reactor containing the enzyme Bbh1 immobilized on the scaffold to produce LNTII. pHC=pH controller; DMC=2-Chloro-1,3-dimethylimidazolinium chloride; GlcNAc=N-acetylglucosamine; CSTR=continuous stirred-tank reactor; M=mixer; T=temperature controller; pH=pH probe.

### DETAILED DESCRIPTION OF THE INVENTION

The invention provides cell-free de-novo synthesis of glycans with immobilized bionanocatalysts, including cell-free productions of defined glycans with combinatorial bionanocatalysts (BNCs). The invention involves permanent molecular entrapment of enzymes within self-assembling nanoparticle (NP) clusters. The self-assembly is purely driven by the materials' electrostatic and magnetic interactions. Ionic strength, buffer pH, and NP concentration are the main parameters impacting the immobilization yield and optimized enzyme activity.

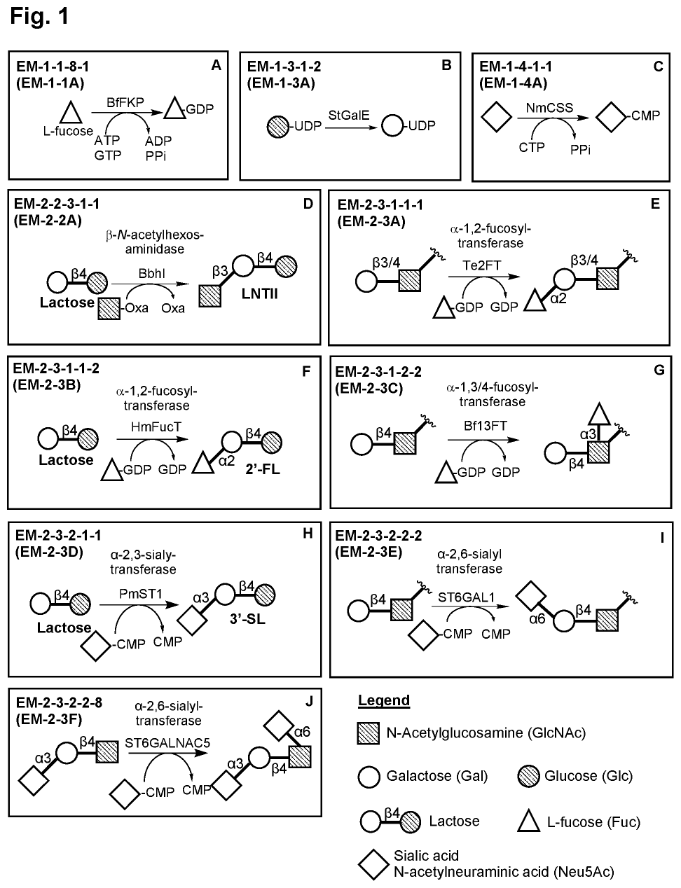

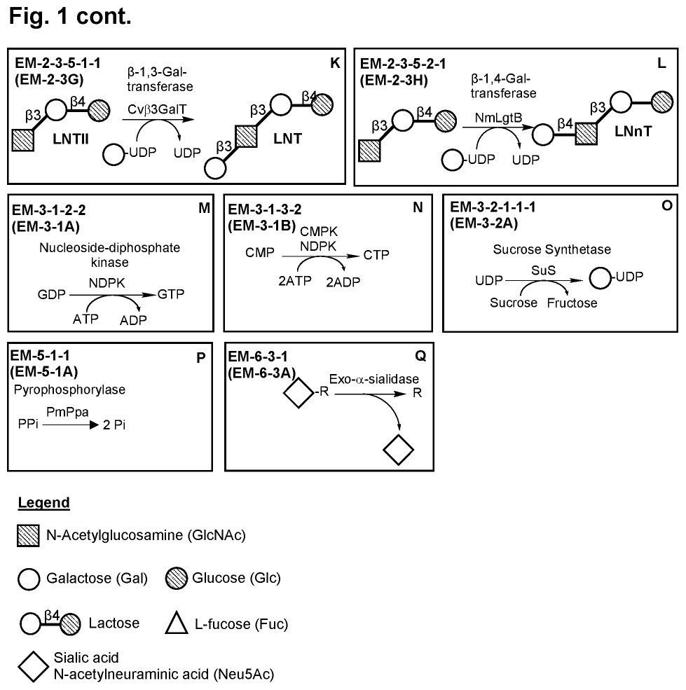

Bionanocatalysts (BNCs) according to this invention comprise an enzyme self-assembled with magnetic nanoparticles (MNPs). Self-assembled mesoporous aggregate of magnetic nanoparticles comprise a glycan synthesis enzyme, wherein the mesoporous aggregate is immobilized on a magnetic macroporous scaffold. In certain embodiments, the BNCs and microporous materials are as defined in herein, including Examples 1-5, Example 2, FIGS. 1-10, or any of the claims. Accordingly, this invention provides a composition comprising i. a glycan synthesis enzyme self-assembled in magnetic nanoparticles, and ii. a magnetic scaffold. A glycan enzyme is immobilized on nanoparticles where the nanoparticles coat a scaffold, and the enzyme is immobilized in or on the mesoporous structure formed by the nanoparticles. In one embodiment, the nanoparticles comprise magnetite (Fe₃O₄) or maghemite (Fe₂O₃). In another embodiment, the nanoparticles comprise a product synthesized from FeCl₂ and FeCl₃, particularly synthesized via continuous coprecipitation of FeCl₂*4H₂O and FeCl₃. FeCl₂*4H₂O (Iron (II) chloride tetrahydrate and FeCl₃*6H₂O (Iron (III) chloride hexahydrate. Accordingly, in one embodiment, the nanoparticles comprise magnetite (Fe₃O₄).

In one embodiment, the invention provides a module comprising a magnetic macroporous matrix material comprising self-assembled mesoporous aggregates of magnetic nanoparticles magnetically entrapping an immobilized glycan synthesis enzyme. In certain embodiments, the module is a system enzyme module. A system module may be combined with other system modules to synthesize glycans. In certain embodiments of the invention, the module consists essentially of or consists of metallic materials.

It should be understood that ‘comprise’ is, where context permits, to be interpreted non-exhaustively. In certain aspects of the invention, the embodiments, including but not limited to a scaffold or a scaffolded BNC, optionally do not include a polymer. Certain embodiments, including but not limited to a scaffold or scaffolded BNC, do not include a polymer. Certain embodiments including, but not limited to a scaffold or a scaffolded BNC, do not include polyvinyl alcohol or a thermoplastic polymer. Accordingly, in a module comprising a magnetic macroporous matrix, the material consists essentially of, or consists of, metallic materials and does not comprise a polymer.

A glycan synthesis enzyme is any enzyme that can be used in the synthesis of a glycan. Steps in glycan synthesis may include activating a sugar, transferring a sugar unit thereby extending a sugar, cofactor recycling, and equilibrium shifting. Glycan synthesis enzymes include, but are not limited to, a sugar activation enzyme, a sugar extension enzyme, a reagent regeneration enzyme, a sugar functionalization enzyme, a sugar support enzyme, a sugar removal enzyme. Accordingly, in one embodiment, the module comprises a self-assembled mesoporous aggregates comprising a single glycan synthesis enzyme or more than one glycan synthesis enzyme. In one embodiment, the glycan synthesis enzyme is a sugar activation enzyme, a sugar extension enzyme, a reagent regeneration enzyme, a sugar functionalization enzyme, a sugar support enzyme, or a sugar removal enzyme.

The invention provides materials, and in particular, magnetic materials, for producing glycans using one or more enzymes that are immobilized within bionanocatalysts (BNCs) which in turn are embedded within macroporous scaffolds to provide scaffolded bionanacatalyst (scaffolded BNCs). The scaffolded BNCs may be inside of modular flow cells for flow manufacturing. The modular flow cells may be mixed and matched for a highly customizable and highly efficient manufacturing process. The scaffolded BNCs are used in reactions for synthesizing glycans by contacting a glycan subunit or substrate with a scaffolded BNC to produce a second glycan, contacting a first glycan subunit and a second glycan subunit to produce a glycan comprising the first and second glycan subunits, or contacting a first glycan with a scaffolded BNC to produce a second glycan. Included are processes to modifying a glycan subunit and to connect glycans.

Scaffolded BNCs according to this invention are useful in the synthesis of glycans. Accordingly, this invention provides a method of making a glycan, comprising contacting a glycan substrate and an immobilized glycan synthesis enzyme, wherein the glycan synthesis enzyme is immobilized on a matrix, wherein the matrix comprises magnetic nanoparticles, wherein the nanoparticles are associated with a magnetic scaffold, to produce a glycan.

The invention also provides methods for making BNCs according to any of the methods disclosed herein, including but not limited to, those in Example 1, Example 2, Example 3, FIGS. 1-8, and any of the claims. The invention also provides methods for making macroporous matrix material according to any of the methods disclosed herein, including but not limited to, those in Example 1, Example 2, Example 3, FIGS. 1-8, and any of the claims.

In one embodiment, the magnetic macroporous material comprises a metal oxide or a metal oxide complex. In one embodiment, the metal oxide is strontium ferrite (SrFe₁₂O₁₉). In certain embodiments, the magnetic macroporous material is a metal oxide and consists essentially of, or consists of, metallic materials and does not include a polymer.

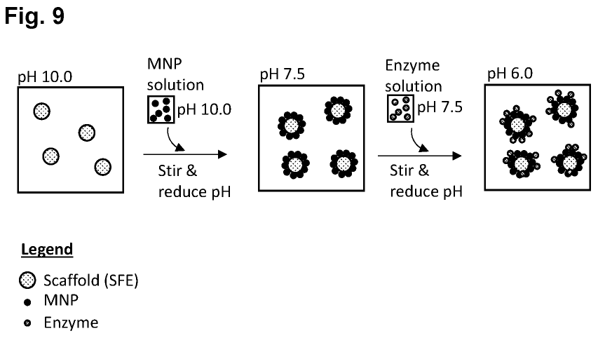

FIG. 9 depicts the enzyme immobilization scheme as employed for the embodiments of the invention exemplified in Examples 3b, 3c-A, 3c-B, 3c-C and 3c-D. In a first step, strontium ferrite (SFE) scaffold material is coated with magnetic nanoparticles (MNPs) by lowering the pH from 10.0 to 7.5. In a second step, the glycan synthesis enzymes are added to the product from the first step to the scaffolded BNCs. Type B scaffolded BNC compositions are made by this method.

In other embodiments of this invention, glycan synthesis enzymes are contacted with magnetic nanoparticles to form a bionanocatalyst (“BNC”) and then the BNCs are contacted with a magnetic scaffold material that is a magnetic microporous material. Type A scaffolded BNC compositions are made by this method. In certain embodiments, the magnetic scaffold material is strontium ferrite that has an average particle size of about 10 μm to about 120 μm, about 20 μm to about 40 μm, about 20 μm, or about 40 μm. In one embodiment the strontium ferrite is a spherical particle with a tight size distribution of an average particle diameter of either about 20 μm (S20) or about 40 μm (S40W; wrinkled). Strontium ferrite in accordance with this invention available upon request from Powdertech International. In certain embodiments, the strontium ferrite is S20 or is 40W from Powdertech International. Without being bound by theory, combining enzyme(s) and nanoparticles then adding that combination to the scaffold, the enzymes are entrapped (embedded) within the MNPs to provide Type A scaffolded BNCs. By adding enzyme(s) to nanoparticle coated scaffolds, a Type B scaffolded BNC composition is obtained, wherein the enzymes remain more exposed at the surface and are not buried as much as Type A. Accordingly, the glycan enzyme is magnetically immobilized within the mesopores or on their surface. As used herein, scaffold-MNP complex, scaffold-MNP matrix, and scaffold-MNP material each indicate the combination of a scaffold and a MNP according to this invention comprising, consisting essentially of, or consisting of, magnetite nanoparticles and a strontium ferrite matrix.

The invention also provides a process for preparing a scaffolded bionanocatalyst by combining a magnetic nanoparticle and a glycan synthesis enzyme to form a bionanocatalyst and then contacting the bionanocatalyst with a scaffold to obtain the scaffolded bionanocatalyst, and a process for preparing a scaffolded bionanocatalyst by combining a scaffold and a magnetic nanoparticle to form a scaffolded magnetic nanoparticle complex and then contacting the scaffolded magnetic nanoparticle complex with a glycan synthesis enzyme. Also provided is a scaffolded BNC made by either of these processes.

Accordingly, this invention provides a glycan synthesis enzyme scaffolded BNC made by the process of contacting strontium ferrite with magnetite nanoparticles to form a scaffold-MNP Complex and adding a glycan synthesis enzyme to the scaffold-MNP Complex to form the scaffolded BNC. Another embodiment provides a glycan synthesis enzyme scaffolded BNC made by the process of combining magnetite nanoparticles and a glycan synthesis enzyme to form a BNC and then contacting the BNC with a scaffold or matrix to form a scaffolded BNC. Without being bound by theory, the bionanocatalyst coats the magnetic microporous scaffold material.

One embodiment of this invention a scaffolded BNC composition comprises a self-assembled mesoporous aggregate of magnetic nanoparticles and a glycan synthesis enzyme and a magnetic microporous material. A scaffolded BNC according to this invention comprises a glycan synthesis enzyme immobilized on magnetic nanoparticles, wherein the magnetic nanoparticles coat a magnetic macroporous material. In certain embodiments, the scaffolded BNCs comprises a magnetic macroporous matrix material comprising self-assembled mesoporous aggregates of magnetic nanoparticles magnetically entrapping an immobilized glycan synthesis enzyme In certain embodiments, the scaffolded BNCs consists of, or consists essentially of, a magnetic macroporous matrix material comprising self-assembled mesoporous aggregates of magnetic nanoparticles magnetically entrapping an immobilized glycan synthesis enzyme. In any embodiments, the scaffolded BNC comprises any elementary enzyme module described herein or is in a system module.

The invention provides elementary enzyme modules and system enzyme modules comprising glycan synthesis enzyme for use in this invention. Elementary enzyme modules effect specific chemical transformation and are carefully designed and adapted to be used this invention. An elementary module is an enzyme system, that effects a chemical conversion from a compound A to a compound B. The elementary modules are designed to be combinable with each other to make glycans. An elementary enzyme module describes one or more than one glycan synthesis enzyme for use in embodiments of this invention. Elementary modules may comprise one or more enzymes and system modules may comprise one of more elementary modules. Each elementary module carries out one chemical transformation. System modules combine elementary modules to optionally provide multiple reaction steps to effect synthesis of a glycan. The system module may contain one enzyme or may contain two or more enzymes. Thus, a system module effects one or more chemical conversions. FIG. 1 and Table 1 describe modules of this invention.

The elementary modules are organized by six possible chemical transformations categories serving defined synthetic tasks in a synthesis, including multi-step synthesis, of glycans. EM-1 is a sugar activation module. EM-2 is a sugar extension module. EM-3 is a reagent regeneration module. EM-4 is a sugar functionalization module. EM-5 is a support enzyme module. EM-6 is a sugar removal module. See Table 1 herein. Each category has subcategories, sometimes multiple levels of subcategories, of glycan synthesis enzymes. All enzymes in Table 1 may be employed in embodiments of this invention. An elementary enzyme module describes one or more than one glycan synthesis enzyme for use in embodiments of this invention.

System enzyme modules (SM) comprise one elementary enzyme module (EM) or a combination of elementary enzyme modules (FIG. 1). System modules allow complex chemical reactions that interact with each other to be achieved in methods of this invention. In certain embodiments, the scaffolded bionanocatalysts are organized in system modules. In certain embodiments, the modules are organized in sequential modules. The invention provides any number of sequential modules. In one embodiment, the invention provides 1, 2, 3, 4, 5, 6, 7, 8, 9, or 10 sequential modules. A system module may be a reactor, flow reactor, continuous stirred-tank reactor, a semi-continuous reactor, or flow cell.

In certain embodiments, the system enzyme module is SM-1, SM-2, SM-3, SM-4, SM-5, SM-6, SM-7, SM-8, SM-9, SM-10, or SM-11. In another embodiment, it is SM-1, SM-4, or SM-6. In another embodiment, the module is SM-1, SM-4, and SM-6, optionally in a flow cell or a pack bed reactor.

Accordingly, in certain embodiments, the glycan synthesis enzyme in a scaffolded BNC of this invention is an enzyme or combination of enzymes of any of the elementary enzyme modules or system enzyme modules. In one embodiment, a module comprises a sugar activation enzyme EM-1, a sugar extension enzyme EM-2, a reagent regeneration enzyme EM-3, a functionalization enzyme EM-4, a support enzyme module EM-5, or a sugar removal module EM-6.

In another embodiment, the glycan synthesis enzyme is EM-1-1, wherein the glycan synthesis enzyme is a combination of one kinase and one nucleotide transferase, or a natural or synthetic fusion enzyme that integrates both functions into one enzyme; EM-1-2, an enzyme to produce activated sugars via oxidation from a structurally related activated sugar; EM-1-3, an enzyme to produce activated sugars via isomerization from a structurally related activated sugar; EM-1-4, a GlycoSynthetases to produce activated sugars from the respective un-activated sugar and cytidine triphosphate; EM-1-5, a kinase to produce a phosphorylated (1-P) monosaccharides; EM-2-1, a sugar extension enzyme phosphorylase that adds a phosphorylated (1-P) sugar donor to a sugar acceptor; EM-2-2, a GlycoSynthetase that adds an activated monosaccharide or oligosaccaride to a sugar acceptor; EM-2-3, Glycotransferase that adds an activated monosaccharide to a sugar acceptor; EM-3-1, a reagent regeneration enzyme for nucleotide regeneration; EM-3-2, a reagent regeneration subcategory to effect sugar nucleotide regeneration; EM-3-3, a cofactor regeneration enzyme for conversion of PAPS from PAP and NADH from NAD in connection with Sulfotransferase (EM-4-2) and UDP-Glc dehydrogenase (EM-1-2) enzyme systems; EM-4-1, phosphorylation (O-PO₃) enzyme (EC 2.7.8.17); EM-4-2, Sulfation (0-S03) enzyme; EM-5-1, pyrophosphorylase (EC 3.6.1.1) that converts pyrophosphate (PPi) to monophosphate (Pi); EM-6-1, fucosidase; EM-6-2, galactosidase; or EM-6-3, sialidase.

In another embodiment, the glycan synthesis enzyme is EM-1-1-1, a combination of galactokinase (GalK, EC 2.7.1.6) and galactose-1-phosphate uridyltransferase (Gal-1-phosphate-UDP T, EC 2.7.7.64) that catalyzes the conversion of Galactose to UDP-Galactose; EM-1-1-2, a combination of N-acetylglucosamine kinase (G1cNAcK, EC 2.7.1.162) and N-acetylglucosamine-1-phosphate-uridyltransferase (GlmU, EC 2.7.7.23) that catalyzes the conversion of GlcNAc to UDP-GlcNAc; EM-1-1-3, combination of fucokinase (fucK, EC 2.7.1.52) and fucose-1-phosphate-guanylyltransferase (Fuc-1P-GDP T, EC 2.7.7.30) that catalyzes the conversion of L-fucose to GDP-fucose; EM-1-1-4, a combination of glucuronokinase (GlcA K, EC 2.7.1.43) and glucurono-1-phosphate-uridyltransferase (GlcA-1-phosphate-UDP T, EC 2.7.7.44) that catalyzes the conversion of GlcA to UDP-GlcA; EM-1-1-5, a combination of glucokinase (Glc K, EC 2.7.1.2) and glucose-1-phosphate-uridyltransferase (Glc-1-phosphate-UDP T, EC 2.7.7.9) that catalyzes the conversion of Glc to UDP-Glc; EM-1-1-6, a combination of mannokinase (Man K, EC 2.7.1.7) and mannose-1-phosphate-guanyltransferase (Man-1-phosphate-GDP T, EC 2.7.7.13, 2.7.7.22) that catalyzes the conversion of Man to GDP-Man; EM-1-1-7, a combination of rhamnokinase (Rha K, EC 2.7.1.5) and rhamnose-1-phosphate-uridyltransferase (Rha-1-phosphate-UDP T) that catalyzes the conversion of Rha to UDP-Rha; EM-1-1-8, a natural fusion enzyme (bifunctional fucokinase/L-fucose-1-P-guanylyltransferase=FKP, EC 2.7.1.52/2.7.7.30) that catalyzes the conversion of L-fucose to GDP-fucose; EM-1-1-9, a synthetic fusion enzyme combining N-acetylglucosamine kinase (G1cNAcK, EC 2.7.1.162) and N-acetylglucosamine-1-phosphate-uridyltransferase (GlmU, EC 2.7.7.23) that catalyzes the conversion of GlcNAc to UDP-GlcNAc; EM-1-2-1, an oxidase (UDP-Glc-6-dehydrogenase, EC 1.1.1.22) that catalyzes the conversion of UDP-Glc to UDP-GlcA (UDP-Glucuronic acid); EM-1-3-1, an epimerase (UDP-Gal-4-epimerase, EC 5.1.3.2) that catalyzes the conversion of UDP-Glc to UDP-Gal; EM-1-4-1, a GlycoSynthetase (CMP-sialic acid synthetase=CSS, EC 2.7.7.43) that catalyzes the conversion of neuraminic acid (Neu5Ac) and cytidine triphosphate (CTP) to CMP-Neu5Ac; EM-1-4-2, a GlycoSynthetase (3-deoxy-manno-octulosonate cytidyl transferase synthetase=CMP-KDO synthetase=CKS, EC 2.7.7.38) that catalyzes the conversion of 3-deoxy-manno-octulosonate (KDO) and cytidine triphosphate (CTP) to CMP-KDO; EM-1-5-1, a kinase (galactokinase, EC 2.7.1.6) that catalyzes the conversion of galactose (Gal) to galactose-1-phosphate (Gal-1-P); EM-1-5-2, a kinase (glucokinase, EC 2.7.1.2) that catalyzes the conversion of glucose (Glc) to glucose-1-phosphate (Glc-1-P); EM-2-1-1, phosphorylase (1,3-β-galactosyl-N-acetylhexosamine phosphorylase=galacto-N-biose phosphorylase=GalHexNAcP, EC 2.4.1.211) that catalyzes the conversion of Gal-1P (sugar donor) to GlcNAc with a β-1,3 linkage; EM-2-2-1, a fucosidase (EC 3.2.1.51) that catalyzes the transfer of activated L-fucose (nitrophenylated L-fucose or fluoronated L-fucose) onto either Galactose, Glucose, or GlcNAc; EM-2-2-2, a neuraminidase (sialidase, EC 3.2.1.18) that catalyzes the transfer of activated neuraminic acid (4MU-Neu5Ac, colominic acid, fetuin or sialylconjugates) onto either Galactose, GlcNAc or neuraminic acid; EM-2-2-3, a β-N-acetylhexosaminidase (β-N-acetylglucosaminidase, EC 3.2.1.52) that catalyzes the transfer of an activated GlcNAc (oxazolinated GlcNAc, nitrophenylated GlcNAc or fluorinated GlcNAc) to Galactose, GlcA, Mannose, GalNAc or GlcNAc; EM-2-2-4, a β-N-acetylgalactosaminidase (EC 3.2.1.53) that catalyzes the transfer of an activated GalNAc (oxazolinated GalNAc, nitrophenylated GalNAc or fluorinated GalNAc) to Galactose, GlcA or IdoA; EM-2-2-5, an α or β-galactosidase (EC 3.2.1.22, 3.2.1.23) that catalyzes the transfer of activated Gal (nitrophenylated Gal, fluorinated Gal or lactose) to Galactose, Glucose, GlcNAc or GalNAc; EM-2-2-6, a β-glucuronidase (EC 3.2.1.31) that catalyzes the transfer of an activated glucuronic acid GlcA (nitrophenylated GlcA or fluorinated GlcA) to Galactose, Glucose GalNAc or GlcNAc; EM-2-2-7, an α or β-glucosidase (EC 3.2.1.20, 3.2.1.21) that catalyzes the transfer of activated Glucose Glc (nitrophenylated Glc or fluorinated Glc) to Mannose, GlcNAc or Gal; EM-2-2-8, an α or β-mannosidase (EC 3.2.1.24, 3.2.1.25) that catalyzes the transfer of activated Mannose Man (nitrophenylated Man or fluorinated Man) to Man or GlcNAc; EM-2-2-9, an β-xylosidase (EC 3.2.1.37) that catalyzes the transfer of activated Xylose Xyl (nitrophenylated Xyl or fluorinated Xyl) to xylose, mannose, galactose or glcNAc; EM-2-2-10, an endo specific glycosynthetase that transfers sugar oligosaccharides to acceptor glycans; EM-2-3-1, a fucosyltransferase that catalyzes the transfer of GDP-fucose (GDP-Fuc) onto either Galactose, Glucose or GlcNAc; EM-2-3-2, a sialyltransferase that catalyzes the transfer of CMP-neuraminic acid (CMP-Neu5Ac) onto either GlcNAc, galactose or Neu5Ac; EM-2-3-3, an N-acetylglucosaminyltransferase that catalyzes the transfer of UDP-N-acetylglucosamine (UDP-GlcNAc) to galactose, mannose and GlcNAc; EM-2-3-4, an N-acetylgalactosyltransferase that catalyzes the transfer of an activated UDP-N-acetylgalactosamine (UDP-GalNAc) to Galactose; EM-2-3-5, a galactosyltransferase that catalyzes the transfer of activated UDP-Galactose (UDP-Gal) to Glucose and GlcNAc; EM-2-3-6, a glucuronic acid transferase that catalyzes the transfer of an activated UDP-glucuronic acid (UDP-GlcA) to Galactose and GlcNAc; EM-2-3-7, a glucosyltransferase that catalyzes the transfer of activated UDP-Glucose (UDP-Glc) to GlcNAc, mannose, glucose or Gal; EM-2-3-8, a mannosyltransferase that catalyzes the transfer of activated UDP-Mannose (UDP-Man) GlcNAc or Man; EM-2-3-9, a rhamnosyltransferase that catalyzes the transfer of activated UDP-Rhamnose (UDP-Rham) to Gal or GlcNAc; EM-2-3-10, a xylosyltransferase that catalyzes the transfer of activated UDP-Xylose (UDP-Xyl) to xylose or GlcNAc; EM-3-1-1, a nucleotide regeneration enzyme to convert of UDP to UTP; EM-3-1-2, a nucleotide regeneration enzyme to convert GDP to GTP; EM-3-1-3, a nucleotide regeneration enzyme that converts CMP to CTP using a one-enzyme or two-enzyme system; EM-3-1-4, a nucleotide regeneration category to convert ADP to ATP and optionally coupled with EM-3-1-1-1, EM-3-1-1-2, EM-3-1-2-1, EM-3-1-3-2, EM-3-1-3-3, EM-3-1-3-4, and EM-3-1-3-5; EM-3-2-1, a nucleotide regeneration enzyme to convert UDP to UDP-Glc; EM-3-3-1, Aryl sulfotransferase IV (EC 2.8.2.9); EM-3-3-2, oxidase to convert NAD (Nicotinamide adenine dinucleotide) to NADH (1,4-Dihydronicotinamide adenine dinucleotide); EM-4-1-1, a GNPTG form Homo sapiens; EM-4-2-1, carbohydrate sulfotransferase, human enzyme; EM-4-2-2, Galactose-3-O-sulfotransferase, human enzyme; EM-4-2-3, Heparan sulfate O-sulfotransferase, human enzyme; EM-4-2-4, N-deacetylase/N-sulfotransferase, human enzyme (EC 2.8.2.8); EM-5-1-1, PmPpa from Pasteurella multocida; EM-6-1-1, 1,2-α-L-fucosidase (EC 3.2.1.63); EM-6-1-2, 1,3-α-L-fucosidase (EC 3.2.1.111); EM-6-1-3, 1,6-α-L-fucosidase (EC 3.2.1.127); EM-6-1-4, α-L-fucosidase (EC 3.2.1.51); EM-6-2-1, α-galactosidase (EC 3.2.1.22), EM-6-2-2, β-galactosidase (EC 3.2.1.23); or EM-6-3-1, or exo-α-sialidase (EC 3.2.1.18).

In another embodiment, the glycan synthesis enzyme is EM-1-1-1-1, BiGalK/BiUSP from Bifidobacterium infantis; EM-1-1-2-1, BiNahK from Bifidobacterium infantis, HmG1mU from Helicobacter mustelae; EM-1-1-2-2, BiNahK from Bifidobacterium infantis, and CjG1mU from Campylobacter jejuni; EM-1-1-8-1, BfFKP from Bacteroides fragilis; EM-1-1-9-1, BINahK-EcGlmU from Bifidobacterium infantis and Escherichia coli; EM-1-3-1-1, EcGalE from Escherichia coli; EM-1-3-1-2, StGalE from Streptococcus thermophilus; EM-1-4-1-1, NmCSS from Neisseria meningitides; EM-1-5-1-1. BiGalK from Bifidobacterium infantis, EM-2-1-1-1, BiGalHexNAcP from Bifidobacterium infantis; EM-2-2-1-1, α-1,2-fucosidase (EC 3.2.1.63); EM-2-2-1-2, α-1,3-fucosidase (EC 3.2.1.111); EM-2-2-1-3, α-1,4-fucosidase; EM-2-2-2-1, α-2,3-neuraminidase; EM-2-2-2-2, α-2,6-neuraminidase; EM-2-2-2-3, α-2,8-neuraminidase; EM-2-2-3-1, β-1,3-N-acetylglucosaminidase; EM-2-2-4-1, β-1,4-N-acetylgalactosaminidase; EM-2-2-5-1, α-1,2-galactosidase; EM-2-2-5-2, α-1,3-galactosidase; EM-2-2-5-3, α-1,4-galactosidase; EM-2-2-5-4, α-1,6-galactosidase; EM-2-2-5-5, β-1,3-galactosidase; EM-2-2-5-6, β-1,4-galactosidase; EM-2-2-5-7, β-1,6-galactosidase; EM-2-2-6-1, β-1,3-glucuronidase; EM-2-2-7-1, β-1,3-glucosidase; EM-2-2-7-2, β-1,4-glucosidase; EM-2-2-7-3, β-1,6-glucosidase; EM-2-2-7-4, α-1,4-glucosidase; EM-2-2-8-1, α-1,2-mannosidase; EM-2-2-8-2, α-1,3-mannosidase; EM-2-2-8-3, β-1,3-mannosidase; EM-2-2-8-4, β-1,4-mannosidase; EM-2-2-9-1, β-1,4-xylosidase; EM-2-2-10-1, arabinogalactan endo-β-1,4-galactanase (EC 3.2.1.89); EM-2-2-10-2, mannosyl-glycoprotein endo-β-N-acetylglucosaminidase (EC 3.2.1.96); EM-2-2-10-3, endo-α-N-acetylgalactosaminidase (EC 3.2.1.97); EM-2-2-10-4, blood-group-substance endo-1,4-β-galactosidase (EC 3.2.1.102); EM-2-2-10-5, keratan-sulfate endo-1,4-β-galactosidase (EC 3.2.1.103); EM-2-2-10-6, glycoprotein endo-α-1,2-mannosidase (EC 3.2.1.130); EM-2-2-10-7, lacto-N-biosidase (EC 3.2.1.140); EM-2-2-10-8, mannosylglycoprotein endo-β-mannosidase (EC 3.2.1.152); EM-2-3-1-1, α-1,2-fucosyltransferase (EC 2.4.1.69); EM-2-3-1-2, α-1,3-fucosyltransferase (EC 2.4.1.152, EC 2.4.1.214); EM-2-3-1-3, α-1,4-fucosyltransferase (EC 2.4.1.65); EM-2-3-1-4, α-1,6-fucosyltransferase (EC 2.4.1.68); EM-2-3-2-1, α-2,3-sialyltransferase (EC 2.4.99.4, EC 2.4.99.6, EC 2.4.99.7, EC 2.4.99.9); EM-2-3-2-2, α-2,6-sialyltransferase (EC 2.4.99.1, EC 2.4.99.3); EM-2-3-2-3, α-2,8-sialyltransferase (EC 2.4.99.8); EM-2-3-3-1, β-1,3-N-acetylglucosaminyltransferase (EC 2.4.1.79, EC 2.4.1.149, EC 2.4.1.222); EM-2-3-3-2, α-1,4-N-acetylglucosaminyltransferase (EC 2.4.1.223, EC 2.4.1.224); EM-2-3-3-3, β-1,2-N-acetylglucosaminyltransferase (EC 2.4.1.101, EC 2.4.1.143); EM-2-3-3-4, β-1,4-N-acetylglucosaminyltransferase (EC 2.4.1.144, EC 2.4.1.212); EM-2-3-3-5, β-1,6-N-acetylglucosaminyltransferase (EC 2.4.1.102, EC 2.4.1.150, EC 2.4.1.155); EM-2-3-4-1, α-1,3-N-acetylgalactosyltransferase (EC 2.4.1.40); EM-2-3-4-2, β-1,4-N-acetylgalactosyltransferase (EC 2.4.1.174, EC 2.4.1.175); EM-2-3-5-1, β-1,3-galactosyltransferase (EC 2.4.1.122, EC 2.4.1.134); EM-2-3-5-2, β-1,4-galactosyltransferase (EC 2.4.1.22, EC 2.4.1.38, EC 2.4.1.90, EC 2.4.1.133); EM-2-3-5-3, α-1,3-galactosyltransferase (EC 2.4.1.37); EM-2-3-5-4, α-1,6-galactosyltransferase (EC 2.4.1.241); EM-2-3-6-1, β-1,3-glucuronic acid transferase (EC 2.4.1.135, EC 2.4.1.212, EC 2.4.1.226); EM-2-3-6-2, β-1,4-glucuronic acid transferase (EC 2.4.1.226); EM-2-3-7-1, β-1,2-glucosyltransferase (EC 2.4.1.208); EM-2-3-7-2, 0-1,3-glucosyltransferase (EC 2.4.1.305); EM-2-3-7-3, α-1,3-glucosyltransferase (EC 2.4.1.256, EC 2.4.1.265, EC 2.4.1.267); EM-2-3-7-4, α-1,4-glucosyltransferase (EC 2.4.1.374); EM-2-3-8-1, α-1,2-mannosyltransferase (EC 2.4.1.131, EC 2.4.1.259, EC 2.4.1.260, EC 2.4.1.270); EM-2-3-8-2, α-1,3-mannosyltransferase (EC 2.4.1.132, EC 2.4.1.252, EC 2.4.1.258); EM-2-3-8-3, β-1,4-mannosyltransferase (EC 2.4.1.142, EC 2.4.1.251); EM-2-3-8-4, α-1,6-mannosyltransferase (EC 2.4.1.257, EC 2.4.1.260); EM-2-3-9-1, α-1,3-rhamnosyltransferase (EC 2.4.1.159, EC 2.4.1.289); EM-2-3-9-2, α-1,4-rhamnosyltransferase (EC 2.4.1.3751); EM-2-3-10-1, β-1,2-xylosyltransferase (EC 2.4.2.38); EM-2-3-10-2, α-1,6-xylosyltransferase (EC 2.4.2.39); EM-3-1-1-1, Uridine-diphosphate kinase (EC 2.7.4.6) converts UDP to UTP coupled to stoichiometric conversion of ATP to ADP; EM-3-1-1-2, nucleoside-diphosphate kinase (EC 2.7.4.6) converts UDP to UTP coupled to stoichiometric conversion of ATP to ADP; EM-3-1-1-3, pyruvate kinase (EC 2.7.1.40) converts UDP to UTP coupled to stoichiometric conversion of phosphoenolpyruvate (PEP) to pyruvate; EM-3-1-1-4, acetate kinase, AcK (EC 2.7.4.1) converts UDP to UTP coupled to stoichiometric conversion of acetyl phosphate (AcPi) to acetate (Ac); EM-3-1-2-1, nucleoside-diphosphate kinase (EC 2.7.4.6) converts GDP to GTP coupled to stoichiometric conversion of ATP to ADP; EM-3-1-2-2, pyruvate kinase (EC 2.7.1.40) converts GDP to GTP coupled to stoichiometric conversion of phosphoenolpyruvate (PEP) to pyruvate; EM-3-1-2-3, acetate kinase, AcK (EC 2.7.4.1) converting GDP to GTP coupled to stoichiometric conversion of acetyl phosphate (AcPi) to acetate (Ac); EM-3-1-3-1, cytidine-monophosphate kinase, CMPK (EC 2.7.4.4) and acetate kinase, AcK (EC 2.7.4.1) that converts CMP to CTP coupled to stoichiometric conversion of acetyl phosphate (AcPi) to acetate (Ac); EM-3-1-3-2, cytidine-monophosphate kinase, CMPK (EC 2.7.4.4) and nucleoside-diphosphate kinase, NDPK (EC 2.7.4.6) that converts CMP to CTP coupled to stoichiometric conversion of two ATPs to two ADPs; EM-3-1-3-3, cytidine-monophosphate kinase, CMPK (EC 2.7.4.4) and Cytidine-diphosphate kinase, CDK (EC 2.7.4.6); EM-3-1-3-4, nucleoside-monophosphate kinase, NMK (EC 2.7.4.4) and cytidine-diphosphate kinase, CDK (EC 2.7.4.6) that converts CMP to CTP coupled to stoichiometric conversion of two ATPs to two ADPs; EM-3-1-3-5, nucleoside-monophosphate kinase, NMK (EC 2.7.4.4) and nucleoside-diphosphate kinase, NDPK (EC 2.7.4.6)) that converts CMP to CTP coupled to stoichiometric conversion of two ATPs to two ADPs; EM-3-1-4-1, acetate kinase, AcK (EC 2.7.4.1) that converts ADP to ATP coupled to stoichiometric conversion of acetyl phosphate (AcPi) to acetate (Ac); EM-3-1-4-2, pyruvate kinase (EC 2.7.1.40) that converts ADP to ATP coupled to stoichiometric conversion of phosphoenolpyruvate (PEP) to pyruvate; EM-3-1-4-3, polyphosphate kinase, PpK (EC 2.7.4.1) converts ADP to ATP coupled to stoichiometric conversion of polyphosphate (Pₙ) to acetate Pₙ₋₁; EM-3-2-1-1, sucrose Synthase (EC 2.4.1.13) that converts UDP and sucrose to UDP-Glc and the byproduct fructose; EM-3-3-2-1, lactate dehydrogenase (EC 1.1.1.27) that converts NAD to NADH coupled to the stoichiometric conversion of lactate to pyruvate; EM-3-3-2-2, xylose reductase (EC 1.1.1.307) that converts NAD to NADH coupled to the stoichiometric conversion of xylitol to D-xylose; EM-4-2-1-1, carbohydrate sulfotransferase 1 (CHST1, EC 2.8.2.21); EM-4-2-1-2, carbohydrate sulfotransferase 2 (CHST2, EC 2.8.2.-); EM-4-2-1-3, carbohydrate sulfotransferase 3 (CHST3, EC 2.8.2.17); EM-4-2-1-4, carbohydrate sulfotransferase 4 (CHST4, EC 2.8.2.-); EM-4-2-1-5, carbohydrate sulfotransferase 5 (CHST5, EC 2.8.2.-); EM-4-2-1-6, carbohydrate sulfotransferase 6 (CHST6, EC 2.8.2.-; keratan sulfotransferase 2.8.2.21); EM-4-2-1-7, carbohydrate sulfotransferase 7 (CHST7, EC 2.8.2.17); EM-4-2-1-8, carbohydrate sulfotransferase 8 (CHST8, EC 2.8.2.-); EM-4-2-1-9, carbohydrate sulfotransferase 9 (CHST9, EC 2.8.2.-); EM-4-2-1-10, carbohydrate sulfotransferase 10 (CHST10, EC 2.8.2.-); EM-4-2-1-11, carbohydrate sulfotransferase 11 (CHST11, EC 2.8.2.5); EM-4-2-1-12, carbohydrate sulfotransferase 12 (CHST12, EC 2.8.2.5); EM-4-2-1-13, carbohydrate sulfotransferase 13 (CHST13, EC 2.8.2.5); EM-4-2-1-14, carbohydrate sulfotransferase 14 (dermatan 4-sulfotransferase CHST14, EC 2.8.2.35); EM-4-2-1-15, carbohydrate sulfotransferase 15 (CHST15, EC 2.8.2.33); EM-4-2-2-1, galactose-3-O-sulfotransferase (GAL3ST1, EC 2.8.2.11); EM-4-2-2-2, galactose-3-O-sulfotransferase (GAL3ST2, EC 2.8.2.-); EM-4-2-2-3, galactose-3-O-sulfotransferase (GAL3ST3, EC 2.8.2.-); EM-4-2-2-4, galactose-3-O-sulfotransferase (GAL3ST4, EC 2.8.2.-); EM-4-2-3-1, heparan sulfate 2-O-sulfotransferase 1 (HS2ST1, EC 2.8.2.-); EM-4-2-3-2, heparan sulfate 3-O-sulfotransferase 1 (HS3ST1, EC 2.8.2.23); EM-4-2-3-3, heparan sulfate 3-O-sulfotransferase 2 (HS3ST2, EC 2.8.2.29); EM-4-2-3-4, heparan sulfate 3-O-sulfotransferase 2A1 (HS3ST3A1, EC 2.8.2.30); EM-4-2-3-5, heparan sulfate 3-O-sulfotransferase 3B1 (HS3ST3B1, EC 2.8.2.30); EM-4-2-3-6, heparan sulfate 3-O-sulfotransferase 4 (HS3ST4, EC 2.8.2.23); EM-4-2-3-7, heparan sulfate 3-O-sulfotransferase 5 (HS3ST5, EC 2.8.2.23); EM-4-2-3-8, heparan sulfate 3-O-sulfotransferase 6 (HS3ST6, EC 2.8.2.23); EM-4-2-3-9, heparan sulfate 6-O-sulfotransferase 1 (HS6ST1, EC 2.8.2.-); EM-4-2-3-10, heparan sulfate 6-O-sulfotransferase 2 (HS6ST2, EC 2.8.2.-); EM-4-2-3-11, heparan sulfate 6-O-sulfotransferase 3 (HS6ST3, EC 2.8.2.-); EM-4-2-3-12, (heparan sulfate)-glucosamine 3-sulfotransferase 1 (EC 2.8.2.23); EM-4-2-3-13, (heparan sulfate)-glucosamine 3-sulfotransferase 2 (EC 2.8.2.29); EM-4-2-3-14, (heparan sulfate)-glucosamine 3-sulfotransferase 3 (EC 2.8.2.30); EM-4-2-4-1, N-deacetylase/N-sulfotransferase 1 (NDST1); EM-4-2-4-2, N-deacetylase/N-sulfotransferase 2 (NDST2); EM-4-2-4-3, N-deacetylase/N-sulfotransferase 3 (NDST3); EM-4-2-4-4, N-deacetylase/N-sulfotransferase 4 (NDST4); EM-4-2-4-5, (heparan sulfate)-glucosamine N-sulfotransferase; EM-6-1-4-1, α-Fucosidase from Thermotoga maritima; EM-6-1-4-2, α-(1-2,3,4,6)-Fucosidase from Homo sapiens; EM-6-2-2-1, β-galactosidase from Aspergillus niger; EM-6-2-2-2, 0-galactosidase from Escherichia coli; EM-6-2-2-3, β-galactosidase from Aspergillus oryzae; EM-6-2-2-4, β-galactosidase from Kluyveromyces lactis; EM-6-3-1-1, exo-α-sialidase (Salmonella typhimurium); or EM-6-3-1-2, exo-α-sialidase (Clostridium perfringens).

In another embodiment, the glycan synthesis enzyme is EM-2-2-3-1-1, Bbh1 from Bifidobacterium bifidum; EM-2-2-10-7-1, LnbB from Bifidobacterium bifidum; EM-2-3-1-1-1, Te2FT from Thermosynechococcus elongatus; EM-2-3-1-1-2, WbgL from Escherichia coli; EM-2-3-1-1-3, HmFucT from Helicobacter mustelae; EM-2-3-1-1-4, FUT1 from Homo sapiens; EM-2-3-1-1-5, FUT2 from Homo sapiens; EM-2-3-1-2-1, HpFucT from Helicobacter pylori; EM-2-3-1-2-2, Bf1,3FT from Bacteroides fragilis; EM-2-3-1-2-3, Hp3/4FT from Helicobacter pylori; EM-2-3-1-2-4, FUT3 from Homo sapiens; EM-2-3-1-2-5, FUT4 from Homo sapiens; EM-2-3-1-2-6, FUT5 from Homo sapiens; EM-2-3-1-2-7, FUT6 from Homo sapiens; EM-2-3-1-2-8, FUT7 from Homo sapiens; EM-2-3-1-2-9, FUT9 from Homo sapiens; EM-2-3-1-2-10, FUT11 from Homo sapiens; EM-2-3-1-3-1, Hp3/4FT from Helicobacter pylori; EM-2-3-1-3-2, FUT2 from Homo sapiens; EM-2-3-1-4-1, FUT8 from Homo sapiens; EM-2-3-2-1-1, PmST1 (wild type and M144D mutant) from Pasteurella multocida; EM-2-3-2-1-2, NmST1-NmCSS fusion from Neisseria meningitidis; EM-2-3-2-1-3, ST3GAL1 from Homo sapiens; EM-2-3-2-1-4, ST3GAL2 from Homo sapiens; EM-2-3-2-1-5, ST3GAL3 from Homo sapiens; EM-2-3-2-1-6, ST3GAL4 from Homo sapiens; EM-2-3-2-1-7, ST3GAL5 from Homo sapiens; EM-2-3-2-1-8, ST3GAL6 from Homo sapiens; EM-2-3-2-2-1, Pd26ST from Photobacterium damsel; EM-2-3-2-2-2, ST6GAL1 from Homo sapiens; EM-2-3-2-2-3, ST6GAL2 from Homo sapiens; EM-2-3-2-2-4, ST6GALNAC1 from Homo sapiens; EM-2-3-2-2-5, ST6GALNAC2 from Homo sapiens; EM-2-3-2-2-6, ST6GALNAC3 from Homo sapiens; EM-2-3-2-2-7, ST6GALNAC4 from Homo sapiens; EM-2-3-2-2-8, ST6GALNAC5 from Homo sapiens; EM-2-3-2-2-9, ST6GALNAC6 from Homo sapiens; EM-2-3-2-3-1, α-2,3/8-sialyltransferase from Campylobacter jejuni; EM-2-3-2-3-2, ST8SIA1 from Homo sapiens; EM-2-3-2-3-3, ST8SIA2 from Homo sapiens; EM-2-3-2-3-4, ST8SIA3 from Homo sapiens; EM-2-3-2-3-5, ST8SIA4 from Homo sapiens; EM-2-3-2-3-6, ST8SIA5 from Homo sapiens; EM-2-3-3-1-1, HpLgtA form Helicobacter pylori; EM-2-3-3-1-2, NmLgtA form Neisseria meningitidis; EM-2-3-3-1-3, HP1105 from Helicobacter pylori; EM-2-3-3-1-4, B3GNT2 from Homo sapiens; EM-2-3-3-1-5, B3GNT3 from Homo sapiens; EM-2-3-3-1-6, B3GNT4 from Homo sapiens; EM-2-3-3-1-7, B3GNT7 from Homo sapiens; EM-2-3-3-1-8, B3GNT8 from Homo sapiens; EM-2-3-3-1-9, B3GNT9 from Homo sapiens; EM-2-3-3-3-1, MGAT1 (G1cNAcT-I) from Homo sapiens; EM-2-3-3-3-2, MGAT2 (G1cNAcT-II) from Homo sapiens; EM-2-3-3-4-1, MGAT3 (G1cNAcT-III) from Homo sapiens; EM-2-3-3-4-2, MGAT4A (G1cNAcT-IV) from Homo sapiens; EM-2-3-3-4-3, MGAT4B (G1cNAcT-IV) from Homo sapiens; EM-2-3-3-4-4, MGAT4C (G1cNAcT-IV) from Homo sapiens; EM-2-3-3-5-1, GCNT2A from Homo sapiens; EM-2-3-3-5-2, GCNT2B from Homo sapiens; EM-2-3-3-5-3, GCNT2C from Homo sapiens; EM-2-3-3-5-4, GCNT3 from Homo sapiens; EM-2-3-3-5-5, GCNT4 from Homo sapiens; EM-2-3-3-5-6, MGAT5 (G1cNACT-V) from Homo sapiens; EM-2-3-4-1-1, ABO from Homo sapiens; EM-2-3-4-1-2, BgtA from Helicobacter mustelae; EM-2-3-4-2-1, B4GALNT3 from Homo sapiens; EM-2-3-4-2-2, B4GALNT4 from Homo sapiens; EM-2-3-5-1-1, Cvf33Ga1T from Chromobacterium violaceum; EM-2-3-5-1-2, WbgO from Escherichia coli; EM-2-3-5-1-3, CgtB from Campylobacter jejuni; EM-2-3-5-1-4, B3GALT1 from Homo sapiens; EM-2-3-5-1-5, B3GALT2 from Homo sapiens; EM-2-3-5-1-6, B3GALT4 from Homo sapiens; EM-2-3-5-1-7, B3GALT5 from Homo sapiens; EM-2-3-5-2-1, NmLgtB from Neisseria meningitidis; EM-2-3-5-2-2, NmLgtB-StGalE from Neisseria meningitidis and Streptococcus thermophilus; EM-2-3-5-2-3, HpLgtB from Helicobacter pylori; EM-2-3-5-2-4, B4GALT1 from Homo sapiens; EM-2-3-5-2-5, B4GALT2 from Homo sapiens; EM-2-3-5-2-6, B4GALT3 from Homo sapiens; EM-2-3-5-2-7, B4GALT4 from Homo sapiens; EM-2-3-5-2-8, B4GALT5 from Homo sapiens; EM-2-3-5-2-9, B4GALT6 from Homo sapiens; EM-2-3-5-2-10, B4GALT7 from Homo sapiens; EM-2-3-5-3-1, GTB (human proteins) synthetic gene expressed in E. Coli; EM-2-3-5-3-2, WbnL from E. Coli; EM-2-3-6-2-1, B4GAT1 from Homo sapiens; EM-3-1-1-2-1, nucleoside-diphosphate kinase, Saccharomyces cerevisiae; EM-3-1-1-2-2, nucleoside-diphosphate kinase, bovine; EM-3-1-1-4-1. Acetate kinase, Escherichia coli; EM-3-1-1-4-2, Acetate kinase, Clostridium acetobutylicum; EM-3-1-2-1-1, nucleoside-diphosphate kinase, Saccharomyces cerevisiae; EM-3-1-2-1-2, nucleoside-diphosphate kinase, bovine; EM-3-1-2-3-1, acetate kinase, Escherichia coli; EM-3-1-2-3-2, Acetate kinase, Clostridium acetobutylicum; EM-3-1-3-1-1, Acetate kinase, Escherichia coli; EM-3-1-3-1-2, acetate kinase, Clostridium acetobutylicum; EM-3-1-3-2-1, Nucleoside-diphosphate kinase, Saccharomyces cerevisiae; EM-3-1-3-2-2. Nucleoside-diphosphate kinase, bovine; EM-3-1-4-1-1, acetate kinase, Escherichia coli; EM-3-1-4-1-2, acetate kinase, Clostridium acetobutylicum; or EM-3-2-1-1-1, Sucrose Synthase (SUS), Arabidopsis.

Certain elementary modules are depicted in FIG. 1. The legend describes the identity of sugar symbols and sugar shorthand pertaining to all figures and body of the text, including description and claims. Accordingly, EM1.1A is EM-1-1-8-1. EM-1-3A is EM-1-3-1-2. EM-1-4A is EM-1-4-1-1. EM-2-2A is EM-2-2-3-1-1. EM-2-3A is EM-2-3-1-1-1. EM-2-3B is EM-2-3-1-1-2. EM-2-3C is EM-2-3-1-2-2. EM-2-3D is EM-2-3-2-1-1. EM-2-3E is EM-2-3-2-2-2. EM-2-3F is EM-2-3-2-2-8. EM-2-3G is EM-2-3-5-1-1. EM-2-3H is EM-2-3-5-2-1. EM-3-1A is EM-3-1-2-2. EM-3-1B is EM-3-1-3-2. EM-3-2A is EM-3-2-1-1-1. EM-5-1A is EM-5-1-1. EM6-3A is EM-6-3-1. https://www.glycoforum.grjp/article/22A2.html

Module EM-1-1A (EM-1-1-8-1) is depicted in FIG. 1A. The glycan synthesis enzyme is BfFKP (bifunctional L-fucokinase/GDP-L-Fuc pyrophosphorylase from Bacteroides fragilis. BfFKP converts L-fucose, ATP and GTP to L-fucose-GDP, ADP and inorganic diphosphate PPi. The module is termed an activation elementary module that activates L-fucose for functionalization (fucosylation) of a sugar moiety. Table 2 and Example 4 detail the enzyme expression. In certain embodiments, a BNC of this invention comprises BfFKP.

Module EM-1-3A (EM-1-3-1-2) is depicted in FIG. 1B. The glycan synthesis enzyme is StGalE (UDP-4′-gal-epimerase=StGalE; StGalE from Streptococcus thermophilus; enzyme is expressed) that converts Glc-UDP to Gal-UDP and then transfers Gal to GlcNAc while expelling UDP. The module is termed an activation elementary module that produce UDP-Gal for the elongation of a glycan or HMO core structure. In certain embodiments, a BNC of this invention comprises StGalE. See Example 4.

Module EM-1-4A (EM-1-4-1-1) is depicted in FIG. 1C. The glycan synthesis enzyme is NmCSS (CMP—sialic acid synthetase from Neisseria meningitidis; for enzyme expression see Ex. 4) that converts Neu5Ac and CTP to Neu5Ac-CMP and inorganic diphosphate PPi. The module is termed an activation elementary module that activate Neu5Ac for functionalization (sialylation) of a sugar moiety. In certain embodiments, a BNC of this invention comprises NmCSS. Example 4B provides a preparation of the enzyme. Examples 1-3 describe how to prepare BNCs and scaffolded BNCs comprising NmCSS, and Examples 1-3 describe how to use the scaffolded BNCs to synthesize glycans.

Module EM-2-2A (EM-2-2-3-1-1) is depicted in FIG. 1D. The glycan synthesis enzyme is Bbh1 (β-N-acetylglucosaminidase from Bifidobacterium bifidum; for enzyme expression see Ex. 4) that transfers GlcNAc from chemically synthesized GlcNAc-oxazoline to Gal with an β-1,3 linkage. The module is termed an extension elementary module that elongates a glycan, including but not limited to, a HMO core structure. In certain embodiments, a BNC of this invention comprises Bbh1. Example 4 describes how to make the enzyme, and Example 2 and Example 3 describe how to prepare BNCs and scaffolded BNCs comprising Bbh1 and use the scaffolded BNCs to synthesize glycans.

EM-2-3A (EM-2-3-1-1-1) is depicted in FIG. 1E. The glycan synthesis enzyme is Te2FT (α1,2-fucosyltransferase from Thermosynechococcus elongatus; for enzyme expression see Example 4) that transfers L-fucose-GDP to a Gal acceptor to form an α-1,2-fucosylated product while expelling GDP. The module is termed an extension elementary module that functionalizes a glycan, including but not limited to, HMO core structures with L-fucose. In certain embodiments, a BNC of this invention comprises Te2FT.

EM-2-3B (EM-2-3-1-1-2) is depicted in FIG. 1F. The glycan synthesis enzyme is WbgL (α1,2-fucosyltransferase from Escherichia coli; enzyme is expressed) that transfers L-fucose-GDP to a Gal acceptor to form an α-1,2-fucosylated product while expelling GDP. See Example 4. The module is termed an extension elementary module that functionalizes glycans, including but not limited to, HMO core structures with L-fucose. In certain embodiments, a BNC of this invention comprises WbgL.

EM-2-3C (EM-2-3-1-2-2) is depicted in FIG. 1G. The glycan synthesis enzyme is Bf13FT (α1,3,4-fucosyltransferase from Bacteroides fragilis; enzyme is expressed) that transfers L-fucose-GDP and a GlcNAc or Glc acceptor to form an α-1,3-fucosylated or α-1,4-fucosylated product while expelling GDP. See Example 4. The module is termed an extension elementary module that functionalizes glycans, including but not limited to, HMO core structures with L-fucose. In certain embodiments, a BNC of this invention comprises Bf13FT.

EM-2-3D (EM-2-3-2-1-1) is depicted in FIG. 1H. The glycan synthesis enzymes is PmST1 (α-2,3-sialyltransferase from Pasteurella multocida wild or its mutants; for enzyme expression see Example 4) that transfers Neu5Ac from Neu5Ac-CMP produced from module EM1.4A to Gal with an α-2,3 linkage while expelling CMP. The module is termed an extension elementary module that functionalizes glycans, including but not limited to, HMO core structures with Neu5Ac. In certain embodiments, a BNC of this invention comprises PmST1.

EM2-3-E (EM-2-3-2-2-2) is depicted in FIG. 1I. The glycan synthesis enzyme is ST6GAL1 (α-2,6-sialyltransferase from Homo sapiens; enzyme is expressed) that transfers Neu5Ac from Neu5Ac-CMP to Gal with an α-2,6 linkage while expelling CMP. The module is termed an extension elementary module that functionalizes glycans, including but not limited to, HMO core structures with Neu5Ac. In certain embodiments, a BNC of this invention comprises ST6GAL1.

EM-2-3F (EM-2-3-2-2-8) is depicted in FIG. 1J. The glycan synthesis enzyme is STGALNAC5 (α-2,6-sialyltransferase from Homo sapiens; supplied by Glycoexpression Technologies) that transfers Neu5Ac from Neu5Ac-CMP to GlcNAc with an α-2,6 linkage while expelling CMP. The module is termed an extension elementary module that functionalizes glycans, including but not limited to, HMO core structures with Neu5Ac. In certain embodiments, a BNC of this invention comprises STGALNAC5.

EM-2-3G (EM-2-3-5-1-1) is depicted in FIG. 1K. The glycan synthesis enzyme is Cvβ3Ga1T (β-3-galactosyltransferase from Chromobacterium violaceum;

enzyme is expressed) that transfers Gal to a GlcNAc residue while expelling UDP. See Example 4. The module is termed an extension elementary module that elongates glycans, including but not limited to, HMO core structures to make type I structures. In certain embodiments, a BNC of this invention comprises Cvβ3Ga1T.

EM-2-3H (EM-2-3-5-2-1) is depicted in FIG. 1L The glycan synthesis enzyme is NmLgtB (β-4-galactosyltransferase from Neisseria meningitidis; enzyme is expressed) that transfers Gal to GlcNAc while expelling UDP. See Example 4. The module is termed an extension elementary module that elongates glycans, including but not limited to, a HMO core structures to make type II structures. In certain embodiments, a BNC of this invention comprises NmLgtB.

EM-3-1A (EM-3-1-2-2) is depicted in FIG. 1M. They glycan synthesis enzyme is NDPK (Nucleoside 5′-Diphosphate Kinase from E. coli; for enzyme expression see Example 4) which recycles the GDP produced by fucosyltransferases to GTP with equimolar amounts of ATP that is converted to ADP. The module is termed a regeneration elementary module that regenerates GTP from GDP. In certain embodiments, a BNC of this invention comprises NDPK.

Module EM-3-1C (EM-3-1-3-2) is depicted in FIG. 1N. The glycan synthesis enzymes CMPK (Cytidine 5′-Monophosphate Kinase from E. coli; for enzyme expression see Example 4) and NDPK (Nucleoside 5′-Diphosphate Kinase from E. coli; for enzyme expression see Example 4) which convert CMP to CTP with the consumption of two molar equivalents of ATP. The module is termed a regeneration elementary module. In certain embodiments, a BNC of this invention comprises CMPK.

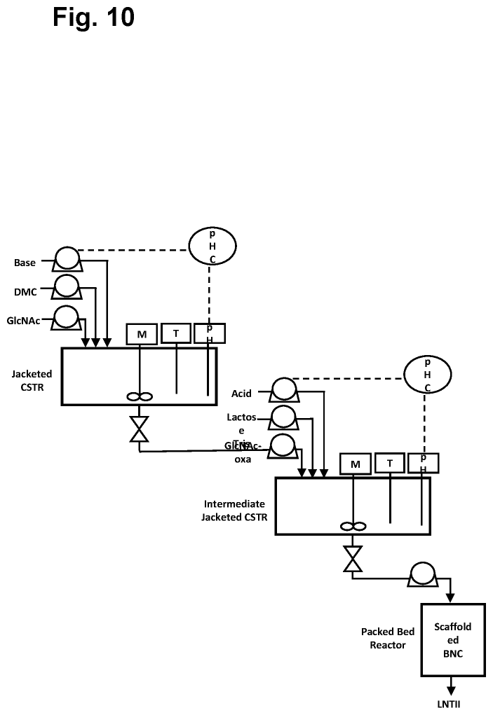

EM-3-2A (EM-3-2-1-1-1) is depicted in FIG. 10. The glycan synthesis enzyme is SuS (AtSuS1, sucrose synthase from Arabidopsis thaliana; enzyme) which converts UDP and sucrose to Glc-UDP and fructose. The module is termed a regeneration elementary module that regenerates Glc-UDP from UDP. In certain embodiments, a BNC of this invention comprises SuS.

EM-5-1A (EM-5-1-1) is depicted in FIG. 1P. The glycan synthesis enzyme is PmPpa (Inorganic pyrophosphorylase, from Pasteurella multocida; for enzyme production see Example 4) which converts pyrophosphate into two equivalents of inorganic phosphate. The module is termed a support elementary module that shifts the equilibria of sugar activation steps. In certain embodiments, a BNC of this invention comprises PmPpa.

EM-6-3A (EM-6-3-1) is depicted in FIG. 1Q. The glycan synthesis enzyme is exo-α-sialidase (exo-α-sialidase, from Salmonella typhimurium) which removes a terminal Neu5Ac residue from glycans, including but not limited to, HMO structures. The module is termed a sugar-removal elementary module that removes a Neu5Ac residue. In certain embodiments, a BNC of this invention comprises exo-α-sialidase.

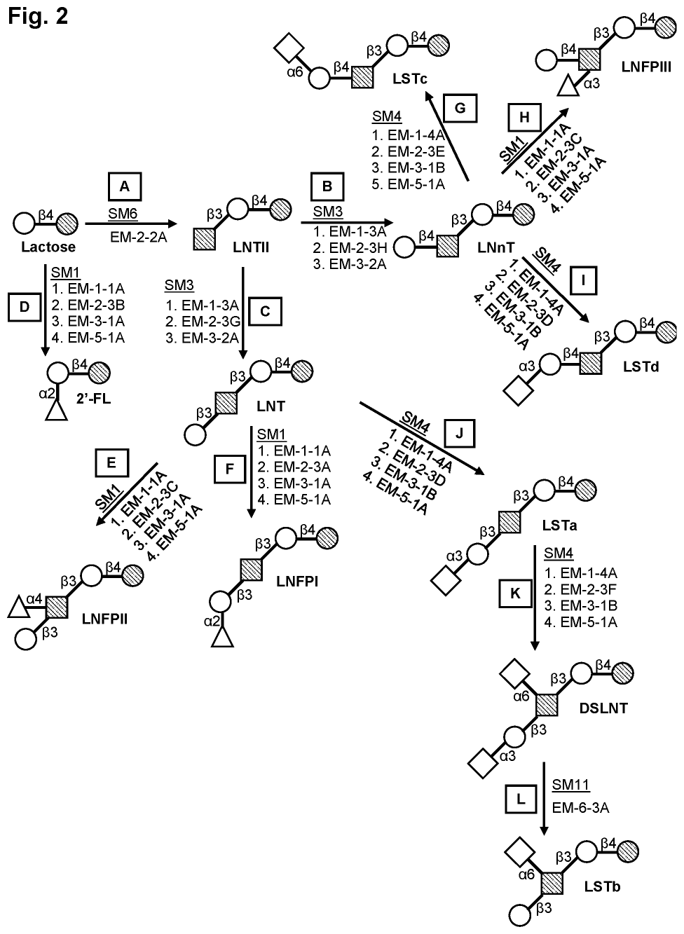

FIGS. 2-8 and FIG. 10 depict the use of certain elementary modules and system modules in methods to prepare glycans. In certain embodiments, the EM is EM1.1A, EM1.4A, EM2.2A, EM2.3A, EM2.3B, EM2.3D, EM3.1A, EM3.1B, or EM5.1A. In another embodiment, the EM is EM2.2A. In another embodiment, the EM is EM1.1A, EM2.3B, EM3.1A, and EM5.1A. In another embodiment, the EM is EM1.4A, EM2.3D, EM3.1B, and EM5.1A.

In any embodiment, the glycan synthesis enzyme is PmST1, NmCSS, HmFucT, Te2FT, FKP, PmPpa, NDPK, CMPK, or Bbh1.

Elementary modules and system modules of the invention are defined herein and in Table 1 below. All subcategories including all sublevels of subcategories may be substituted for each subcategory, including in an EM of Table 1. For example, EM-1-1-1 or EM-1-1-1-1 maybe used instead of EM-1-1 in SM-1, and EM-2-3-1-2-10 may be used instead of EM-2-3 in SM-1.

EM-1: Sugar Activation module

EM-2: Sugar Extension module

EM-3: Reagent Regeneration module

EM-4: Sugar Functionalization module

EM-5: Support Enzyme module

EM-6: Sugar Removal module

TABLE 1
Combination of elementary modules
(EM) to form system modules (SM)

| | | | | |
|---|---|---|---|---|
| EM-1+ | EM-2+ | EM-3+ | EM-5 | =SM |
| EM-1-1+ | EM-2-3+ | EM-3-1+ | EM-5-1 | =SM-1 |
| EM-1-2+ | EM-2-3+ | EM-3-2/EM-3-3 | — | =SM-2 |
| EM-1-3+ | EM-2-3+ | EM-3-2 | — | =SM-3 |
| EM-1-4+ | EM-2-3+ | EM-3-1+ | EM-5-1 | =SM-4 |
| EM-1-5+ | EM-2-1 | — | — | =SM-5 |
| — | EM-2-2 | — | — | =SM-6 |
| EM-4+ | | EM-3 | | |
| EM-4-1 | — | — | — | =SM-7 |
| EM-4-2+ | — | EM-3-3 | — | =SM-8 |
| EM-6 | | | | |
| EM-6-1 | — | — | — | =SM-9 |
| EM-6-2 | — | — | — | =SM-10 |
| EM-6-3 | — | — | — | =SM-11 |

The invention provides the following general reaction schemes.

SM-1

EM-1.1: Sugarₐ [symbol] ATP Sugarₐ-1P+ADP Sugarₐ-1-P+NTP [symbol] Sugarₐ-NDP+PPᵢ

EM-2.3: Sugarₐ-NDP+Sugar_{b} [symbol] Sugarₐ-Sugar_{b}+NDP

EM-3.1: NDP+X-P [symbol] NTP+X

EM-5.1: PPᵢ [symbol] 2Pᵢ

SM-2

EM-1.2: UDP-Glc+2NAD+H₂O [symbol] UDP-GlcA+2NADH

EM-2.3: UDP-GlcA+Sugar [symbol] GlcA-Sugar+UDP

EM-3.2: UDP+sucrose [symbol] UDP-Glc+fructose

EM-3.3: NADH+ [symbol] pyruvate NAD⁺+L-lactate

SM-3

EM-1.3: UDP-Glc [symbol] UDP-Gal

EM-2.3: UDP-Gal+Sugar [symbol] Gal-Sugar+UDP

EM-3.2: UDP+sucrose [symbol] UDP-Glc+fructose

SM-4

EM-1.4: Sugarₐ+CTP [symbol] CMP-Sugarₐ+PPi

EM-2.3: CMP-Sugarₐ+Sugar_{b} [symbol] Sugarₐ-Sugar_{b}+CMP

EM-3.1: CMP+2X-P [symbol] CTP+2X

EM-5.1: [symbol] PPᵢ2Pᵢ

SM-5

EM-1.5: Sugarₐ+ATP [symbol] Sugarₐ-1P+ADP

EM-2.3: Sugarₐ-1P+Sugar_{b} [symbol] Sugarₐ-Sugar_{b}+Pi

SM-6

EM-2.2: Sugarₐ-X+Sugar_{b} [symbol] Sugarₐ-Sugar_{b}+X

SM-7

EM-4.1: UDP-glcNAc+Mannose-X [symbol] 6P-Mannose-X+UMP+GlcNAc

SM-8

EM-4.2: PAPS+Sugar [symbol] Sugar-O/N-SO3+PAP

EM-3.3: PAP+arylsulfate [symbol] Phenol+PAPS

SM-9

EM-6.1: Fuc-Sugar [symbol] Fucose+Sugar

SM-10

EM-6.1: Gal-Sugar [symbol] Galactose+Sugar

SM-11

EM-6.1: Neu5Ac-Sugar [symbol] Neu5Ac+Sugar

The invention provides the following elementary modules (EM). All enzymes are assumed to be immobilized (in certain aspects of this invention) and of bacterial origin unless otherwise noted.

EM-1. This elementary module serves to activate a sugar in preparation for the transfer of this sugar (sugar donor) to a sugar acceptor.

EM-1-1. This sugar activation subcategory consists of the combination of one kinase and one nucleotide transferase, or a natural or synthetic fusion enzyme that integrates both functions into one enzyme.

EM-1-1-1. A combination of Galactokinase (GalK, EC 2.7.1.6) and Galactose-1-phosphate uridyltransferase (Gal-1-phosphate-UDP T, EC 2.7.7.64) that catalyzes the conversion of Galactose to UDP-Galactose.

EM-1-1-1-1. BiGalK/BiUSP from Bifidobacterium infantis

EM-1-1-2. A combination of N-acetylglucosamine kinase (G1cNAcK, EC 2.7.1.162) and N-Acetylglucosamine-1-phosphate-uridyltransferase (GlmU, EC 2.7.7.23) that catalyzes the conversion of GlcNAc to UDP-GlcNAc.

EM-1-1-2-1. BiNahK from Bifidobacterium infantis, HmG1mU from Helicobacter mustelae.

EM-1-1-2-2. BiNahK from Bifidobacterium infantis, and CjG1mU from Campylobacter jejuni.

EM-1-1-3. A combination of Fucokinase (fucK, EC 2.7.1.52) and Fucose-1-phosphate-guanylyltransferase (Fuc-1P-GDP T, EC 2.7.7.30) that catalyzes the conversion of L-fucose to GDP-fucose.

EM-1-1-4. A combination of Glucuronokinase (GlcA K, EC 2.7.1.43) and Glucurono-1-phosphate-uridyltransferase (GlcA-1-phosphate-UDP T, EC 2.7.7.44) that catalyzes the conversion of GlcA to UDP-GlcA.

EM-1-1-5. A combination of Glucokinase (Glc K, EC 2.7.1.2) and Glucose-1-phosphate-uridyltransferase (Glc-1-phosphate-UDP T, EC 2.7.7.9) that catalyzes the conversion of Glc to UDP-Glc.

EM-1-1-6. A combination of Mannokinase (Man K, EC 2.7.1.7) and Mannose-1-phosphate-guanyltransferase (Man-1-phosphate-GDP T, EC 2.7.7.13, 2.7.7.22) that catalyzes the conversion of Man to GDP-Man.

EM-1-1-7. A combination of Rhamnokinase (Rha K, EC 2.7.1.5) and Rhamnose-1-phosphate-uridyltransferase (Rha-1-phosphate-UDP T) that catalyzes the conversion of Rha to UDP-Rha.

EM-1-1-8. A natural fusion enzyme (Bifunctional fucokinase/L-fucose-1-P-guanylyltransferase=FKP, EC 2.7.1.52/2.7.7.30) that catalyzes the conversion of L-fucose to GDP-fucose.

EM-1-1-8-1. BfFKP from Bacteroides fragilis

EM-1-1-9. A synthetic fusion enzyme combining N-acetylglucosamine kinase (G1cNAcK, EC 2.7.1.162) and N-Acetylglucosamine-1-phosphate-uridyltransferase (GlmU, EC 2.7.7.23) that catalyzes the conversion of GlcNAc to UDP-GlcNAc.

EM-1-1-9-1. BINahK-EcGlmU from Bifidobacterium infantis and Escherichia coli.

EM-1-2. This sugar activation subcategory has the capacity to produce activated sugars via oxidation from a structurally related activated sugar.

EM-1-2-1. An oxidase (UDP-Glc-6-dehydrogenase, EC 1.1.1.22) that catalyzes the conversion of UDP-Glc to UDP-GlcA (UDP-Glucuronic acid).

EM-1-3. This sugar activation subcategory has the capacity to produced activated sugars via isomerization from a structurally related activated sugar.

EM-1-3-1. An epimerase (UDP-Gal-4-epimerase, EC 5.1.3.2) that catalyzes the conversion of UDP-Glc to UDP-Gal.

EM-1-3-1-1. EcGalE from Escherichia colt.

EM-1-3-1-2. StGalE from Streptococcus thermophilus.

EM-1-4. This sugar activation subcategory of GlycoSynthetases allows to produce activated sugars from the respective un-activated sugar and cytidine triphosphate.

EM-1-4-1. A GlycoSynthetase (CMP-sialic acid synthetase=CSS, EC 2.7.7.43) that catalyzes the conversion of neuraminic acid (Neu5Ac) and cytidine triphosphate (CTP) to CMP-Neu5Ac.

EM-1-4-1-1. from Neisseria meningitides.

EM-1-4-2. A GlycoSynthetase (3-deoxy-manno-octulosonate cytidyl transferase synthetase=CMP-KDO synthetase=CKS, EC 2.7.7.38) that catalyzes the conversion of 3-deoxy-manno-octulosonate (KDO) and cytidine triphosphate (CTP) to CMP-KDO.

EM-1-5. This sugar activation subcategory of kinases allows to produce phosphorylated (1-P) monosaccharides.

EM-1-5-1. A kinase (Galactokinase, EC 2.7.1.6) that catalyzes the conversion of galactose (Gal) to galactose-1-phosphate (Gal-1-P).

EM-1-5-1-1. BiGalK from Bifidobacterium infantis.

EM-1-5-2. A kinase (Glucokinase, EC 2.7.1.2) that catalyzes the conversion of glucose (Glc) to glucose-1-phosphate (Glc-1-P).

EM-2. This elementary module is a sugar extension module that adds an activated sugar to a sugar acceptor.

EM-2-1. This sugar extension subcategory consists of a sugar phosphorylase that adds a phosphorylated (1-P) sugar donor to a sugar acceptor.

EM-2-1-1. A phosphorylase (1,3-β-galactosyl-N-acetylhexosamine phosphorylase=Galacto-N-biose phosphorylase=GalHexNAcP, EC 2.4.1.211) that catalyzes the conversion of Gal-1P (sugar donor) to GlcNAc with a β-1,3 linkage.

EM-2-1-1-1. BiGalHexNAcP from Bifidobacterium infantis

EM-2-2. This sugar extension subcategory consists of a GlycoSynthetase that adds an activated monosaccharide or oligo to a sugar acceptor.

EM-2-2-1. A fucosidase (EC 3.2.1.51) that catalyzes the transfer of activated L-fucose (nitrophenylated L-fucose or fluoronated L-fucose) onto either Galactose, Glucose, or GlcNAc.

EM-2-2-1-1. α-1,2-fucosidase (EC 3.2.1.63).

EM-2-2-1-2. α-1,3-fucosidase (EC 3.2.1.111).

EM-2-2-1-3. α-1,4-fucosidase (EC not known).

EM-2-2-2. A neuraminidase (sialidase, EC 3.2.1.18) that catalyzes the transfer of activated neuraminic acid (4MU-Neu5Ac, colominic acid, fetuin or sialylconjugates) onto either Galactose, GlcNAc or neuraminic acid.

EM-2-2-2-1. α-2,3-neuraminidase.

EM-2-2-2-2. α-2,6-neuraminidase.

EM-2-2-2-3. α-2,8-neuraminidase.

EM-2-2-3. A β-N-acetylhexosaminidase (β-N-acetylglucosaminidase, EC 3.2.1.52) that catalyzes the transfer of an activated GlcNAc (oxazolinated GlcNAc, nitrophenylated GlcNAc or fluorinated GlcNAc) to Galactose, GlcA, Mannose, GalNAc or GlcNAc.

EM-2-2-3-1. β-1,3-N-acetylglucosaminidase.

EM-2-2-3-1-1 Bbh1 from Bifidobacterium bifidum.

EM-2-2-4. A β-N-acetylgalactosaminidase (EC 3.2.1.53) that catalyzes the transfer of an activated GalNAc (oxazolinated GalNAc, nitrophenylated GalNAc or fluorinated GalNAc) to Galactose, GlcA or IdoA.

EM-2-2-4-1. β-1,4-N-acetylgalactosaminidase.

EM-2-2-5. An α or β-galactosidase (EC 3.2.1.22, 3.2.1.23) that catalyzes the transfer of activated Gal (nitrophenylated Gal, fluorinated Gal or lactose) to Galactose, Glucose, GlcNAc or GalNAc.

EM-2-2-5-1. α-1,2-galactosidase.

EM-2-2-5-2. α-1,3-galactosidase.

EM-2-2-5-3. α-1,4-galactosidase.

EM-2-2-5-4. α-1,6-galactosidase.

EM-2-2-5-5. β-1,3-galactosidase.

EM-2-2-5-6. β-1,4-galactosidase.

EM-2-2-5-7. β-1,6-galactosidase.

EM-2-2-6. A β-glucuronidase (EC 3.2.1.31) that catalyzes the transfer

of an activated glucuronic acid GlcA (nitrophenylated GlcA or fluorinated GlcA) to Galactose, Glucose GalNAc or GlcNAc.

EM-2-2-6-1. β-1,3-glucuronidase.

EM-2-2-7. An α or β-glucosidase (EC 3.2.1.20, 3.2.1.21) that catalyzes the transfer of activated Glucose Glc (nitrophenylated Glc or fluorinated Glc) to Mannose, GlcNAc or Gal.

EM-2-2-7-1. β-1,3-glucosidase.

EM-2-2-7-2. β-1,4-glucosidase.

EM-2-2-7-3. β-1,6-glucosidase.

EM-2-2-7-4. α-1,4-glucosidase.

EM-2-2-8. An α or β-mannosidase (EC 3.2.1.24, 3.2.1.25) that catalyzes the transfer of activated Mannose Man (nitrophenylated Man or fluorinated Man) to Man or GlcNAc.

EM-2-2-8-1. α-1,2-mannosidase.

EM-2-2-8-2. α-1,3-mannosidase.

EM-2-2-8-3. β-1,3-mannosidase.

EM-2-2-8-4. β-1,4-marmosidase.

EM-2-2-9. An β-xylosidase (EC 3.2.1.37) that catalyzes the transfer of

activated Xylose Xyl (nitrophenylated Xyl or fluorinated Xyl) to xylose, mannose, galactose or glcNAc.

EM-2-2-9-1. β-1,4-xylosidase.

EM-2-2-10. An endo specific glycosynthetase that transfers sugar oligosaccharides to acceptor glycans.

EM-2-2-10-1. arabinogalactan endo-β-1,4-galactanase (EC 3.2.1.89).

EM-2-2-10-2. mannosyl-glycoprotein endo-β-N-acetylglucosaminidase (EC 3.2.1.96).

EM-2-2-10-3. endo-α-N-acetylgalactosaminidase (EC 3.2.1.97).

EM-2-2-10-4. blood-group-substance endo-1,4-β-galactosidase (EC 3.2.1.102).

EM-2-2-10-5. keratan-sulfate endo-1,4-β-galactosidase (EC 3.2.1.103).

EM-2-2-10-6. glycoprotein endo-α-1,2-mannosidase (EC 3.2.1.130).

EM-2-2-10-7. lacto-N-biosidase (EC 3.2.1.140).

EM-2-2-10-7-1 LnbB from Bifidobacterium bifidum.

EM-2-2-10-8. mannosylglycoprotein endo-β-mannosidase (EC 3.2.1.152).

EM-2-3. This sugar extension subcategory consists of a Glycotransferase that adds an activated monosaccharide to a sugar acceptor.

EM-2-3-1. A fucosyltransferase that catalyzes the transfer of GDP-fucose (GDP-Fuc) onto either Galactose, Glucose or GlcNAc.

EM-2-3-1-1. α-1,2-fucosyltransferase (EC 2.4.1.69).

EM-2-3-1-1-1 Te2FT from Thermosynechococcus elongatus.

EM-2-3-1-1-2 WbgL from Escherichia coli.

EM-2-3-1-1-3 HmFucT from Helicobacter mustelae.

EM-2-3-1-1-4 FUT1 from Homo sapiens.

EM-2-3-1-1-5 FUT2 from Homo sapiens.

EM-2-3-1-2. α-1,3-fucosyltransferase (EC 2.4.1.152, EC 2.4.1.214).

EM-2-3-1-2-1 HpFucT from Helicobacter pylori.

EM-2-3-1-2-2 Bf1,3FT from Bacteroides fragilis.

EM-2-3-1-2-3 Hp3/4FT from Helicobacter pylori.

EM-2-3-1-2-4 FUT3 from Homo sapiens.

EM-2-3-1-2-5 FUT4 from Homo sapiens.

EM-2-3-1-2-6 FUT5 from Homo sapiens.

EM-2-3-1-2-7 FUT6 from Homo sapiens.

EM-2-3-1-2-8 FUT7 from Homo sapiens.

EM-2-3-1-2-9 FUT9 from Homo sapiens.

EM-2-3-1-2-10 FUT11 from Homo sapiens.

EM-2-3-1-3. α-1,4-fucosyltransferase (EC 2.4.1.65).

EM-2-3-1-3-1 Hp3/4FT from Helicobacter pylori.

EM-2-3-1-3-2 FUT2 from Homo sapiens.

EM-2-3-1-4. α-1,6-fucosyltransferase (EC 2.4.1.68).

EM-2-3-1-4-1 FUT8 from Homo sapiens.

EM-2-3-2. A sialyltransferase that catalyzes the transfer of CMP-neuraminic acid (CMP-Neu5Ac) onto either GlcNAc, Galactose or NeuSAc.

EM-2-3-2-1. α-2,3-sialyltransferase (EC 2.4.99.4, EC 2.4.99.6, EC 2.4.99.7, EC 2.4.99.9).

EM-2-3-2-1-1. PmST1 (wild type and M144D mutant) from Pasteurella multocida.

EM-2-3-2-1-2. NmST1-NmCSS fusion from Neisseria meningitidis.

EM-2-3-2-1-3. ST3GAL1 from Homo sapiens.

EM-2-3-2-1-4. ST3GAL2 from Homo sapiens.

EM-2-3-2-1-5. ST3GAL3 from Homo sapiens.

EM-2-3-2-1-6. ST3GAL4 from Homo sapiens.

EM-2-3-2-1-7. ST3GAL5 from Homo sapiens.

EM-2-3-2-1-8. ST3GAL6 from Homo sapiens.

EM-2-3-2-2. α-2,6-sialyltransferase (EC 2.4.99.1, EC 2.4.99.3).

EM-2-3-2-2-1. Pd26ST from Photobacterium damsel.

EM-2-3-2-2-2. ST6GAL1 from Homo sapiens.

EM-2-3-2-2-3. ST6GAL2 from Homo sapiens.

EM-2-3-2-2-4. ST6GALNAC1 from Homo sapiens.

EM-2-3-2-2-5. ST6GALNAC2 from Homo sapiens.

EM-2-3-2-2-6. ST6GALNAC3 from Homo sapiens.

EM-2-3-2-2-7. ST6GALNAC4 from Homo sapiens.

EM-2-3-2-2-8. ST6GALNAC5 from Homo sapiens.

EM-2-3-2-2-9. ST6GALNAC6 from Homo sapiens.

EM-2-3-2-3. α-2,8-sialyltransferase (EC 2.4.99.8).

EM-2-3-2-3-1. α-2,3/8-sialyltransferase from Campylobacter jejuni.

EM-2-3-2-3-2. ST8SIA1 from Homo sapiens.

EM-2-3-2-3-3. ST8SIA2 from Homo sapiens.

EM-2-3-2-3-4. ST8SIA3 from Homo sapiens.

EM-2-3-2-3-5. ST8SIA4 from Homo sapiens.

EM-2-3-2-3-6. ST8SIA5 from Homo sapiens.

EM-2-3-3. An N-acetylglucosaminyltransferase that catalyzes the transfer of UDP-N-acetylglucosamine (UDP-GlcNAc) to Galactose, Mannose and GlcNAc.

EM-2-3-3-1. β-1,3-N-acetylglucosaminyltransferase (EC 2.4.1.79, EC 2.4.1.149, EC 2.4.1.222).

EM-2-3-3-1-1. HpLgtA form Helicobacter pylori.

EM-2-3-3-1-2. NmLgtA form Neisseria meningitidis.

EM-2-3-3-1-3. HP1105 from Helicobacter pylori.

EM-2-3-3-1-4. B3GNT2 from Homo sapiens.

EM-2-3-3-1-5. B3GNT3 from Homo sapiens.

EM-2-3-3-1-6. B3GNT4 from Homo sapiens.

EM-2-3-3-1-7. B3GNT7 from Homo sapiens.

EM-2-3-3-1-8. B3GNT8 from Homo sapiens.

EM-2-3-3-1-9. B3GNT9 from Homo sapiens.

EM-2-3-3-2. α-1,4-N-acetylglucosaminyltransferase (EC 2.4.1.223, EC 2.4.1.224).

EM-2-3-3-3. β-1,2-N-acetylglucosaminyltransferase (EC 2.4.1.101, EC 2.4.1.143).

EM-2-3-3-3-1. MGAT1 (G1cNAcT-I) from Homo sapiens.

EM-2-3-3-3-2. MGAT2 (G1cNAcT-II) from Homo sapiens.

EM-2-3-3-4. β-1,4-N-acetylglucosaminyltransferase (EC 2.4.1.144, EC 2.4.1.212).

EM-2-3-3-4-1. MGAT3 (G1cNAcT-III) from Homo sapiens.

EM-2-3-3-4-2. MGAT4A (G1cNAcT-IV) from Homo sapiens.

EM-2-3-3-4-3. MGAT4B (G1cNAcT-IV) from Homo sapiens.

EM-2-3-3-4-4. MGAT4C (G1cNAcT-IV) from Homo sapiens.

EM-2-3-3-5. β-1,6-N-acetylglucosaminyltransferase (EC 2.4.1.102, EC 2.4.1.150, EC 2.4.1.155).

EM-2-3-3-5-1. GCNT2A from Homo sapiens.

EM-2-3-3-5-2. GCNT2B from Homo sapiens.

EM-2-3-3-5-3. GCNT2C from Homo sapiens.

EM-2-3-3-5-4. GCNT3 from Homo sapiens.

EM-2-3-3-5-5. GCNT4 from Homo sapiens.

EM-2-3-3-5-6. MGAT5 (G1cNACT-V) from Homo sapiens.

EM-2-3-4. An N-acetylgalactosyltransferase that catalyzes the transfer of an activated UDP-N-acetylgalactosamine (UDP-GalNAc) to Galactose.

EM-2-3-4-1. α-1,3-N-acetylgalactosyltransferase (EC 2.4.1.40).

EM-2-3-4-1-1. ABO from Homo sapiens.

EM-2-3-4-1-2. BgtA from Helicobacter mustelae.

EM-2-3-4-2. β-1,4-N-acetylgalactosyltransferase (EC 2.4.1.174, EC 2.4.1.175).

EM-2-3-4-2-1. B4GALNT3 from Homo sapiens.

EM-2-3-4-2-2. B4GALNT4 from Homo sapiens.

EM-2-3-5. A galactosyltransferase that catalyzes the transfer of activated UDP-Galactose (UDP-Gal) to Glucose and GlcNAc.

EM-2-3-5-1. β-1,3-galactosyltransferase (EC 2.4.1.122, EC 2.4.1.134).

EM-2-3-5-1-1. CvβGalT from Chromobacterium violaceum.

EM-2-3-5-1-2. WbgO from Escherichia coli.

EM-2-3-5-1-3. CgtB from Campylobacter jejuni.

EM-2-3-5-1-4. B3GALT1 from Homo sapiens.

EM-2-3-5-1-5. B3GALT2 from Homo sapiens.

EM-2-3-5-1-6. B3GALT4 from Homo sapiens.

EM-2-3-5-1-7. B3GALT5 from Homo sapiens.

EM-2-3-5-2. β-1,4-galactosyltransferase (EC 2.4.1.22, EC 2.4.1.38, EC 2.4.1.90, EC 2.4.1.133).

EM-2-3-5-2-1. NmLgtB from Neisseria meningitidis.

EM-2-3-5-2-2. NmLgtB-StGalE from Neisseria meningitidis and Streptococcus thermophilus.

EM-2-3-5-2-3. HpLgtB from Helicobacter pylori.

EM-2-3-5-2-4. B4GALT1 from Homo sapiens.

EM-2-3-5-2-5. B4GALT2 from Homo sapiens.

EM-2-3-5-2-6. B4GALT3 from Homo sapiens.

EM-2-3-5-2-7. B4GALT4 from Homo sapiens.

EM-2-3-5-2-8. B4GALT5 from Homo sapiens.

EM-2-3-5-2-9. B4GALT6 from Homo sapiens.

EM-2-3-5-2-10. B4GALT7 from Homo sapiens.

EM-2-3-5-3. α-1,3-galactosyltransferase (EC 2.4.1.37).

EM-2-3-5-3-1. GTB (human proteins) synthetic gene expressed in E. Coli.

EM-2-3-5-3-2. WbnL from E. Coli.

EM-2-3-5-4. α-1,6-galactosyltransferase (EC 2.4.1.241).

EM-2-3-6. A glucuronic acid transferase that catalyzes the transfer of an activated UDP-glucuronic acid (UDP-GlcA) to Galactose and GlcNAc.

EM-2-3-6-1. β-1,3-glucuronic acid transferase (EC 2.4.1.135, EC 2.4.1.212, EC 2.4.1.226).

EM-2-3-6-2. β-1,4-glucuronic acid transferase (EC 2.4.1.226).

EM-2-3-6-2-1. B4GAT1 from Homo sapiens.

EM-2-3-7. A glucosyltransferase that catalyzes the transfer of activated UDP-Glucose (UDP-Glc) to GlcNAc, Mannose, Glucose or Gal.

EM-2-3-7-1. β-1,2-glucosyltransferase (EC 2.4.1.208).

EM-2-3-7-2. β-1,3-glucosyltransferase (EC 2.4.1.305).

EM-2-3-7-3. α-1,3-glucosyltransferase (EC 2.4.1.256, EC 2.4.1.265, EC 2.4.1.267).

EM-2-3-7-4. α-1,4-glucosyltransferase (EC 2.4.1.374).

EM-2-3-8. A mannosyltransferase that catalyzes the transfer of activated UDP-Mannose (UDP-Man) GlcNAc or Man.

EM-2-3-8-1. α-1,2-mannosyltransferase (EC 2.4.1.131, EC 2.4.1.259, EC 2.4.1.260, EC 2.4.1.270).

EM-2-3-8-2. α-1,3-mannosyltransferase (EC 2.4.1.132, EC 2.4.1.252, EC 2.4.1.258).

EM-2-3-8-3. β-1,4-mannosyltransferase (EC 2.4.1.142, EC 2.4.1.251).

EM-2-3-8-4. α-1,6-mannosyltransferase (EC 2.4.1.257, EC 2.4.1.260).

EM-2-3-9. A rhamnosyltransferase that catalyzes the transfer of activated UDP-Rhamnose (UDP-Rham) to Gal or GlcNAc.

EM-2-3-9-1. α-1,3-rhamnosyltransferase (EC 2.4.1.159, EC 2.4.1.289).

EM-2-3-9-2. α-1,4-rhamnosyltransferase (EC 2.4.1.3751).

EM-2-3-10. A xylosyltransferase that catalyzes the transfer of activated UDP-Xylose (UDP-Xyl) to xylose or GlcNAc.

EM-2-3-10-1. β-1,2-xylosyltransferase (EC 2.4.2.38).

EM-2-3-10-2. α-1,6-xylosyltransferase (EC 2.4.2.39).

EM-3. This elementary module is a reagent regeneration module that recycles spent reagent to its original chemical form.

EM-3-1. This reagent regeneration subcategory consists of nucleotide regeneration.

EM-3-1-1. This nucleotide regeneration subcategory achieves the conversion of UDP to UTP.

EM-3-1-1-1. Uridine-diphosphate kinase (EC 2.7.4.6) converts UDP to UTP coupled to stoichiometric conversion of ATP to ADP.

EM-3-1-1-2 Nucleoside-diphosphate kinase (EC 2.7.4.6) converts UDP to UTP coupled to stoichiometric conversion of ATP to ADP.

EM-3-1-1-2-1. Nucleoside-diphosphate kinase, Saccharomyces cerevisiae.

EM-3-1-1-2-2. Nucleoside-diphosphate kinase, bovine.

EM-3-1-1-3. Pyruvate kinase (EC 2.7.1.40) converts UDP to UTP coupled to stoichiometric conversion of phosphoenolpyruvate (PEP) to pyruvate.

EM-3-1-1-4. Acetate kinase, AcK (EC 2.7.4.1) converts UDP to UTP coupled to stoichiometric conversion of acetyl phosphate (AcPi) to acetate (Ac).

EM-3-1-1-4-1. Acetate kinase, Escherichia coli.

EM-3-1-1-4-2. Acetate kinase, Clostridium acetobutylicum.

EM-3-1-2. This nucleotide regeneration category achieves the conversion of GDP to GTP.

EM-3-1-2-1. Nucleoside-diphosphate kinase (EC 2.7.4.6) converts GDP to GTP coupled to stoichiometric conversion of ATP to ADP.

EM-3-1-2-1-1. Nucleoside-diphosphate kinase, Saccharomyces cerevisiae.

EM-3-1-2-1-2. Nucleoside-diphosphate kinase, bovine.

EM-3-1-2-2. Pyruvate kinase (EC 2.7.1.40) converts GDP to GTP coupled to stoichiometric conversion of phosphoenolpyruvate (PEP) to pyruvate.

EM-3-1-2-3. Acetate kinase, AcK (EC 2.7.4.1) converting GDP to GTP coupled to stoichiometric conversion of acetyl phosphate (AcPi) to acetate (Ac).

EM-3-1-2-3-1. Acetate kinase, Escherichia coli.

EM-3-1-2-3-2. Acetate kinase, Clostridium acetobutylicum.

EM-3-1-3. This nucleotide regeneration category achieves the conversion of CMP to CTP using a one-enzyme or two-enzyme system.

EM-3-1-3-1. Cytidine-monophosphate kinase, CMPK (EC 2.7.4.4) and acetate kinase, AcK (EC 2.7.4.1) convert CMP to CTP coupled to stoichiometric conversion of acetyl phosphate (AcPi) to acetate (Ac).

EM-3-1-3-1-1. Acetate kinase, Escherichia coli.

EM-3-1-3-1-2. Acetate kinase, Clostridium acetobutylicum.

EM-3-1-3-2. Cytidine-monophosphate kinase, CMPK (EC 2.7.4.4) and Nucleoside-diphosphate kinase, NDPK (EC 2.7.4.6) converts CMP to CTP coupled to stoichiometric conversion of two ATPs to two ADPs.

EM-3-1-3-2-1. Nucleoside-diphosphate kinase, Saccharomyces cerevisiae.

EM-3-1-3-2-2. Nucleoside-diphosphate kinase, bovine.

EM-3-1-3-3. Cytidine-monophosphate kinase, CMPK (EC 2.7.4.4) and Cytidine-diphosphate kinase, CDK (EC 2.7.4.6) converts CMP to CTP coupled to stoichiometric conversion of two ATPs to two ADPs.

EM-3-1-3-4. Nucleoside-monophosphate kinase, NMK (EC 2.7.4.4) and Cytidine-diphosphate kinase, CDK (EC 2.7.4.6) converts CMP to CTP coupled to stoichiometric conversion of two ATPs to two ADPs.

EM-3-1-3-5. Nucleoside-monophosphate kinase, NMK (EC 2.7.4.4) and Nucleoside-diphosphate kinase, NDPK (EC 2.7.4.6) converts CMP to CTP coupled to stoichiometric conversion of two ATPs to two ADPs.

EM-3-1-4. This nucleotide regeneration category achieves the conversion of ADP to ATP and may be coupled with the nucleotide regeneration modules requiring stoichiometric ATP including 3-1-1-1, 3-1-1-2, 3-1-2-1, 3-1-3-2, 3-1-3-3, 3-1-3-4, 3-1-3-5.

EM-3-1-4-1. Acetate kinase, AcK (EC 2.7.4.1) converts ADP to ATP coupled to stoichiometric conversion of acetyl phosphate (AcPi) to acetate (Ac).

EM-3-1-4-1-1. Acetate kinase, Escherichia coli.

EM-3-1-4-1-2. Acetate kinase, Clostridium acetobutylicum

EM-3-1-4-2. Pyruvate kinase (EC 2.7.1.40) converts ADP to ATP coupled to stoichiometric conversion of phosphoenolpyruvate (PEP) to pyruvate.

EM-3-1-4-3. Polyphosphate kinase, PpK (EC 2.7.4.1) converts ADP to ATP coupled to stoichiometric conversion of polyphosphate (Pₙ) to acetate Pₙ-1.

EM-3-2. This reagent regeneration subcategory consists of sugar nucleotide regeneration.

EM-3-2-1. This nucleotide regeneration subcategory achieves the conversion of UDP to UDP-Glc.

EM-3-2-1-1. Sucrose Synthase (EC 2.4.1.13) converts UDP and Sucrose to UDP-Glc and the byproduct Fructose.

EM-3-2-1-1-1. Sucrose Synthase (SUS), Arabidopsis

EM-3-3. This cofactor regeneration subcategory achieves the conversion of PAPS from PAP and NADH from NAD in connection with Sulfotransferase (EM-4-2) and UDP-Glc dehydrogenase (EM-1-2) enzyme systems.

EM-3-3-1. Aryl sulfotransferase IV (EC 2.8.2.9) converts PAP (3′-Phosphoadenosine 5′-Phosphate) to PAPS (3′-Phosphoadenosine-5′-phosphosulfate) coupled to stoichiometric conversion of an arylsulfate to the respective phenol.

EM-3-3-2. Oxidases that convert NAD (Nicotinamide adenine dinucleotide) to NADH (1,4-Dihydronicotinamide adenine dinucleotide).

EM-3-3-2-1. Lactate dehydrogenase (EC 1.1.1.27) converts NAD to NADH coupled to the stoichiometric conversion of lactate to pyruvate.

EM-3-3-2-2. Xylose reductase (EC 1.1.1.307) converts NAD to NADH coupled to the stoichiometric conversion of xylitol to D-xylose.

EM-4. This elementary module is a functionalization module that either phosphorylated or sulfates glycans.

EM-4-1. Phosphorylation (O—PO₃) (EC 2.7.8.17).

EM-4-1-1. GNPTG form Homo sapiens.

EM-4-2. Sulfation (O—SO₃).

EM-4-2-1. Carbohydrate sulfotransferase, human enzyme.

EM-4-2-1-1. Carbohydrate sulfotransferase 1 (CHST1, EC 2.8.2.21).

EM-4-2-1-2. Carbohydrate sulfotransferase 2 (CHST2, EC 2.8.2.-).

EM-4-2-1-3. Carbohydrate sulfotransferase 3 (CHST3, EC 2.8.2.17).

EM-4-2-1-4. Carbohydrate sulfotransferase 4 (CHST4, EC 2.8.2.-).

EM-4-2-1-5. Carbohydrate sulfotransferase 5 (CHST5, EC 2.8.2.-).

EM-4-2-1-6. Carbohydrate sulfotransferase 6 (CHST6, EC 2.8.2.-;

keratan sulfotransferase 2.8.2.21).

EM-4-2-1-7. Carbohydrate sulfotransferase 7 (CHST7, EC 2.8.2.17).

EM-4-2-1-8. Carbohydrate sulfotransferase 8 (CHST8, EC 2.8.2.-).

EM-4-2-1-9. Carbohydrate sulfotransferase 9 (CHST9, EC 2.8.2.-).

EM-4-2-1-10. Carbohydrate sulfotransferase 10 (CHST10, EC 2.8.2.-).

EM-4-2-1-11. Carbohydrate sulfotransferase 11 (CHST11, EC 2.8.2.5).

EM-4-2-1-12. Carbohydrate sulfotransferase 12 (CHST12, EC 2.8.2.5).

EM-4-2-1-13. Carbohydrate sulfotransferase 13 (CHST13, EC 2.8.2.5).

EM-4-2-1-14. Carbohydrate sulfotransferase 14 (dermatan 4-sulfotransferase CHST14, EC 2.8.2.35).

EM-4-2-1-15. Carbohydrate sulfotransferase 15 (CHST15, EC 2.8.2.33).

EM-4-2-2. Galactose-3-O-sulfotransferase, human enzyme.

EM-4-2-2-1. Galactose-3-O-sulfotransferase (GAL3ST1, EC 2.8.2.11).

EM-4-2-2-2. Galactose-3-O-sulfotransferase (GAL3ST2, EC 2.8.2.-).

EM-4-2-2-3. Galactose-3-O-sulfotransferase (GAL3ST3, EC 2.8.2.-).

EM-4-2-2-4. Galactose-3-O-sulfotransferase (GAL3ST4, EC 2.8.2.-).

EM-4-2-3. Heparan sulfate O-sulfotransferase, human enzyme.

EM-4-2-3-1. Heparan sulfate 2-O-sulfotransferase 1 (HS2ST1, EC 2.8.2.-).

EM-4-2-3-2. Heparan sulfate 3-O-sulfotransferase 1 (HS3ST1, EC 2.8.2.23).

EM-4-2-3-3. Heparan sulfate 3-O-sulfotransferase 2 (HS3ST2, EC 2.8.2.29).

EM-4-2-3-4. Heparan sulfate 3-O-sulfotransferase 2A1 (HS3ST3A1, EC 2.8.2.30).

EM-4-2-3-5. Heparan sulfate 3-O-sulfotransferase 3B1 (HS3ST3B1, EC 2.8.2.30).

EM-4-2-3-6. Heparan sulfate 3-O-sulfotransferase 4 (HS3ST4, EC 2.8.2.23).

EM-4-2-3-7. Heparan sulfate 3-O-sulfotransferase 5 (HS3ST5, EC 2.8.2.23).

EM-4-2-3-8. Heparan sulfate 3-O-sulfotransferase 6 (HS3ST6, EC 2.8.2.23).

EM-4-2-3-9. Heparan sulfate 6-O-sulfotransferase 1 (HS6ST1, EC 2.8.2.-).

EM-4-2-3-10. Heparan sulfate 6-O-sulfotransferase 2 (HS6ST2, EC 2.8.2.-).

EM-4-2-3-11. Heparan sulfate 6-O-sulfotransferase 3 (HS6ST3, EC 2.8.2.-).

EM-4-2-3-12. (heparan sulfate)-glucosamine 3-sulfotransferase 1 (EC 2.8.2.23).

EM-4-2-3-13. (heparan sulfate)-glucosamine 3-sulfotransferase 2 (EC 2.8.2.29).

EM-4-2-3-14. (heparan sulfate)-glucosamine 3-sulfotransferase 3 (EC 2.8.2.30).

EM-4-2-4. N-deacetylase/N-sulfotransferase, human enzyme (EC 2.8.2.8).

EM-4-2-4-1. N-deacetylase/N-sulfotransferase 1 (NDST1).

EM-4-2-4-2. N-deacetylase/N-sulfotransferase 2 (NDST2).

EM-4-2-4-3. N-deacetylase/N-sulfotransferase 3 (NDST3).

EM-4-2-4-4. N-deacetylase/N-sulfotransferase 4 (NDST4).

EM-4-2-4-5. (heparan sulfate)-glucosamine N-sulfotransferase

EM-5. This elementary module is a Support enzyme module.

EM-5-1. Pyrophosphorylase (EC 3.6.1.1) converts pyrophosphate (PPi) to monophosphate (Pi).

EM-5-1-1 PmPpa from Pasteurella multocida.

EM-6. This elementary module is a sugar removal module that hydrolyzes glycans to gain access to asymmetric glycan structures.

EM-6-1. Fucosidase.

EM-6-1-1. 1,2-α-L-fucosidase (EC 3.2.1.63).

EM-6-1-2. 1,3-α-L-fucosidase (EC 3.2.1.111).

EM-6-1-3. 1,6-α-L-fucosidase (EC 3.2.1.127).

EM-6-1-4. α-L-fucosidase (EC 3.2.1.51).

EM-6-1-4-1. α-Fucosidase from Thermotoga maritima.

EM-6-1-4-2. α-(1-2,3,4,6)-Fucosidase from Homo sapiens.

EM-6-2. Galactosidase.

EM-6-2-1. α-galactosidase (EC 3.2.1.22).

EM-6-2-2. β-galactosidase (EC 3.2.1.23).

EM-6-2-2-1. β-galactosidase from Aspergillus niger.

EM-6-2-2-2. β-galactosidase from Escherichia coli.

EM-6-2-2-3. β-galactosidase from Aspergillus oryzae.

EM-6-2-2-4. β-galactosidase from Kluyveromyces lactis.

EM-6-3. Sialidase.

EM-6-3-1. exo-α-sialidase (EC 3.2.1.18).

EM-6-3-1-1. exo-α-sialidase (Salmonella typhimurium).

EM-6-3-1-2. exo-α-sialidase (Clostridium perfringens).

The invention provides for the ability to embed full systems of enzymes to perform synthesis in one-pot reactions and to combine those with flow manufacturing. Thus, the invention provides for commercially applicable and complex bio-catalysis in flow.

The invention provides a series of highly tunable materials and processes for universal enzyme immobilization based on magnetic metamaterials. The unique enzyme hierarchical immobilization platform provides optimal conditions to immobilize single and full systems of enzymes and allows optimal conditions to be found and adapted for single and full systems of enzymes. It affords enzyme stability, maximal use of substrates (including co-factors) and imparts modularity to flow processes. Accordingly, the invention provides a stabilized enzyme composition comprising a bionanocatalyst and a magnetic scaffold, wherein the bionanocatalyst comprises a glycan synthesis enzyme and magnetic nanoparticles and the magnetic scaffold stabilizes the bionanocatalyst.

One embodiment provides a modular process for producing a glycan, comprising a module that may be a flow cell wherein: said module comprises a magnetic macroporous powder comprising magnetic microparticles, wherein said powder has immobilized a preparation of self-assembled mesoporous aggregates of magnetic nanoparticles containing a glycan synthesis enzyme; wherein a substrate is introduced into said module (or passed through a flow cell) and said substrate is modified to provide a glycan.

Continuous flow reactors including, but not limited to, packed-bed and fixed-bed reactors in tubular format can be combined with upstream and downstream processes that are not continuous making the overall process semi-continuous. For example, the reaction feed for the LNTII reaction (Example 3c-A) may be produced in a continuous stirred tank reactor, the product of which is continuously added to the flow reactor. Microfluidic reactors may also be employed in connection with this invention.

FIG. 10 depicts an embodiment of this invention involving a continuous stirred-tank reactor (CSTR) and illustrating a semi-continuous processing method. Specifically, FIG. 10 depicts the enzymatic reaction scheme for creating LNTII. In the first step, GlcNAc-oxa is produced by pumping GlcNAc, Et₃N and DMC into a jacketed CSTR cooled to 0° C. In the second step, the GlcNAc-oxa is pumped into an intermediate jacketed CSTR cooled to 0° C. along with buffered lactose and acid to maintain pH. This mixture is then pumped into a packed bed reactor containing the enzyme Bbh1 immobilized on the scaffold to produce LNTII. pHC=pH controller; DMC=2-Chloro-1,3-dimethylimidazolinium chloride; GlcNAc=N-acetylglucosamine; CSTR=continuous stirred-tank reactor; M=mixer; T=temperature controller; pH=pH probe.

The invention provides methods to make glycans employing glycan synthesis enzymes. The glycan synthesis enzyme is a natural or a synthetic enzyme, including fusion enzymes.

In one embodiment, the invention provides methods for making glycans by immobilizing an enzyme with magnetic nanoparticles and contacting the immobilized enzyme with appropriate synthetic reagents. The methods may be conducted in batch, flow, semi-continuous, or continuous-flow.

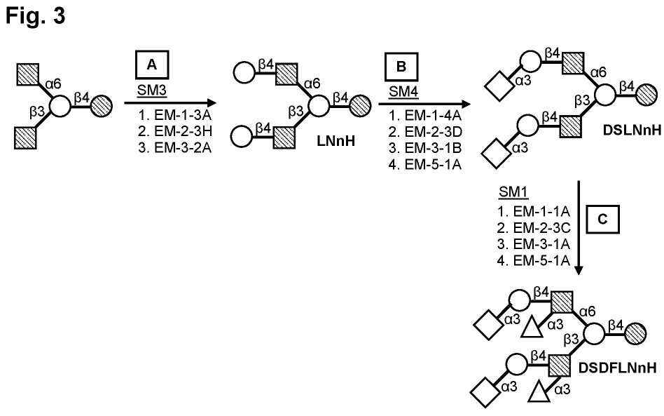

In certain embodiments, this invention provides glycans, methods of synthesizing the glycans, catalysts for use in the glycan synthesis, modules for use in the glycan synthesis, methods for making the catalyst, and methods for making the module. The glycans include, but are not limited to, LNTII, LNT, LNnT, LNFPI, LNFPII, LNFPIII, LSTa, LSTb, LSTc, LSTd, DSLNT, 2′-FL, DSDFLNnH, and 3′SL. FIG. 2 and FIG. 3 depict these embodiments and Examples 1-2 describe the methods.

FIG. 2 depicts elementary modules and their use in the synthesis of LNTII, LNT, LNnT, LNFPI, LNFPII, LNFPIII, LSTa, LSTb, LSTc, LSTd, DSLNT, and 2′-FL. The elementary modules comprise BNCs that are combined into sets of system modules (see FIG. 1).

In one embodiment DSDFLNnH (3′″₃,3′″₆-di-O-α-Sia-(3″₃, 3″₆-di-O-α-Fuc)-LNnH) is produced from 3′″₃,3′″₆-di-O-α-Sia-LNnH, fucose, ATP and GTP with system module 1 (SM1) using four elementary modules (EM1.1A, EM2.3C, EM3.1A, EM5.1A; see FIG. 1 and FIG. 3). BNCs are immobilized on a strontium ferrite scaffold, and the BNC functionalized powder is packed into a column. A syringe pump delivers the reagent stream into module SM1. The reagent stream consists of 25 mM 3′″₃,3′″₆-di-O-α-Sia-LNnH, 250 mM ATP, 60 mM fucose, 40 mM GTP and 10 mM MgCl₂ (see Example 2d-C).

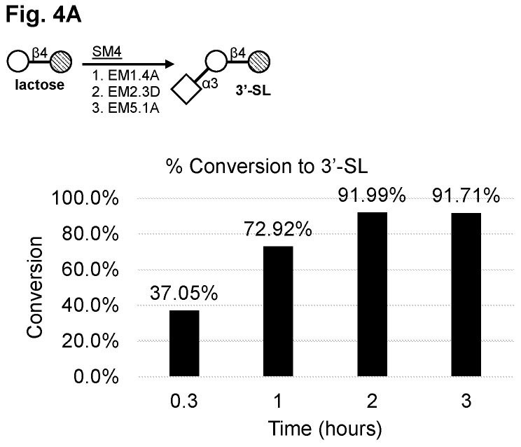

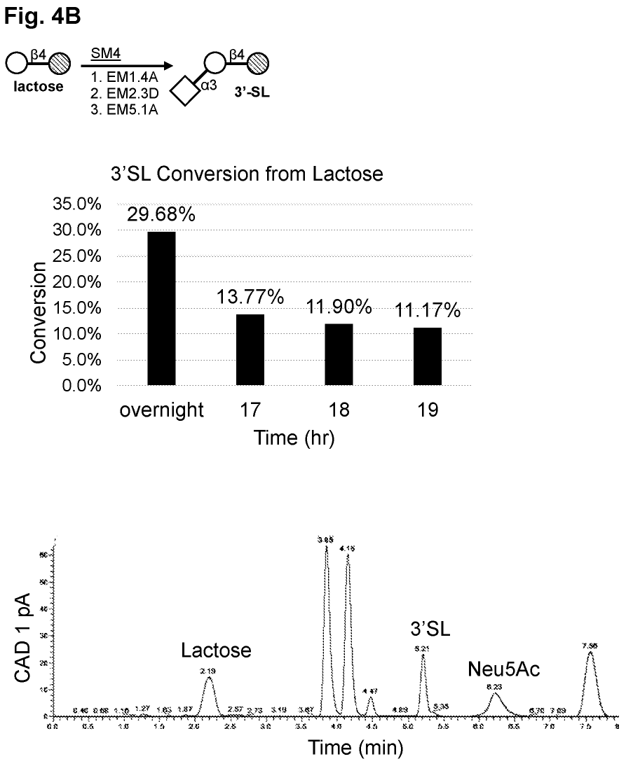

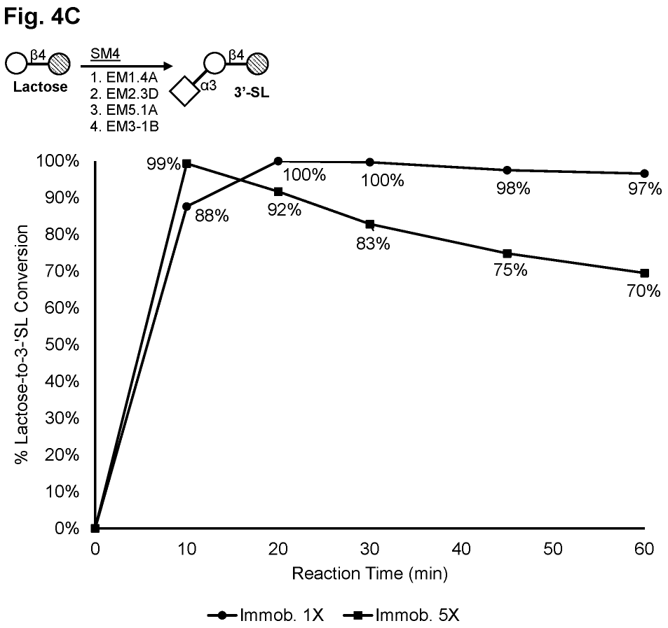

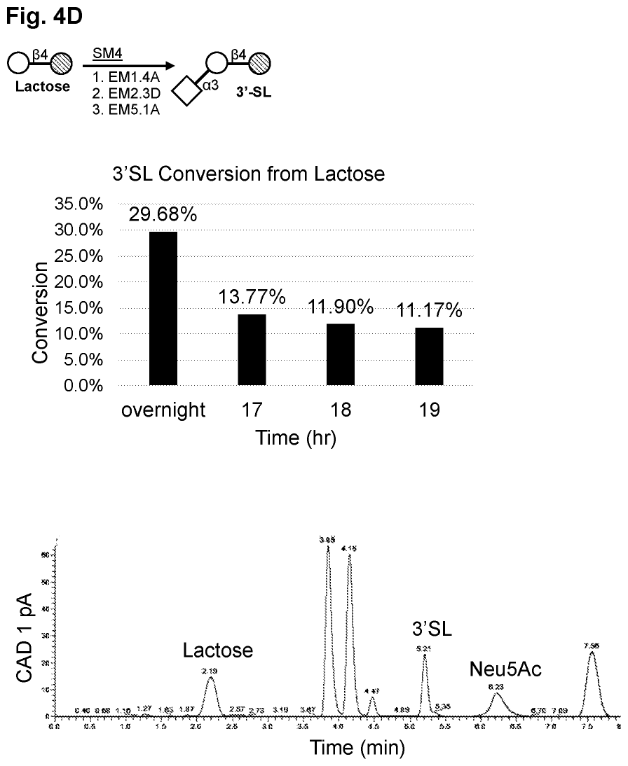

FIG. 4A depicts batch reaction synthesis of 3′SL with scaffolded BNCs and a HPLC analysis of the reaction (see Example 1b). The reaction afforded greater than 75% conversion of 3′-SL from NeuSAc by HPLC analysis (see Example 1b). That yield corresponded to ˜90% of the yield compared to the free enzyme reaction.

FIG. 4B depicts in-flow synthesis of 3′SL with immobilized enzymes, presents a % conversion to 3′-SL as determined by HPLC, and presents a representative HPLC chromatogram (see Example 1c).

Monitoring percent conversion as depicted in FIG. 4C is a method of evaluating methods of the invention and the Scaffolded BNC. Scaffolded BNCs and methods of this invention may be evaluated and monitored using other methods, including but not limited to the Bradford assay that indicates whether the enzymes have been immobilized (Bradford MM. A rapid and sensitive method for the quantitation of microgram quantities of protein utilizing the principle of protein-dye binding. Analytical biochemistry 1976; 72:248-54), and scanning electron microscopy (SEM). FIG. 4C depicts the enzymatic reaction scheme and % conversion of lactose to 3′-SL over time using scaffolded BNCs in a batch reaction. The % conversions were determined by HPLC and are shown for two enzyme loadings (1× and 5×). See Example 3b for details.

In another embodiment, the invention provides 3′SL and the synthesis of 3′-SL in a flow reaction. FIG. 4D depicts the enzymatic reaction scheme and % conversion of lactose to 3′-SL over time using scaffolded BNCs in a flow reaction. Lactose in the presence of SM4 (EM1.4A, EM2.3D, and EM5.1A) N-Acetylneuraminic acid (NeuSAc), and CTP were contacted in a continuous flow reactor (in-flow) with immobilized NmCSS, PmPpa and PmST1 to provide 3′-SL. The % conversions were determined by HPLC and a representative HPLC chromatogram is depicted with the signal measured amperometrically (picoAmps, pA) by charged aerosol detection (CAD). See Example 3c for details.

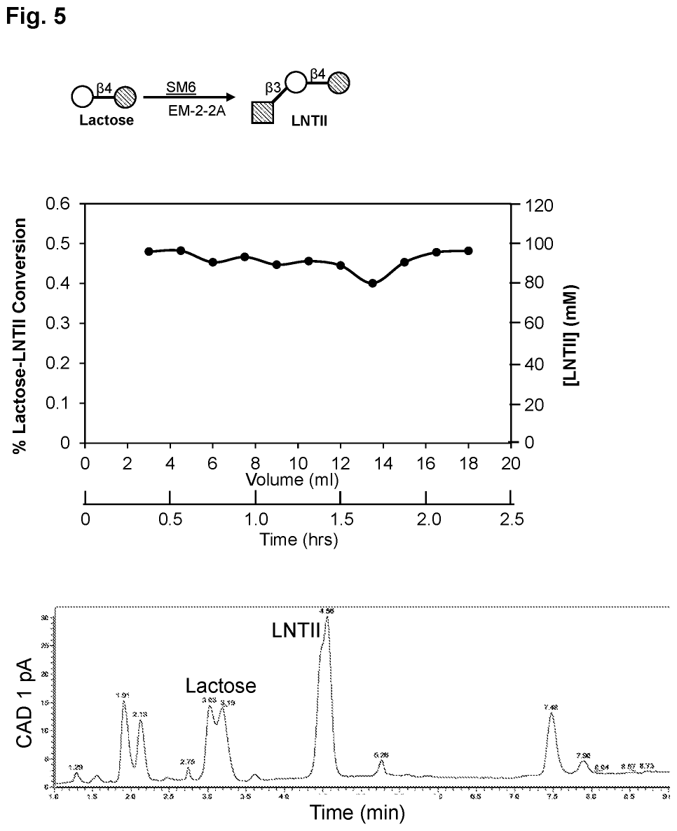

In another embodiment, the invention provides LNTII and the synthesis of LNTII as described in Example 3c-A. in a flow reactor FIG. 5 depicts the enzymatic reaction scheme and % conversion of lactose to LNTII over time. Lactose was reacted with SM6 (EM-2-2A) to provide LNTII. The % conversions were determined by HPLC and a representative HPLC chromatogram is depicted with the signal measured amperometrically (picoAmps, pA) by charged aerosol detection (CAD). See Example 3c-A for details.

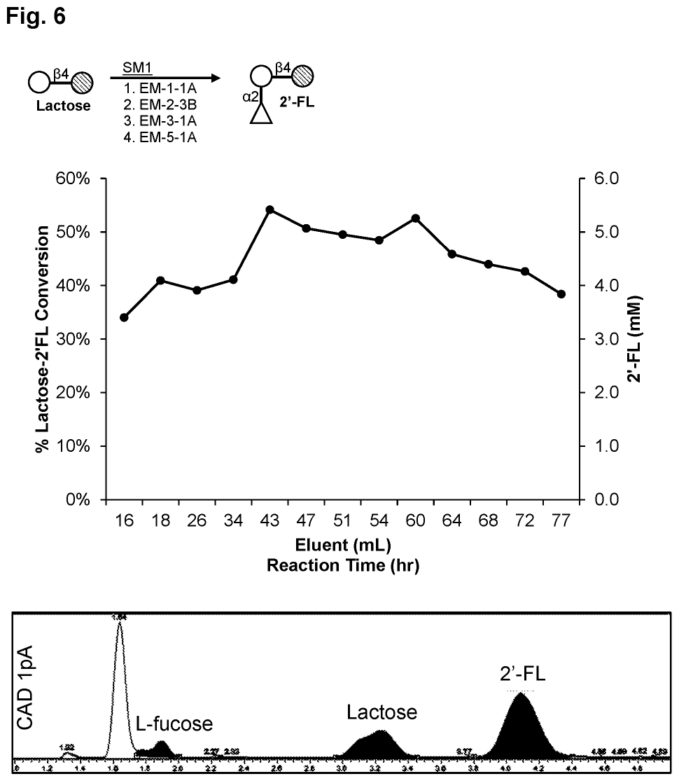

In another embodiment, the invention provides 2′-FL and also provides a model system for Scaffolded BNC methodology. FIG. 6 depicts the enzymatic reaction scheme and % conversion of lactose to 2′-FL over time using NiNTA immobilized enzymes in a flow reactor. The % conversions were determined by HPLC and a representative HPLC chromatogram is depicted with the signal measured amperometrically (picoAmps, pA) by charged aerosol detection (CAD). This method can be used as model system for scale-up and testing other aspects of invention involving Scaffolded BNCs. See Example 3c-B for details.

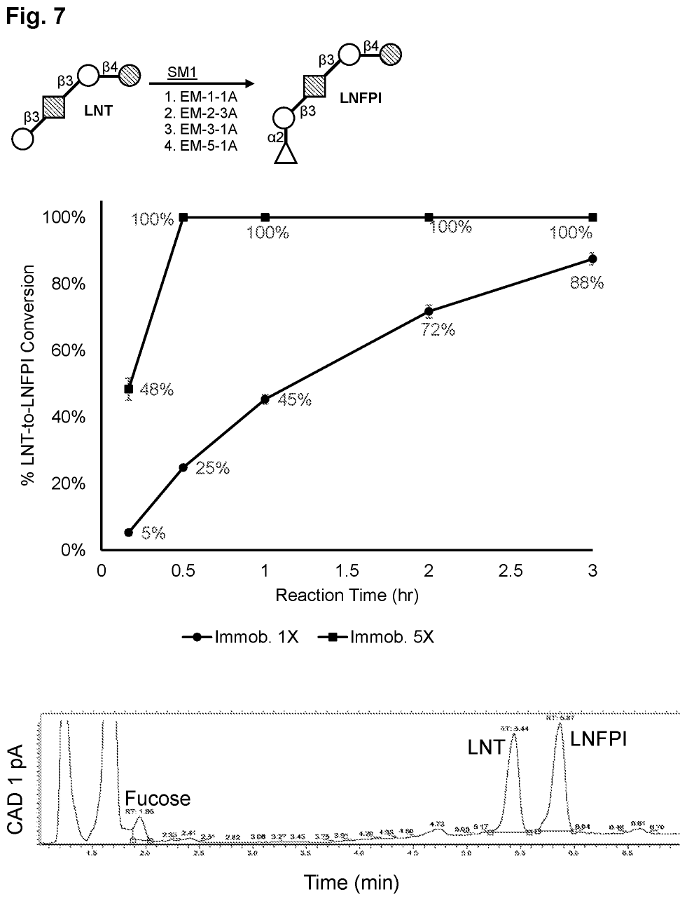

In another embodiment, the invention provides LNFPI a 5-subunit glycan. FIG. 7 depicts the enzymatic reaction scheme and % conversion of LNT to LNFPI over time using scaffolded BNCs in a batch reaction in SM-1 (EM-1-1A, EM-2-3A, EM-3-1A, and EM-5-1A). The % conversions were determined by HPLC and a representative HPLC chromatogram is depicted with the signal measured amperometrically (picoAmps, pA) by charged aerosol detection (CAD). The % conversions are shown for two enzyme loadings (1× and 5×). An 88% conversion was achieved after 180 min with the immobilized 1× enzyme system while a full conversion (100%) was achieved after 30 min with the immobilized 5× enzyme system. See Example 3c-C for details.

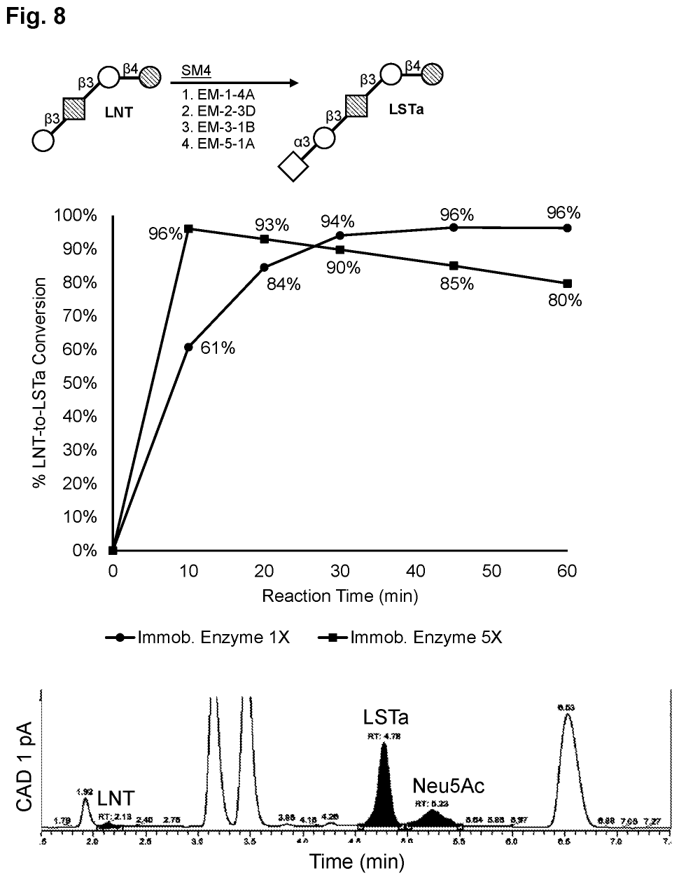

In another embodiment, the invention provides LSTa a 5 subunit glycan. FIG. 8 depicts the enzymatic reaction scheme and % conversion of LNT to LSTa over time using scaffolded BNCs in a batch reaction using SM4 (EM-1-4A, EM-2-3D, EM-3-1B, and EM-5-1A. The % conversions were determined by HPLC and a representative HPLC chromatogram is depicted with the signal measured amperometrically (picoAmps, pA) by charged aerosol detection (CAD). The % conversions are shown for two enzyme loadings (1× and 5×). A 94% conversion was achieved after 30 minutes with the immobilized 1X enzyme system, while a 96% conversion was achieved after 10 min with the immobilized 5X enzyme system See Example 3c-D for details.

Accordingly, the invention provides a method for producing a glycan, comprising using a module (that may be a flow cell) wherein:
a. said module comprises a magnetic macroporous powder comprising magnetic microparticles, wherein said powder has immobilized a preparation of self-assembled mesoporous aggregates of magnetic nanoparticles containing a glycan synthesis enzyme; wherein a substrate is introduced into said module (or passed through a flow cell) and said substrate is modified to provide a glycan.

The invention provides methods comprising 1 or more modules. In a method comprising more than a first module, a first substrate is passed through said first modular flow cell to create a modified substrate; wherein said modified substrate is a second substrate to pass through a second module to create a second modified substrate. The invention provides for sets of modules to be combined allowing the synthesis of complex glycans.

In the method comprising more than a first module, a first substrate is passed through said first modular flow cell to create a modified substrate; wherein said modified substrate is a second substrate to pass through a second module to create a second modified substrate. The invention provides for sets of modules to be combined allowing the synthesis of complex glycans.

Accordingly, the invention provides a method wherein the method comprises a second module (that may be a flow cell) comprising a magnetic macroporous powder comprising magnetic microparticles, wherein said powder has immobilized a preparation of self-assembled mesoporous aggregates of magnetic nanoparticles containing a second glycan synthesis enzyme.

In one embodiment, the method comprises a first module and a second module, a first substrate is passed through said first modular flow cell to create an activated sugar; wherein said activated sugar is a second substrate to pass through said second modular flow cell to create a first sugar multimer product. In this embodiment, the first glycan synthesis enzyme is a sugar activation enzyme and the second glycan synthesis enzyme is a sugar extension (sugar transfer) enzyme.

Compounds (glycans or carbohydrates) that may be prepared according to methods of this invention include, but are not limited to, rare sugars, activated sugars, HMOs, glycans with sugar modifications, glycosylated small molecules, polymerization of fiber sugars, inulins, levans, gluconic acid, invert sugar, flavors and fragrances.

The invention provides a for making a glycan, comprising the steps of preparing a scaffolded bionanocatalyst comprising a glycan synthesis enzyme; contacting the scaffolded bionanocatalyst with a glycan substrate; and converting the glycan substrate into a glycan.

Glycans, are carbohydrate-based compounds featuring one or more monosaccharides linked with a glycosidic bond, including N-linked and O-linked bonds. Activated monosaccharides, oligosaccharides, polysaccharides, plant glycans, animal glycans, and microbe glycans are all within the scope of this invention as are glycoconjugates, such as glycolipid, glycopeptides, glycoproteins, and proteoglycans. Glycans also include humanized glycoproteins, humanized antibodies, and glycoconjugate vaccines. Riley, et al. Nature Reviews Nephrology, vol. 15, pp. 346-366 (2019). Rappuoli, Science Translational Medicine 29 Aug. 2018: Vol. 10, Issue 456.

The glycans may be simple glycans or complex glycan, including linear or branched having any number of sugar units. In certain embodiments, the glycans have five sugar units or more. In certain embodiments, the glycans have 1-10 units. In some embodiments, the glycans have 1-5 units. In certain embodiments, the glycans have 1, 2, 3, 4, 5, 6, 7, 8, 9, 10 units. In certain embodiments, the glycans are straight chained or branched chained. In certain embodiments, the glycans have 1-5 units and are straight chained. In other embodiments, the glycans have 1-5 units and are branched. In other embodiments, the glycans have 1-10 units and are branched.

In certain embodiments, the glycan is an oligosaccharide. In preferred embodiments, the glycan is a human milk oligosaccharides (HMOs).

In certain embodiments, the glycan is 3′SL, LNTII, LNT, LNnT, LNFPI, LNFPII, LNFPIII, LSTa, LSTb, LSTc, LSTd and DSLNT, LNnH, 3′″₃, 3′″₆-di-O-α-Sia-LNnH 3′″₃,3′″₆-di-O-α-Sia-(3″₃,3″₆-di-O-α-Fuc)-LNnH, biantennary sialylated or fucosylated lacto-N-neohexaoses and neoheptaoses, α-2,3-sialyl lacto-N-neopentaose, linear fucosyl- and sialyl-lacto-N-neo-pentaoses, linear lacto-N-neopentaoses, or biantennary lacto-N-neohexaoses and heptaoses. In certain embodiments, the glycans are 3′SL LNTII, LNT, LNnT, LNFPI, LNFPII, LNFPIII, LSTa, LSTb, LSTc, LSTd, DSLNT, 2′-FL, LNnH, DSLNnH, or DSDFLNNH. In other embodiments, the glycans are 3′SL, LNTII, 2′-FL, LNFPI, LSTa.

A method of making a glycan according to this invention may be a batch, flow process, continuous flow, or a semi-continuous flow process. Certain embodiments provide high enzyme loading flow cells or a flow cell and continuous manufacturing. The methods may employ a further step of removing the mesoporous aggregates or materials and replacing them with a fresh preparation of mesoporous aggregates or materials.

Also provided by the invention are devices for producing glycans employing any of the compositions or methods herein. Accordingly, the invention provides a device for producing a glycan, comprising a module or a system module according to any of the modules herein, wherein said module modifies a glycan substrate to produce a glycan. The device optionally comprises a first module and a second module, wherein a first substrate is introduced into said first module to create a modified first substrate; wherein the first modified substrate is a second substrate introduced into the second module to produce a glycan. The device optionally comprises a third module, wherein the glycan is introduced into said third module to create a third modified substrate. The device optionally comprises a fourth module wherein the third modified substrate is introduced into the fourth module to create a fourth modified substrate. The device optionally comprises a fifth module, wherein said fourth modified substrate is introduced into said fifth module to create a fifth modified substrate. The device optionally comprises a sixth module, wherein said fifth modified substrate is introduced into said sixth module to create a sixth modified substrate. Additional modules may be added to prepare additional modified glycans.

A method of making such a device is also provided. One embodiment provides a method of making a device for catalyzing an enzymatic reaction, comprising combining a magnetic macroporous scaffold with self-assembled mesoporous aggregates of magnetic nanoparticles and a glycan synthesis enzyme, wherein said enzyme is magnetically immobilized within or in said mesopores.

Another embodiment provides for catalyzing a reaction between a plurality of substrates, comprising exposing a scaffolded bionanocatalyst according to this invention-to the substrates under conditions in which said scaffolded bionanocatalyst catalyzes said reaction between said substrates. In certain embodiments, the substrates is glycan substrates and the glycan substrates are exposed to a module or a system module of this invention under conditions in which the module catalyzes a reaction between the substrates.

Scaffolds according to the invention are chemically inert, structurally tunable to fit any process, and highly magnetic to ensure full capture of the enzyme-containing cluster.

The process for preparing the magnetic scaffolds is flexible and glycan synthesis in a module provides for convenient, flexible synthesis of glycans.

The process for preparing the magnetic scaffolds is flexible and tunable to manufacture objects using 3D designs that magnetically capture the BNCs. A large surface area may result from the sintering process itself. Materials can also be recycled by removing the BNCs and then re-functionalized them for repeated use. See PCT/US19/53307, incorporated by reference herein in its entirety.

In some embodiments, thermoplastics are Polyethylene (PE) (varying densities, e.g. LDPE, HDPE), Polypropylene (PP), Acrylics: Polyacrylic acids (PAA), Poly(methyl methacrylate) (PMMA), Polyvinyl alcohol (and polyvinyl acetals), Polyamides (Nylon), Polylactic acid (PLA), Polycarbonate (PC), Polyether sulfone (PES), Polystyrene (PS), Polyvinyl chloride (PVC), Acrylonitrile butadiene styrene (ABS), Polybenzimidazole (PBI), Polyoxymethylene (POM), Polyetherether ketone (PEEK), Polyetherimide (PEI), Polyvinylidene fluoride (PVDF), Polytetrafluoroethylene (PTFE/Teflon), Polyacrylonitrile (PAN)) blended with magnetic materials (e.g. magnetite MMP) via melting/extrusion or via coating of the magnetic material by dissolving the plastic in a solvent. In other embodiments, the powders are sintered by a laser using SLS. Porosity may be formed during SLS.

In one embodiment, polypropylene-magnetite materials can be 3D-printed in any shape and form via SLS.

In other embodiments, an extruded composite material is size reduced via cryomilling or another form of milling. In other embodiments, composite powders are sieved to an ideal particle size. In preferred embodiments, the particle sizes are 60+/−20 μm.

Powders or 3D printed objects can be functionalized with BNCs containing

one or more enzymes or enzyme systems. BNCs are magnetically trapped at the surface of the powders or 3D printed objects.

Composite powders may also be optimized for flowability. In some embodiments, 3D objects can be printed to optimize flow within to be used in flow reactors.

In some embodiments, 3D objects and composite powders can be washed from the BNCs by an acid wash, rinsed with water, and then re-functionalized with fresh BNCs.

Highly magnetic scaffolds (Macroporous Magnetic Scaffolds or MMP) are designed to immobilize, stabilize and optimize any BNCs containing enzymes. This includes full enzyme systems at high loading and full activity for the production of small molecules. By combining natural or engineered enzymes, and in some embodiments with cofactor recycling systems, the scaffolds allow one to scale up biocatalysis to innovations to manufacturing scale and production.

MMP made of thermoplastic and magnetic materials of the invention can take the form of magnetic powders that are suitable for flow chemistry application. These powders can be 3D printed by SLS as structures, as functional objects, or as flow cells or plate reactors. High surface areas allow one to maximize the enzyme loading and flow can be engineered within the materials to enable biocatalysis at maximal productivity.

SLS can be used to process nearly any kind of material from metals, ceramics, plastics, and combinations thereof, for tailor-made composite materials. It is critical, however, that the material is available in fine powder form and that the powder particles are operative to fuse when exposing them to heat (Kruth et al., Assembly Automation 23(4):357-371(2003), incorporated by reference herein in its entirety.

When the material lacks those features, or is prone to phase transitions at the temperature range or conditions of the sintering process, the addition of a sacrificial binder can make this process still feasible for that material. Commonly, polymers are used as sacrificial binders in order to expand the range of materials suitable for this technology. After sintering, the sacrificial binder can be either removed by thermal decomposition or kept as part of the composition.

This concept applies to magnetite that loses its permanent magnetic properties above 585° C. This is significantly lower than its melting temperature (1538° C.). Another advantage of using a polymeric matrix to incorporate magnetite particles is that the former can act as a protective barrier to prevent oxidation and corrosion as well as aiding to disperse the magnetite particles. Also, magnetite can mechanically reinforce the polymer. (Shishkovsky et al., Microelectronic Engineering 146:85-91 (2015), incorporated by reference herein in its entirety).

Laser sintering of plastic parts is one of two additive manufacturing processes used for Rapid Manufacturing (Wegner, Physics Procedia 83:1003-1012 (2016), incorporated by reference herein in its entirety). There are several polymer properties that determine its capability to be sintered and produce good quality 3D objects. These include structural properties such crystalline structure (i.e. thermal properties such as Tm, Tg, and Tc), mechanical properties (Young's modulus and elongation at break, etc.), density, particle size, and shape.

In SLS, the temperature-processing window is determined from the difference between the melting and crystallization temperatures of the polymer. For instance, nylon 12 (PA 12) has one of the highest operational windows and is thus a widely used SLS material. In theory, the higher this value is, the easier the material can be sintered. In practice, however there are many more parameters that can still make this process difficult for any specific polymer (Shishkovsky et al., Microelectronic Engineering 146:85-91 (2015)), incorporated by reference herein in its entirety). In order to prevent curling of the sintered part, a low polymer crystallization rate is desired together with a melt index that provides a suitable rheology and surface tension.

Additionally, the bulk density, particle shape, and size distribution of the powder are key factors (Wegner, Physics Procedia 83:1003-1012 (2016), incorporated by reference herein in its entirety). It has been determined that the optimal particle size range is about 40 to about 90 microns. Smaller particles prevent flowability and their rapid vaporization is detrimental to the optical sensors of the sintering device. This can fog the device and lead to inaccurately sintered parts (Goodridge et al. Materials Science 57:229-267 (2012), incorporated by reference herein in its entirety). The powders should have good flowing properties and preferably an approximately round particle shape. This allows good powder spreading during the process. High heat conductivity of the material is desired at the CO₂ laser beam wavelength (10.6 microns). This is not the case for most polymers. The last two requirements can be met by the incorporation of additives such as high-energy absorption materials, e.g. carbon black, to improve heat absorption, and fume silica nanoparticles (talc) to aid the particle flowability with irregularly-shaped particles.

Additive manufacturing (AM), also referred to as 3D printing, involves manufacturing a part by depositing material layer-by-layer. This differs from conventional processes such as subtractive processes (i.e., milling or drilling), formative processes (i.e., casting or forging), and joining processes (i.e., welding or fastening). Quick production time, low prototyping costs, and design flexibility make 3D printing a valuable tool for both prototyping and industrial manufacturing. The three most common types of 3D printers are fused filament fabrication, stereolithography, and selective laser sintering.

Fused filament fabrication (FFF) melts a thermoplastic continuous filament and builds the object layer by layer until the print is complete. Although alternative materials exist, the two most popular filament materials are polylactic acid (PLA) and acrylonitrile butadiene styrene (ABS). FFF printers and materials are among the cheapest on the market but currently have a lower print resolution and build quality.

Stereolithography (SLA) uses a laser to polymerize photosensitive resins. Uncured liquid resin is placed in a vat where a laser is used to cure resin into solid plastic and build the object layer by layer. SLA printers have a much higher resolution than FFF printers due to the fine spot size of the laser and thus can print intricate features and complex shapes. The resins, however, are more expensive than filaments and completed prints currently require post processing with solvents to optimize the surface finish and material characteristics.

Selective laser sintering (SLS) is a powder-based layer-additive manufacturing process generally meant for rapid prototyping and rapid tooling. Laser beams either in continuous or pulse mode are used as a heat source for scanning and joining powders in predetermined sizes and shapes of layers. The geometry of the scanned layers corresponds to the various cross sections of the computer-aided design (CAD) models or stereolithography (STL) files of the object. After the first layer is scanned, a second layer of loose powder is deposited over it, and the process is repeated from bottom to top until the artifact (3D object) is complete.” Kumar, JOM, 55(10), 43-47 (2003), incorporated by reference herein in its entirety.

SLS provides advantages for printing objects with magnetic properties that can be used for immobilizing BNCs. This is because the printing process creates porosity and a high surface area. The surrounding, unsintered powder acts as a natural support that eliminates the need for dedicated support structures. The lack of support structures allows for complex geometries that would otherwise be impossible to manufacture using alternative 3D printing methods. In addition, the nature of sintering itself creates macro and microporous volumes. During the printing process, the laser flashes thermoplastic crystalline thermoplastic powders (e.g. Polypropylene, polystyrene) between their glass transition temperature and melting temperature to generate stiff parts. By avoiding amorphous behavior with a quick laser scan speed (>100 mm/s), powders are sintered in place to form small bonds amongst themselves. The low-density powders trap air in their structures resulting in remarkable porosity and surface area in three dimensions. These pores increase the surface area for enzyme immobilization.

In recent years, industrial use of enzymes has garnered significant attention due to the wide range of potential manufacturing applications. Using enzymes in industrial processes offers several advantages over conventional chemical methods. This includes high catalytic activity, the ability to perform complex reactions, and promoting greener chemistry by reducing by-products and the need for toxic chemicals (Singh et al., Microbial enzymes: industrial progress in 21st century. 3 Biotech. 6(2):174 (2016), incorporated by reference herein in its entirety).

One of the biggest hindrances to widespread biocatalysis use in industrial production is low enzyme stability. This is further hampered by relatively harsh process conditions that can destabilize enzymes and decrease their lifespan (Mohamad et al., Biotechnology, Biotechnological Equipment 29(2):205-220 (2015), incorporated by reference herein in its entirety). Furthermore, the use of free enzymes in these processes are generally lost from the system as waste products and therefore become a costly operating cost. The primary solution to these issues is immobilization of enzymes onto scaffolding to enhance their operational stability and catalytic activity. Enzyme immobilization also provides a method for enzyme recovery, making biocatalytic processes more economically feasible.

Currently, biocatalytic processes for industrial production are generally carried out in batch reactors due to their simplicity and ease of operation. Despite the benefits of using batch reactors, continuous flow systems enable higher productivity and better process control (Wiles C et al., Green Chem. (14):38-54 (2012)). The rapid development of flow chemistry in biocatalytic processes has primarily been driven by a growing interest in process intensification and green chemistry. Continuous flow systems facilitate process intensification by decreasing residence times (often from hours to minutes), reducing the size of equipment required, and enabling production volume enhancement (Tamborini et al., Cell. 36(1):73-78 (2018)). From a green chemistry standpoint, these systems offer significant improvements in safety, waste generation, and energy efficiency due to heat management and mixing control (Newman and Jensen, Green Chem. (15):1456-1472 (2013)). The foregoing are incorporated by reference in their entirety.

The invention has many benefits over the prior art. It enables the efficient and economical production of glycans, such as complex polysaccharides, including by not limited to, HMOs using enzymes captured in modular flow processing cells. The flow cells may contain materials having large macropores or a high magnetic surface area for BNC immobilization. Flexible compositions for sintered magnetic scaffolds can be made with any meltable thermoplastics and magnetic material composition. The flow cells can have one or multiple enzyme systems that may be pieced together for particular sugar manufacturing processes.

A solution to combining biocatalysis and continuous flow systems is with functionalized flow cells. Biocatalytic flow cells are scaffolds containing immobilized enzymes for use in reactors such as continuous stirred tank reactors (CSTRs) and packed bed reactors (PBRs). Both types of reactors are known in the art but are primarily chosen based on the type of immobilization used. With a total market value of $5.8B in 2010, immobilized enzymes are used in a diverse range of large-scale processes including high fructose corn syrup production (10⁷ tons/year), transesterification of food oils (10⁵ tons/year), biodiesel synthesis (10⁴ tons/year), and chiral resolution of alcohols and amines (10 3 tons/year) (DiCosimo et al., Chem. Soc. Rev. (42):6437-6474 (2013), incorporated by reference herein in its entirety). These systems allow for improved downstream process management for enzymatic systems compared to batch reactors in terms of in-line control, enzyme reuse, and production scalability.

For the foregoing reasons, the inventions described herein provide biocatalytic systems for small-to-large scale manufacturing using BNCs in scaffolds that are shaped by 3D printing. In some embodiments, the biocatalytic systems are continuous flow.

Scaffolds may comprise cross-linked water-insoluble polymers and an approximately uniform distribution of embedded magnetic microparticles (MMP). The scaffolds may contain thermoset resins including Epoxy resins, Polyesters, Polyurethanes, Melamine resins, Vinyl esters, Silicones (polysiloxanes), Furan resins, Polyurea, Phenolic resins, phenol-formaldehyde, Urea-formaldehyde, Diallyl-phthalate (DAP), Benzoxazine, Polyimides and bismaleimides, Cyanate esters can be used. By combining natural or engineered enzymes, and in some embodiments with cofactors and cofactor recycling systems, the scaffold technology disclosed herein allows one to quickly translate innovation in biocatalysis to innovation in production for batch and flow processes. The magnetic powders are suitable for use flow chemistry applications such as pack-bed reactors.

Glycans obtained according to this invention may be used as components in the synthesis of any glycan-containing compound. With the ability to immobilize any enzymes for any processes, the materials functionalized with enzymes, or enzyme systems, have applications for the production of pharmaceuticals, biologicals, actives nutraceutical, actives cosmeceutical and food ingredients.

Self-assembled mesoporous nanoclusters comprising entrapped enzymes are highly active and robust. The technology is a powerful blend of biochemistry, nanotechnology, and bioengineering at three integrated levels of organization: Level 1 is the self-assembly of enzymes with magnetic nanoparticles (MNP) for the synthesis of magnetic mesoporous nanoclusters. This level uses a mechanism of molecular self-entrapment to immobilize and stabilize enzymes. Level 2 is the stabilization of the MNPs into other matrices. Level 3 is product conditioning and packaging for Level 1+2 delivery. The assembly of magnetic nanoparticles adsorbed to enzyme is herein also referred to as a “bionanocatalyst” (BNC).

The clusters may be magnetically templated onto shapeable magnetic scaffolds.

MNPs allow for a broader range of operating conditions such as temperature, ionic strength and pH. The size and magnetization of the MNPs affect the formation and structure of the NPs, all of which have a significant impact on the activity of the entrapped enzymes. By virtue of their surprising resilience under various reaction conditions, MNPs can be used as improved enzymatic or catalytic agents where other such agents are currently used. Furthermore, they can be used in other applications where enzymes have not yet been considered or found applicable.

The BNC contains mesopores that are interstitial spaces between the magnetic nanoparticles. The enzymes are preferably embedded or immobilized within at least a portion of mesopores of the BNC. As used herein, the term “magnetic” encompasses all types of useful magnetic characteristics, including permanent magnetic, superparamagnetic, paramagnetic, ferromagnetic, and ferrimagnetic behaviors.

The magnetic nanoparticle or BNC has a size in the nanoscale, i.e., generally no more than 500 nm. As used herein, the term “size” can refer to a diameter of the magnetic nanoparticle when the magnetic nanoparticle is approximately or substantially spherical. In a case where the magnetic nanoparticle is not approximately or substantially spherical (e.g., substantially ovoid or irregular), the term “size” can refer to either the longest the dimension or an average of the three dimensions of the magnetic nanoparticle. The term “size” may also refer to an average of sizes over a population of magnetic nanoparticles (i.e., “average size”).

In different embodiments, the magnetic nanoparticle has a size of precisely, about, up to, or less than, for example, 500 nm, 400 nm, 300 nm, 200 nm, 100 nm, 50 nm, 40 nm, 30 nm, 25 nm, 20 nm, 15 nm, 10 nm, 5 nm, 4 nm, 3 nm, 2 nm, or 1 nm, or a size within a range bounded by any two of the foregoing exemplary sizes.

In the BNC, the individual magnetic nanoparticles can be considered to be primary nanoparticles (i.e., primary crystallites) having any of the sizes provided above. The aggregates of nanoparticles in a BNC are larger in size than the nanoparticles and generally have a size (i.e., secondary size) of at least about 5 nm. In different embodiments, the aggregates have a size of precisely, about, at least, above, up to, or less than, for example, 5 nm, 8 nm, 10 nm, 12 nm, 15 nm, 20 nm, 25 nm, 30 nm, 35 nm, 40 nm, 45 nm, 50 nm, 60 nm, 70 nm, 80 nm, 90 nm, 100 nm, 150 nm, 200 nm, 300 nm, 400 nm, 500 nm, 600 nm, 700 nm, or 800 nm, or a size within a range bounded by any two of the foregoing exemplary sizes.

Typically, the primary and/or aggregated magnetic nanoparticles or BNCs thereof have a distribution of sizes, i.e., they are generally dispersed in size, either narrowly or broadly dispersed. In different embodiments, any range of primary or aggregate sizes can constitute a major or minor proportion of the total range of primary or aggregate sizes. For example, in some embodiments, a particular range of primary particle sizes (for example, at least about 1, 2, 3, 5, or 10 nm and up to about 15, 20, 25, 30, 35, 40, 45, or 50 nm) or a particular range of aggregate particle sizes (for example, at least about 5, 10, 15, or 20 nm and up to about 50, 100, 150, 200, 250, or 300 nm) constitutes at least or above about 50%, 60%, 70%, 80%, 90%, 95%, 98%, 99%, or 100% of the total range of primary particle sizes. In other embodiments, a particular range of primary particle sizes (for example, less than about 1, 2, 3, 5, or 10 nm, or above about 15, 20, 25, 30, 35, 40, 45, or 50 nm) or a particular range of aggregate particle sizes (for example, less than about 20, 10, or 5 nm, or above about 25, 50, 100, 150, 200, 250, or 300 nm) constitutes no more than or less than about 50%, 40%, 30%, 20%, 10%, 5%, 2%, 1%, 0.5%, or 0.1% of the total range of primary particle sizes.

The aggregates of magnetic nanoparticles (i.e., “aggregates”) or BNCs thereof can have any degree of porosity, including a substantial lack of porosity depending upon the quantity of individual primary crystallites they are made of In particular embodiments, the aggregates are mesoporous by containing interstitial mesopores (i.e., mesopores located between primary magnetic nanoparticles, formed by packing arrangements). The mesopores are generally at least 2 nm and up to 50 nm in size. In different embodiments, the mesopores can have a pore size of precisely or about, for example, 2, 3, 4, 5, 10, 12, 15, 20, 25, 30, 35, 40, 45, or 50 nm, or a pore size within a range bounded by any two of the foregoing exemplary pore sizes. Similar to the case of particle sizes, the mesopores typically have a distribution of sizes, i.e., they are generally dispersed in size, either narrowly or broadly dispersed. In different embodiments, any range of mesopore sizes can constitute a major or minor proportion of the total range of mesopore sizes or of the total pore volume. For example, in some embodiments, a particular range of mesopore sizes (for example, at least about 2, 3, or 5, and up to 8, 10, 15, 20, 25, or 30 nm) constitutes at least or above about 50%, 60%, 70%, 80%, 90%, 95%, 98%, 99%, or 100% of the total range of mesopore sizes or of the total pore volume. In other embodiments, a particular range of mesopore sizes (for example, less than about 2, 3, 4, or 5 nm, or above about 10, 15, 20, 25, 30, 35, 40, 45, or 50 nm) constitutes no more than or less than about 50%, 40%, 30%, 20%, 10%, 5%, 2%, 1%, 0.5%, or 0.1% of the total range of mesopore sizes or of the total pore volume.

The magnetic nanoparticles can have any of the compositions known in the art. In some embodiments, the magnetic nanoparticles are or include a zerovalent metallic portion that is magnetic. Some examples of such zerovalent metals include cobalt, nickel, and iron, and their mixtures and alloys. In other embodiments, the magnetic nanoparticles are or include an oxide of a magnetic metal, such as an oxide of cobalt, nickel, or iron, or a mixture thereof. In some embodiments, the magnetic nanoparticles possess distinct core and surface portions. For example, the magnetic nanoparticles may have a core portion composed of elemental iron, cobalt, or nickel and a surface portion composed of a passivating layer, such as a metal oxide or a noble metal coating, such as a layer of gold, platinum, palladium, or silver. In other embodiments, metal oxide magnetic nanoparticles or aggregates thereof are coated with a layer of a noble metal coating. The noble metal coating may, for example, reduce the number of charges on the magnetic nanoparticle surface, which may beneficially increase dispersibility in solution and better control the size of the BNCs. The noble metal coating protects the magnetic nanoparticles against oxidation, solubilization by leaching or by chelation when chelating organic acids, such as citrate, malonate, or tartrate are used in the biochemical reactions or processes. The passivating layer can have any suitable thickness, and particularly, at least, up to, or less than, about for example, 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or 10 nm, or a thickness in a range bounded by any two of these values.

Magnetic materials useful for the invention are well-known in the art. Non-limiting examples comprise ferromagnetic and ferromagnetic materials including ores such as iron ore (magnetite or lodestone), cobalt, and nickel. In other embodiments, rare earth magnets are used. Non-limiting examples include neodymium, gadolinium, sysprosium, samarium-cobalt, neodymium-iron-boron, and the like. In yet further embodiments, the magnets comprise composite materials. Non-limiting examples include ceramic, ferrite, and alnico magnets. In preferred embodiments, the magnetic nanoparticles have an iron oxide composition. The iron oxide composition can be any of the magnetic or superparamagnetic iron oxide compositions known in the art, e.g., magnetite (Fe₃O₄), hematite (α-Fe₂O₃), maghemite (γ-Fe₂O₃), or a spinel ferrite according to the formula AB₂O₄, wherein A is a divalent metal (e.g., Xn²+, Ni²+, Mn²⁺, Co²⁺, Ba²⁺, Sr²⁺, or combination thereof) and B is a trivalent metal (e.g., Fe³⁺, Cr³⁺, or combination thereof).

The individual magnetic nanoparticles or aggregates thereof or BNCs thereof possess any suitable degree of magnetism. For example, the magnetic nanoparticles, BNCs, or BNC scaffold assemblies can possess a saturated magnetization (Ms) of at least or up to about 5, 10, 15, 20, 25, 30, 40, 45, 50, 60, 70, 80, 90, or 100 emu/g. The magnetic nanoparticles, BNCs, or BNC-scaffold assemblies preferably possess a permanent magnetization (Mr) of no more than (i.e., up to) or less than 5 emu/g, and more preferably, up to or less than 4 emu/g, 3 emu/g, 2 emu/g, 1 emu/g, 0.5 emu/g, or 0.1 emu/g. The surface magnetic field of the magnetic nanoparticles, BNCs, or BNC-scaffold assemblies can be about or at least, for example, about 0.5, 1, 5, 10, 50, 100, 200, 300, 400, 500, 600, 700, 800, 900, or 1000 Gauss (G), or a magnetic field within a range bounded by any two of the foregoing values. If microparticles are included, the microparticles may also possess any of the above magnetic strengths.

The magnetic nanoparticles or aggregates thereof can be made to adsorb a suitable amount of enzyme, up to or below a saturation level, depending on the application, to produce the resulting BNC. In different embodiments, the magnetic nanoparticles or aggregates thereof may adsorb about, at least, up to, or less than, for example, 1, 5, 10, 15, 20, 25, or 30 pmol/m2 of enzyme. Alternatively, the magnetic nanoparticles or aggregates thereof may adsorb an amount of enzyme that is about, at least, up to, or less than, for example, about 10%, 20%, 30%, 40%, 50%, 60%, 70%, 80%, 90%, or 100% of a saturation level.

The magnetic nanoparticles or aggregates thereof or BNCs thereof possess any suitable pore volume. For example, the magnetic nanoparticles or aggregates thereof can possess a pore volume of about, at least, up to, or less than, for example, about 0.01, 0.05, 0.1, 0.15, 0. 2, 0.25, 0.3, 0.35, 0.4, 0.45, 0.5, 0.55, 0.6, 0.65, 0.7, 0.75, 0.8, 0.85, 0.9, 0.95, or 1 cm3/g, or a pore volume within a range bounded by any two of the foregoing values.

The magnetic nanoparticles or aggregates thereof or BNCs thereof possess any suitable specific surface area. For example, the magnetic nanoparticles or aggregates thereof can have a specific surface area of about, at least, up to, or less than, for example, about 50, 60, 70, 80, 90, 100, 110, 120, 130, 140, 150, 160, 170, 180, 190, or 200 m²/g.

The magnetic macroporous matrix material according to this invention has a size of precisely, about, up to, or less than, for example, 100-1000, 50-100, 10-50 μm, or 5-10, 1000, 900, 800, 700, 600, 500, 400, 300, 200, 100, 90, 80, 70, 60, 50, 40, 30, 20, 10, 5, less than 5, greater than 100, an average size of 150, an average size of 75, an average size of 40, an average size of 20 or an average size of about 15. In certain embodiments, the material has an average particle diameter of precisely, about, up to, or less than, 20-40 μm, 20 μm, or 40 μm. In certain embodiments, the material has a tight size distribution of an average particle diameter of either 20 μm or 40 μm.

MNPs, macroporous powders, scaffolds, their structures, organizations, suitable enzymes, and uses are described in WO2012/122437, WO2014/055853, WO2016/186879, WO2017/011292, WO2017/180383, WO2018/34877, WO2018/102319, WO2020/051159, and WO2020/69227, incorporated by reference herein in their entirety.

In some embodiments, the methods described herein use recombinant cells that express the enzymes used in the invention. Recombinant DNA technology is known in the art. In some embodiments, cells are transformed with expression vectors such as plasmids that express the enzymes. In other embodiments, the vectors have one or more genetic signals, e.g., for transcriptional initiation, transcriptional termination, translational initiation and translational termination. Here, nucleic acids encoding the enzymes may be cloned in a vector so that they are expressed when properly transformed into a suitable host organism. Suitable host cells may be derived from bacteria, fungi, plants, or animals as is well-known in the art.

Although BNCs (Level 1) provide the bulk of enzyme immobilization capability, they are sometimes too small to be easily captured by standard-strength magnets. Thus, sub-micrometric magnetic materials (Level 2) are used to provide bulk magnetization and added stability to Level 1. Commercially available free magnetite powder, with particle sizes ranging from 50-500 nm, is highly hydrophilic and tends to stick to plastic and metallic surfaces, which, over time, reduces the effective amount of enzyme in a given reactor system. In addition, powdered magnetite is extremely dense, thus driving up shipping costs. It is also rather expensive—especially at particle sizes finer than 100 nm. To overcome these limitations, low-density hybrid materials consisting of magnetite, non-water-soluble cross-linked polymers such as poly(vinylalcohol) (PVA) and carboxymethylcellulose (CMC), have been developed. These materials are formed by freeze-casting and freeze-drying water-soluble polymers followed by cross-linking. These materials have reduced adhesion to external surfaces, require less magnetite, and achieve Level 1 capture that is at least comparable to that of pure magnetite powder.

In one embodiment, the continuous macroporous scaffold has a cross-linked polymeric composition. The polymeric composition can be any of the solid organic, inorganic, or hybrid organic-inorganic polymer compositions known in the art, and may be synthetic or a biopolymer that acts as a binder. Preferably, the polymeric s scaffold does not dissolve or degrade in water or other medium in which the hierarchical catalyst is intended to be used. Some examples of synthetic organic polymers include the vinyl addition polymers (e.g., polyethylene, polypropylene, polystyrene, polyacrylic acid or polyacrylate salt, polymethacrylic acid or polymethacrylate salt, poly(methylmethacrylate), polyvinyl acetate, polyvinyl alcohol, and the like), fluoropolymers (e.g., polyvinylfluoride, polyvinylidenefluoride, polytetrafluoroethylene, and the like), the epoxides (e.g., phenolic resins, resorcinol-formaldehyde resins), the polyamides, the polyurethanes, the polyesters, the polyimides, the polybenzimidazoles, and copolymers thereof. Some examples of biopolymers include the polysaccharides (e.g., cellulose, hemicellulose, xylan, chitosan, inulin, dextran, agarose, and alginic acid), polylactic acid, and polyglycolic acid. In the particular case of cellulose, the cellulose may be microbial- or algae-derived cellulose. Some examples of inorganic or hybrid organic-inorganic polymers include the polysiloxanes (e.g., as prepared by sol gel synthesis, such as polydimethylsiloxane) and polyphosphazenes. In some embodiments, any one or more classes or specific types of polymer compositions provided above are excluded as macroporous scaffolds.

### EMBODIMENTS

1. A module comprising a magnetic macroporous matrix material comprising self-assembled mesoporous aggregates of magnetic nanoparticles magnetically entrapping an immobilized glycan synthesis enzyme.

2. The module according to embodiment 1 wherein said self-assembled mesoporous aggregates comprise a single glycan synthesis enzyme or a combination of glycan synthesis enzymes.

3. The module according to embodiment 1 or embodiment 2 wherein said material comprises a sugar activation enzyme, a sugar extension enzyme, a reagent regeneration enzyme, a sugar functionalization enzyme, a sugar support enzyme, or a sugar removal enzyme.

4. The module according to embodiment 1 or embodiment 2, wherein said material comprises a glycan synthesis enzyme to activate and to transfer a sugar.

5. The module according to embodiment 3, wherein said regeneration enzyme is a nucleotide regeneration enzyme or a cofactor regeneration enzyme.

6. The module according to embodiment 3, wherein said sugar functionalization enzyme either phosphorylates or sulfates glycans.

7. The module according to embodiment 3, wherein said material comprises an elementary module (EM), wherein said EM is EM-1, EM-2, EM-3, EM-4, EM-5, or EM-6.

8. The module according to embodiment 3, wherein said material comprises a system enzyme module (SM), wherein said SM is SM-1, SM-2, SM-3, SM-4, SM-5, SM-6, SM-7, SM-8, SM-9, SM-10, or SM-11.

9. The module according to any one of embodiments 1-7, wherein said material is in a flow cell.

10. The module according to any one of embodiments 1-7, wherein said material is in a pack bed.

11. The module according to any one of embodiments 1-9, wherein said magnetic macroporous material comprises a metal oxide.

12. The module according to any one of embodiments 1-9, wherein said magnetic macroporous material comprises metal oxide complex.

13. The module according to any one of embodiments 1-9, wherein said macroporous material comprises a thermoplastic polymer, and cross-linked polymer, or a thermoset resin.

14. The module according to any one of embodiments 1-9, wherein said macroporous material comprises strontium ferrite.

15. The module according to any one of embodiments 1-13, wherein said nanoparticles comprise magnetite (Fe₃O₄) or maghemite (Fe₂O₃).

16. The module according to any one of embodiments 1-13, wherein said nanoparticles comprise FeCl₂*4H₂O (Iron (II) chloride tetrahydrate or FeCl₃*6H₂O (Iron (III) chloride hexahydrate.

17. A device for producing a glycan, comprising a module according to any one of embodiments 1-16, wherein said module modifies a glycan substrate to produce a glycan

18. A device for producing a glycan, comprising a first module according to any one of embodiments 1-16 and a second module, according to any one of embodiments 1-16 wherein a first substrate is introduced into said first module to create a modified first substrate; wherein said first modified substrate is a second substrate introduced into said second module to produce a glycan.

19. The device according to embodiment 18, further comprising a third module, wherein said glycan is introduced into said third module to create a third modified substrate.

20. The device according to embodiment 19, further comprising a fourth module, wherein said third modified substrate is introduced into said fourth module to create a fourth modified substrate.

21. The device according to embodiment 20, further comprising a fifth module, wherein said fourth modified substrate is introduced into said fifth module to create a fifth modified substrate.

22. The device according to embodiment 21, further comprising a sixth module, wherein said fifth modified substrate is introduced into said sixth module to create a sixth modified substrate.

23. A method for producing a glycan, comprising modifying a glycan substrate with a module according to any one of embodiments 1-15, wherein said module modifies a glycan substrate to produce a glycan.

24. The method according to embodiment 23, wherein the modifying is a batch process.

25. The method according to embodiment 23, wherein the modifying is a flow process.

26. The method according to embodiment 23, wherein the modifying is a continuous flow process.

27. The method according to any one of embodiments 23-26, wherein the module comprises a sugar activation enzyme.

28. The method according to any one of embodiments 23-26, wherein said sugar activation enzyme activates fucose, sialic acid, N-Acetylglucosamine (GlcNAc) or N-Acetylneuraminic acid (Neu5Ac).

29. The method according to embodiment 27, wherein said sugar activation enzyme is a natural or synthetic enzyme that is a kinase and a nucleotide transferase or a natural or synthetic fusion enzyme that integrates both functions into one enzyme.

30. The method according to embodiment 29, wherein said sugar activation enzyme is combination of Galactokinase (GalK, EC 2.7.1.6) and Galactose-1-phosphate uridyltransferase (Gal-1-phosphate-UDP T, EC 2.7.7.64).

31. The method according to embodiment 30, wherein the sugar activation enzyme is BiGalK/BiUSP from Bifidobacterium infantis.

32. The method according to embodiment 29, wherein the sugar activation enzyme is a combination of N-acetylglucosamine kinase (G1cNAcK, EC 2.7.1.162) and N-Acetylglucosamine-1-phosphate-uridyltransferase (GlmU, EC 2.7.7.23) that catalyzes the conversion of GlcNAc to UDP-GlcNAc.

33. The method according to embodiment 32, wherein the sugar activation enzyme is BiNahK from Bifidobacterium infantis and HmG1mU from Helicobacter mustelae.

34. The method according to embodiment 32, wherein the sugar activation enzyme is BiNahK from Bifidobacterium infantis, and CjG1mU from Campylobacter jejuni.

35. The method according to embodiment 29, wherein said sugar activation enzyme is a combination of Fucokinase (fucK, EC 2.7.1.52) and Fucose-1-phosphate-guanylyltransferase (Fuc-1P-GDP T, EC 2.7.7.30) that catalyzes the conversion of L-fucose to GDP-fucose.

36. The method according to embodiment 29, wherein said sugar activation enzyme is a combination of Glucuronokinase (GlcA K, EC 2.7.1.43) and Glucurono-1-phosphate-uridyltransferase (GlcA-1-phosphate-UDP T, EC 2.7.7.44) that catalyzes the conversion of GlcA to UDP-GlcA.

37. The method according to embodiment 29, wherein said sugar activation enzyme is a combination of Glucokinase (Glc K, EC 2.7.1.2) and Glucose-1-phosphate-uridyltransferase (Glc-1-phosphate-UDP T, EC 2.7.7.9) that catalyzes the conversion of Glc to UDP-Glc.

38. The method according to embodiment 29, wherein said sugar activation enzyme is a combination of Mannokinase (Man K, EC 2.7.1.7) and Mannose-1-phosphate-guanyltransferase (Man-1-phosphate-GDP T, EC 2.7.7.13, 2.7.7.22) that catalyzes the conversion of Man to GDP-Man.

39. The method according to embodiment 29, wherein said sugar activation enzyme is a combination of Rhamnokinase (Rha K, EC 2.7.1.5) and Rhamnose-1-phosphate-uridyltransferase (Rha-1-phosphate-UDP T) that catalyzes the conversion of Rha to UDP-Rha.

40. The method according to embodiment 29, wherein said sugar activation enzyme is a natural fusion enzyme (Bifunctional fucokinase/L-fucose-1-P-guanylyltransferase=FKP, EC 2.7.1.52/2.7.7.30) that catalyzes the conversion of L-fucose to GDP-fucose.

41. The method according to embodiment 40, wherein said sugar activation enzyme is a BfFKP from Bacteroides fragilis.

42. The method according to embodiment 29, wherein said sugar activation enzyme is a synthetic fusion enzyme combining N-acetylglucosamine kinase (G1cNAcK, EC 2.7.1.162) and N-Acetylglucosamine-1-phosphate-uridyltransferase (GlmU, EC 2.7.7.23) that catalyzes the conversion of GlcNAc to UDP-GlcNAc.

43. The method according to embodiment 42, wherein said sugar activation enzyme is a BINahK-EcGlmU from Bifidobacterium infantis and Escherichia coli.

44. The method according to embodiment 27, wherein said sugar activation enzyme is an oxidase.

45. The method according to embodiment 44, wherein said oxidase is UDP-Glc-6-dehydrogenase, EC 1.1.1.22.

46. The method according to embodiment 27, wherein said sugar activation enzyme produces activated sugars via isomerization from a structurally related activated sugar.

47. The method according to embodiment 46, wherein said sugar activation enzyme is an epimerase UDP-Gal-4-epimerase (EC 5.1.3.2).

48. The method according to embodiment 47, wherein said epimerase is EcGalE from Escherichia coli.

49. The method according to embodiment 47, wherein said epimerase is StGalE from Streptococcus thermophilus

50. The method according to embodiment 27, wherein sugar activation enzyme is a GlycoSynthetases.

51. The method according to embodiment 50, wherein the GlycoSynthetase is CMP-sialic acid synthetase=CSS, (EC 2.7.7.43).

52. The method according to embodiment 51, wherein the GlycoSynthetase is NmCSS from Neisseria meningitides.

53. The method according to embodiment 50, wherein the GlycoSynthetase is 3-deoxy-manno-octulosonate cytidyl transferase synthetase=CMP-KDO synthetase=CKS, EC 2.7.7.38).

54. The method according to embodiment 27, wherein sugar activation enzyme is a kinase.

55. The method according to embodiment 54, wherein the kinase is Galactokinase, EC 2.7.1.6.

56. The method according to embodiment 55, wherein the Galactokinase is BiGalK from Bifidobacterium infantis.

57. The method according to embodiment 54, wherein the kinase is Glucokinase, EC 2.7.1.2.

58. The method according to any one of embodiments 23-26, wherein the glycan synthesis enzyme is a sugar extension (transfer) enzyme.

59. The method according to embodiment 58, wherein the sugar extension enzyme is a sugar phosphorylase.

60. The method according to embodiment 59, wherein the sugar extension enzyme is phosphorylase (1,3-β-galactosyl-N-acetylhexosamine phosphorylase=Galacto-N-biose phosphorylase=GalHexNAcP, EC 2.4.1.211) that catalyzes the conversion of Gal-1P (sugar donor) to GlcNAc with a β-1,3 linkage.

61. The method according to embodiment 60, wherein the sugar extension enzyme is BiGalHexNAcP from Bifidobacterium infantis.

62. The method according to embodiment 58, wherein the sugar extension enzyme is a GlycoSynthetase.

63. The method according to embodiment 62, wherein the GlycoSynthetase is fucosidase (EC 3.2.1.51).

64. The method according to embodiment 63, wherein the fucosidase is α-1,2-fucosidase (EC 3.2.1.63)

65. The method according to embodiment 63, wherein the fucosidase is α-1,3-fucosidase (EC 3.2.1.111)

66. The method according to embodiment 63, wherein the fucosidase is α-1,4-fucosidase (EC not known).

67. The method according to embodiment 62, wherein the GlycoSynthetase is a neuraminidase (sialidase, EC 3.2.1.18).

68. The method according to embodiment 67, wherein the neuraminidase is α-2,3-neuraminidase

69. The method according to embodiment 67, wherein the neuraminidase is α-2,6-neuraminidase

70. The method according to embodiment 67, wherein the neuraminidase is α-2,8-neuraminidase

71. The method according to embodiment 62, wherein the GlycoSynthetase is a β-N-acetylhexosaminidase (β-N-acetylglucosaminidase, EC 3.2.1.52).

72. The method according to embodiment 71, wherein the aminidase is β-1,3-N-acetylglucosaminidase.

73. The method according to embodiment 72, wherein the aminidase is Bbh1 from Bifidobacterium bifidum.

74. The method according to embodiment 62, wherein the GlycoSynthetase is a β-N-acetylgalactosaminidase (EC 3.2.1.53).

75. The method according to embodiment 74, wherein the aminidase is β-1,4-N-acetylgalactosaminidase.

76. The method according to embodiment 62, wherein the GlycoSynthetase is an α or β-galactosidase (EC 3.2.1.22, 3.2.1.23).

77. The method according to embodiment 76, wherein the galactosidase is α-1,2-galactosidase.

78. The method according to embodiment 76, wherein the galactosidase is α-1,3-galactosidase.

79. The method according to embodiment 76, wherein the galactosidase is α-1,4-galactosidase.

80. The method according to embodiment 76, wherein the galactosidase is α-1,6-galactosidase.

81. The method according to embodiment 76, wherein the galactosidase is β-1,3-galactosidase.

82. The method according to embodiment 76, wherein the galactosidase is β-1,4-galactosidase.

83. The method according to embodiment 76, wherein the galactosidase is β-1,6-galactosidase.

84. The method according to embodiment 62, wherein the GlycoSynthetase is a β-glucuronidase.(EC 3.2.1.31).

85. The method according to embodiment 84, wherein the glucuronidase is β-1,3-glucuronidase.

86. The method according to embodiment 62, wherein the GlycoSynthetase an α or β-glucosidase (EC 3.2.1.20, 3.2.1.21).

87. The method according to embodiment 86, wherein the glucosidase is β-1,3-glucosidase.

88. The method according to embodiment 86, wherein the glucosidase is β-1,4-glucosidase.

89. The method according to embodiment 86, wherein the glucosidase is β-1,6-glucosidase.

90. The method according to embodiment 86, wherein the glucosidase is α-1,4-glucosidase.

91. The method according to embodiment 62, wherein the GlycoSynthetase is an a or β-mannosidase (EC 3.2.1.24, 3.2.1.25).

92. The method according to embodiment 91, wherein the mannosidase is α-1,2-mannosidase.

93. The method according to embodiment 91, wherein the mannosidase is α-1,3-mannosidase.

94. The method according to embodiment 91, wherein the mannosidase is β-1,3-mannosidase.

95. The method according to embodiment 91, wherein the mannosidase is β-1,4-mannosidase.

96. The method according to embodiment 62, wherein the GlycoSynthetase is an β-xylosidase (EC 3.2.1.37).

97. The method according to embodiment 96, wherein the xylosidase is β-1,4-xylosidase.

98. The method according to embodiment 62, wherein the GlycoSynthetase is an endo specific glycosynthetase that transfers sugar oligosaccharides to acceptor glycans.

99. The method according to embodiment 98, wherein the glycosynthetase is arabinogalactan endo-β-1,4-galactanase (EC 3.2.1.89).

100. The method according to embodiment 98, wherein the glycosynthetase is mannosyl-glycoprotein endo-β-N-acetylglucosaminidase (EC 3.2.1.96).

101. The method according to embodiment 98, wherein the glycosynthetase is EM-2-2-10-3. endo-α-N-acetylgalactosaminidase (EC 3.2.1.97).

102. The method according to embodiment 98, wherein the glycosynthetase is blood-group-substance endo-1,4-β-galactosidase (EC 3.2.1.102).

103. The method according to embodiment 98, wherein the glycosynthetase keratan-sulfate endo-1,4-β-galactosidase (EC 3.2.1.103).

104. The method according to embodiment 98, wherein the glycosynthetase glycoprotein endo-α-1,2-mannosidase (EC 3.2.1.130).

105. The method according to embodiment 98, wherein the glycosynthetase lacto-N-biosidase (EC 3.2.1.140).

106. The method according to embodiment 105, wherein the glycosynthetase LnbB from Bifidobacterium bifidum.

107. The method according to embodiment 98, wherein the glycosynthetase mannosylglycoprotein endo-P-mannosidase (EC 3.2.1.152).

108. The method according to embodiment 62, wherein the sugar extension enzyme is a GlycoTransferase.

109. The method according to embodiment 108, wherein the GlycoTransferase is a fucosyltransferase.

110. The method according to embodiment 109, wherein the fucosyltransferase is α-1,2-fucosyltransferase (EC 2.4.1.69).

111. The method according to embodiment 110, wherein the fucosyltransferase is Te2FT from Thermosynechococcus elongatus.

112. The method according to embodiment 110, wherein the fucosyltransferase is WbgL from Escherichia coli.

113. The method according to embodiment 110, wherein the fucosyltransferase is HmFucT from Helicobacter mustelae.

114. The method according to embodiment 110, wherein the fucosyltransferase is FUT1 from Homo sapiens.

115. The method according to embodiment 110, wherein the fucosyltransferase is FUT2 from Homo sapiens.

116. The method according to embodiment 109, wherein the fucosyltransferase is α-1,3-fucosyltransferase (EC 2.4.1.152, EC 2.4.1.214).

117. The method according to embodiment 116, wherein the fucosyltransferase is HpFucT from Helicobacter pylori.

118. The method according to embodiment 116, wherein the fucosyltransferase Bf1,3FT from Bacteroides fragilis.

119. The method according to embodiment 116, wherein the fucosyltransferase is Hp3/4FT from Helicobacter pylori.

120. The method according to embodiment 116, wherein the fucosyltransferase FUT3 from Homo sapiens.

121. The method according to embodiment 116, wherein the fucosyltransferase FUT4 from Homo sapiens.

122. The method according to embodiment 116, wherein the fucosyltransferase FUT5 from Homo sapiens.

123. The method according to embodiment 116, wherein the fucosyltransferase FUT6 from Homo sapiens.

124. The method according to embodiment 116, wherein the fucosyltransferase FUT7 from Homo sapiens.

125. The method according to embodiment 116, wherein the fucosyltransferase FUT9 from Homo sapiens.

126. The method according to embodiment 116, wherein the fucosyltransferase FUT11 from Homo sapiens.

127. The method according to embodiment 109, wherein the fucosyltransferase is α-1,4-fucosyltransferase (EC 2.4.1.65).

128. The method according to embodiment 127, wherein the fucosyltransferase is Hp3/4FT from Helicobacter pylori.

129. The method according to embodiment 127, wherein the fucosyltransferase is FUT2 from Homo sapiens.

130. The method according to embodiment 109, wherein the fucosyltransferase is α-1,6-fucosyltransferase (EC 2.4.1.68).

131. The method according to embodiment 130, wherein the fucosyltransferase is EM-2-3-1-4-1 FUT8 from Homo sapiens.

132. The method according to embodiment 108, wherein the GlycoTransferase is sialyltransferase.

133. The method according to embodiment 132, wherein the sialyltransferase is α-2,3-sialyltransferase (EC 2.4.99.4, EC 2.4.99.6, EC 2.4.99.7, EC 2.4.99.9).

134. The method according to embodiment 133, wherein the sialyltransferase is PmST1 (wild type and M144D mutant) from Pasteurella multocida.

135. The method according to embodiment 133, wherein the sialyltransferase is NmST1-NmCSS fusion from Neisseria meningitidis.

136. The method according to embodiment 133, wherein the sialyltransferase is ST3GAL1 from Homo sapiens.

137. The method according to embodiment 133, wherein the sialyltransferase is ST3GAL2 from Homo sapiens.

138. The method according to embodiment 133, wherein the sialyltransferase is ST3GAL3 from Homo sapiens.

139. The method according to embodiment 133, wherein the sialyltransferase is ST3GAL4 from Homo sapiens.

140. The method according to embodiment 133, wherein the sialyltransferase is ST3GAL5 from Homo sapiens.

141. The method according to claim embodiment 133, wherein the sialyltransferase is ST3GAL6 from Homo sapiens.

142. The method according to embodiment 132, wherein the sialyltransferase is α-2,6-sialyltransferase (EC 2.4.99.1, EC 2.4.99.3).

143. The method according to embodiment 142, wherein the sialyltransferase is Pd26ST from Photobacterium damsel.

144. The method according to embodiment 142, wherein the sialyltransferase is ST6GAL1 from Homo sapiens.

145. The method according to embodiment 142, wherein the sialyltransferase is ST6GAL2 from Homo sapiens.

146. The method according to embodiment 142, wherein the sialyltransferase is ST6GALNAC1 from Homo sapiens.

147. The method according to embodiment 142, wherein the sialyltransferase is ST6GALNAC2 from Homo sapiens.

148. The method according to embodiment 142, wherein the sialyltransferase is ST6GALNAC3 from Homo sapiens.

149. The method according to embodiment 142, wherein the sialyltransferase is ST6GALNAC4 from Homo sapiens.

150. The method according to embodiment 142, wherein the sialyltransferase is ST6GALNACS from Homo sapiens.

151. The method according to embodiment 142, wherein the sialyltransferase is ST6GALNAC6 from Homo sapiens. 152. The method according to embodiment 132, wherein the sialyltransferase is α-2,8-sialyltransferase (EC 2.4.99.8).

153. The method according to embodiment 152, wherein the sialyltransferase is α-2,3/8-sialyltransferase from Campylobacter jejuni.

154. The method according to embodiment 152, wherein the sialyltransferase is ST8SIA1 from Homo sapiens.

155. The method according to embodiment 152, wherein the sialyltransferase is. ST8SIA2 from Homo sapiens.

156. The method according to embodiment 152, wherein the sialyltransferase is ST8SIA3 from Homo sapiens.

157. E The method according to embodiment 152, wherein the sialyltransferase is ST8SIA4 from Homo sapiens.

158. The method according to embodiment 152, wherein the sialyltransferase is ST8SIA5 from Homo sapiens.

159. The method according to embodiment 108, wherein the GlycoTransferase is an N-acetylglucosaminyltransferase.

160. The method according to embodiment 159, wherein the GlycoTransferase is 13-1,3-N-acetylglucosaminyltransferase (EC 2.4.1.79, EC 2.4.1.149, EC 2.4.1.222).

161. The method according to embodiment 160 wherein the GlycoTransferase is HpLgtA form Helicobacter pylori.

162. The method according to embodiment 160 wherein the GlycoTransferase is NmLgtA form Neisseria meningitidis.

163. The method according to embodiment 160 wherein the GlycoTransferase is HP1105 from Helicobacter pylori.

164. The method according to embodiment 160 wherein the GlycoTransferase is B3GNT2 from Homo sapiens.

165. The method according to embodiment 160 wherein the GlycoTransferase is B3GNT3 from Homo sapiens.

166. The method according to embodiment 160 wherein the GlycoTransferase is B3GNT4 from Homo sapiens.

167. The method according to embodiment 160 wherein the GlycoTransferase is EM-2-3-3-1-7. B3GNT7 from Homo sapiens.

168. The method according to embodiment 160 wherein the GlycoTransferase is EM-2-3-3-1-8. B3GNT8 from Homo sapiens.

169. The method according to embodiment 160 wherein the GlycoTransferase is EM-2-3-3-1-9. B3GNT9 from Homo sapiens.

170. The method according to embodiment 159, wherein the GlycoTransferase is α-1,4-N-acetylglucosaminyltransferase (EC 2.4.1.223, EC 2.4.1.224).

171. The method according to embodiment 159, wherein the GlycoTransferase is 13-1,2-N-acetylglucosaminyltransferase (EC 2.4.1.101, EC 2.4.1.143).

172. The method according to embodiment 171, wherein the GlycoTransferase is MGAT1 (G1cNAcT-I) from Homo sapiens.

173. The method according to embodiment 171, wherein the GlycoTransferase is MGAT2 (G1cNAcT-II) from Homo sapiens.

174. The method according to embodiment 159, wherein the GlycoTransferase is 13-1,4-N-acetylglucosaminyltransferase (EC 2.4.1.144, EC 2.4.1.212).

175. The method according to embodiment 174, wherein the GlycoTransferase is EM-2-3-3-4-1. MGAT3 (G1cNAcT-III) from Homo sapiens.

176. The method according to embodiment 174, wherein the GlycoTransferase is EM-2-3-3-4-2. MGAT4A (G1cNAcT-IV) from Homo sapiens.

177. The method according to embodiment 174, wherein the GlycoTransferase is EM-2-3-3-4-3. MGAT4B (G1cNAcT-IV) from Homo sapiens.

178. The method according to embodiment 174, wherein the GlycoTransferase is EM-2-3-3-4-4. MGAT4C (G1cNAcT-IV) from Homo sapiens.

179. The method according to embodiment 159, wherein the GlycoTransferase is β-1,6-N-acetylglucosaminyltransferase (EC 2.4.1.102, EC 2.4.1.150, EC 2.4.1.155).

180. The method according to embodiment 179, wherein the GlycoTransferase is GCNT2A from Homo sapiens.

181. The method according to embodiment 179, wherein the GlycoTransferase is GCNT2B from Homo sapiens.

182. The method according to embodiment 179, wherein the GlycoTransferase is GCNT2C from Homo sapiens.

183. The method according to embodiment 179, wherein the GlycoTransferase is GCNT3 from Homo sapiens.

184. The method according to embodiment 179, wherein the GlycoTransferase is GCNT4 from Homo sapiens.

185. The method according to embodiment 179, wherein the GlycoTransferase is MGATS (G1cNACT-V) from Homo sapiens.

186. The method according to embodiment 108, wherein the GlycoTransferase an N-acetylgalactosyltransferase that catalyzes the transfer of an activated UDP-N-acetylgalactosamine (UDP-GalNAc) to Galactose.

187. The method according to embodiment 186, wherein the GlycoTransferase is α-1,3-N-acetylgalactosyltransferase (EC 2.4.1.40).

188. The method according to embodiment 187, wherein the GlycoTransferase is ABO from Homo sapiens.

189. The method according to embodiment 187, wherein the GlycoTransferase is BgtA from Helicobacter mustelae.

190. The method according to embodiment 186, wherein the GlycoTransferase EM-2-3-4-2. β-1,4-N-acetylgalactosyltransferase (EC 2.4.1.174, EC 2.4.1.175).

191. The method according to embodiment 190, wherein the GlycoTransferase B4GALNT3 from Homo sapiens.

192. The method according to embodiment 190, wherein the B4GALNT4 from Homo sapiens.

193. The method according to embodiment 108, wherein the GlycoTransferase is a galactosyltransferase.

194. The method according to embodiment 193, wherein the galactosyltransferase is 13-1,3-galactosyltransferase (EC 2.4.1.122, EC 2.4.1.134).

195. The method according to embodiment 194, wherein the galactosyltransferase is Cvβ3GalT from Chromobacterium violaceum.

196. The method according to embodiment 194, wherein the galactosyltransferase is WbgO from Escherichia coli.

197. The method according to embodiment 194, wherein the galactosyltransferase is CgtB from Campylobacter jejuni.

198. The method according to embodiment 194, wherein the galactosyltransferase is B3GALT1 from Homo sapiens.

199. The method according to embodiment 194, wherein the galactosyltransferase is. B3GALT2 from Homo sapiens.

200. The method according to embodiment 194, wherein the galactosyltransferase is B3GALT4 from Homo sapiens.

201. The method according to embodiment 194, wherein the galactosyltransferase is B3GALT5 from Homo sapiens.

202. The method according to embodiment 193, wherein the galactosyltransferase is β-1,4-galactosyltransferase (EC 2.4.1.22, EC 2.4.1.38, EC 2.4.1.90, EC 2.4.1.133).

203. The method according to embodiment 202, wherein the galactosyltransferase is NmLgtB from Neisseria meningitidis.

204. The method according to embodiment 202, wherein the galactosyltransferase is NmLgtB-StGalE from Neisseria meningitidis and Streptococcus thermophilus.

205. The method according to embodiment 202, wherein the galactosyltransferase is HpLgtB from Helicobacter pylori.

206. The method according to embodiment 202, wherein the galactosyltransferase is B4GALT1 from Homo sapiens.

207. The method according to embodiment 202, wherein the galactosyltransferase is B4GALT2 from Homo sapiens.

208. The method according to embodiment 202, wherein the galactosyltransferase is B4GALT3 from Homo sapiens.

209. The method according to embodiment 202, wherein the galactosyltransferase is B4GALT4 from Homo sapiens.

210. The method according to embodiment 202, wherein the galactosyltransferase is B4GALT5 from Homo sapiens.

211. The method according to embodiment 202, wherein the galactosyltransferase is B4GALT6 from Homo sapiens.

212. The method according to embodiment 202, wherein the galactosyltransferase is B4GALT7 from Homo sapiens.

213. The method according to embodiment 193, wherein the galactosyltransferase is α-1,3-galactosyltransferase (EC 2.4.1.37).

214. The method according to embodiment 213, wherein the galactosyltransferase is GTB (human proteins) synthetic gene expressed in E. Coli.

215. The method according to embodiment 213, wherein the galactosyltransferase is WbnL from E. Coli.

216. The method according to embodiment 193, wherein the galactosyltransferase is EM-2-3-5-4. α-1,6-galactosyltransferase (EC 2.4.1.241).

217. The method according to embodiment 108, wherein the GlycoTransferase is a glucuronic acid transferase.

218. The method according to embodiment 217, wherein the glucuronic acid transferase is β-1,3-glucuronic acid transferase (EC 2.4.1.135, EC 2.4.1.212, EC 2.4.1.226).

219. The method according to embodiment 217, wherein the glucuronic acid transferase is β-1,4-glucuronic acid transferase (EC 2.4.1.226).

220. The method according to embodiment 219, wherein the glucuronic acid transferase is B4GAT1 from Homo sapiens.

221. The method according to embodiment 108, wherein the GlycoTransferase is glucosyltransferase that catalyzes the transfer of activated UDP-Glucose (UDP-Glc) to GlcNAc, Mannose, Glucose or Gal.

222. The method according to embodiment 221, wherein the GlycoTransferase 13-1,2-glucosyltransferase (EC 2.4.1.208).

223. The method according to embodiment 221, wherein the GlycoTransferase 13-1,3-glucosyltransferase (EC 2.4.1.305).

224. The method according to embodiment 221, wherein the GlycoTransferase α-1,3-glucosyltransferase (EC 2.4.1.256, EC 2.4.1.265, EC 2.4.1.267).

225. The method according to embodiment 221, wherein the GlycoTransferase α-1,4-glucosyltransferase (EC 2.4.1.374).

226. The method according to embodiment 108, wherein the GlycoTransferase is a mannosyltransferase.

227. The method according to embodiment 108, wherein the mannosyltransferase is α-1,2-mannosyltransferase (EC 2.4.1.131, EC 2.4.1.259, EC 2.4.1.260, EC 2.4.1.270).

228. The method according to embodiment 108, wherein the mannosyltransferase is α-1,3-mannosyltransferase (EC 2.4.1.132, EC 2.4.1.252, EC 2.4.1.258).

229. The method according to embodiment 108, wherein the mannosyltransferase is. β-1,4-mannosyltransferase (EC 2.4.1.142, EC 2.4.1.251).

230. The method according to embodiment 108, wherein the mannosyltransferase is α-1,6-mannosyltransferase (EC 2.4.1.257, EC 2.4.1.260).

231. The method according to embodiment 108, wherein the GlycoTransferase is a rhamnosyltransferase.

232. The method according to embodiment 231, wherein the rhamnosyltransferase is α-1,3-rhamnosyltransferase (EC 2.4.1.159, EC 2.4.1.289).

233. The method according to embodiment 231, wherein the rhamnosyltransferase is α-1,4-rhamnosyltransferase (EC 2.4.1.3751).

234. The method according to embodiment 108, wherein the GlycoTransferase is a xylosyltransferase.

235. The method according to embodiment 234, wherein the xylosyltransferase is β-1,2-xylosyltransferase (EC 2.4.2.38).

236. The method according to embodiment 234, wherein the xylosyltransferase is α-1,6-xylosyltransferase (EC 2.4.2.39).

237. The method according to any one of embodiments 23-26, wherein the glycan synthesis enzyme is a reagent regeneration enzyme.

238. The method according to embodiment 237, wherein the reagent regeneration enzyme is a nucleotide regeneration enzyme.

239. The method according to embodiment 238, wherein the reagent regeneration enzyme achieves the conversion of UDP to UTP and is EM-3-1-1, EM-3-1-1-1, EM-3-1-1-2, EM-3-1-1-2-1, EM-3-1-1-2-2, EM-3-1-1-3, EM-3-1-1-4, EM-3-1-4-1-1, or EM-3-1-4-1-2.

240. The method according to embodiment 238, wherein the reagent regeneration enzyme achieves the conversion of GDP to GTP and is EM-3-1-2, EM-3-1-2-1, EM-3-1-2-1-1, EM-3-1-2-1-2, EM-3-1-2-2, EM-3-1-2-3, EM-3-1-4-1-1, or EM-3-1-4- 1-2.

241. The method according to embodiment 238, wherein the reagent regeneration enzyme achieves the conversion of CMP to CTP and is EM-EM-3-1-3, EM-3-1-3-1, EM-3-1-3-1-1, EM-3-1-3-1-2, EM-3-1-3-2, EM-3-1-3-2-1, EM-3-1-3-2-2, EM-3-1-3-3, EM-3-1-3-4, EM-3-1-3-5.

242. The method according to embodiment 238, wherein the reagent regeneration enzyme achieves the conversion of ADP to ATP and is EM-3-1-4-1, EM-3-1-4-1-1, EM-3-1-4-1-2, EM-3-1-4-2, or EM-3-1-4-3.

243. The method according to embodiment 237, wherein the reagent regeneration enzyme achieves sugar nucleotide regeneration.

244. The method according to embodiment 243, wherein the reagent regeneration enzyme us EM-3-2-, EM-3-2-1-1, or EM-3-2-1-1-1.

245. The method according to embodiment 237, wherein the reagent regeneration enzyme is a co-factor regeneration enzyme.

246. The method according to embodiment 245, wherein co-factor regeneration enzyme is EM-3-3-1, EM-3-3-2, EM-3-3-2-1, EM-3-3-2-2.

247. The method according to any one of embodiments 23-26, wherein the glycan synthesis enzyme is a functionalization enzyme that phosphorylates or sulfates glycans.

248. The method according to embodiment 247 wherein the phosphorylation enzyme is (EC 2.7.8.17) or GNPTG form Homo sapiens.

249. The method according to embodiment 247 wherein the sulfation enzyme is EM-4-2-1, EM-4-2-1-1, EM-4-2-1-2, EM-4-2-1-3, EM-4-2-1-4, EM-4-2-1-5, EM-4-2-1-6, EM-4-2-1-7, EM-4-2-1-8, EM-4-2-1-9, EM-4-2-1-10, EM-4-2-1-11, EM-4-2-1-12, EM-4-2-1-13, EM-4-2-1-14, EM-4-2-1-15, EM-4-2-2, EM-4-2-2-1, EM-4-2-2-2, EM-4-2-2-3, EM-4-2-3, EM-4-2-3-1, EM-4-2-3-2, EM-4-2-3-3, EM-4-2-3-4, EM-4-2-3- 5, EM-4-2-3-6, EM-4-2-3-7, EM-4-2-3-8, EM-4-2-3-9, EM-4-2-3-10, EM-4-2-3-11 EM-4-2-3-12, EM-4-2-3-13, EM-4-2-3-14, EM-4-2-4, EM-4-2-4-1, EM-4-2-4-2, EM-4-2-4-3, EM-4-2-4-4, or EM-4-2-4-5.

250. The method according to any one of embodiments 23-26, wherein the glycan synthesis enzyme is a support enzyme.

251. The method according to any one of embodiment 250, wherein the support enzyme is EM-5-1 or EM-5-1-1.

252. The method according to any one of embodiments 23-26, wherein the glycan synthesis enzyme is a sugar removal enzyme.

253. The method according to embodiment 252, wherein the sugar removal enzyme is a fucosidase,

254. The method according to embodiment 253, wherein the fucosidase is EM-6-1-1, EM-6-1-2, EM-6-1-3, EM-6-1-4, EM-6-1-4-1, or EM-6-1-4-2.

255. The method according to embodiment 252, wherein the sugar removal enzyme is a galactosidase,

256. The method according to embodiment 255, wherein the galactosidase is EM-6-2-1, EM-6-2-2, EM-6-2-2-1, EM-6-2-2-2, EM-6-2-2-3, or EM-6-2-2-4.

257. The method according to embodiment 252, wherein the sugar removal enzyme is a sialidase.

258. The method according to embodiment 257, wherein the sialidase is SM-1, SM-2, SM-3, SM-4, SM-5, SM-6, SM-7, SM-8, SM-9, SM-10, or SM-11.

259. The magnetic macroporous powder according to any of embodiments 23-258, wherein said magnetic particles have a size of between about 100-1000 μm.

260. The magnetic macroporous powder according to any of embodiments 23-258, wherein said magnetic particles have a size of between about 50-100 μm.

261. The magnetic macroporous powder according to any of embodiments 23-258, wherein said magnetic particles have a size of between about 10-50 μm.

262. The magnetic macroporous powder according to any of embodiments 23-258, wherein said magnetic particles have a size of between about 5-10 μm.

263. The magnetic macroporous powder according to any of embodiments 23-258, wherein said magnetic particles have a size of about 10 μm.

264. The magnetic macroporous according to any of embodiments 23-258, wherein said magnetic particles have a size of about 5 μm.

265. The magnetic macroporous powder according to any of embodiments 23-258, wherein said magnetic particles have a size of less than 5 μm.

266. The magnetic macroporous powder according to any of embodiments 23-258, wherein said magnetic particles have a size of greater than 100 μm.

267. The magnetic macroporous powder according to any of embodiments 23-258 having an average size of about 150 μm.

268. The magnetic macroporous powder according to any of embodiments 23-258 having an average size of about 75 μm.

269. The magnetic macroporous powder according to any of embodiments 23-258 having an average size of about 15 μm.

270. The magnetic macroporous powder according to any of embodiments 23-258, wherein said magnetic particles have a concentration of between 0 and 10% by weight.

271. The magnetic macroporous powder according to any of embodiments 23-258, wherein said magnetic particles have a concentration of 10 to 50% by weight.

272. The magnetic macroporous powder according to any of embodiments 23-258, wherein said magnetic particles have a concentration of 50 to 90% by weight.

273. The magnetic macroporous powder according to any of embodiments 23-258, wherein said thermoplastic polymer comprises a polymer selected from the group consisting of Polyvinyl alcohol (PVA), Acrylic (PMMA), Acrylonitrile butadiene styrene (ABS), Polyamide including Nylon 6 and Nylon 12, Polylactic acid (PLA), Polybenzimidazole (PBI), Polycarbonate (PC), Polyether sulfone (PES), Polyoxymethylene (POM), Polyetherether ketone (PEEK), Polyetherimide (PEI), Polyethylene (PE), Polyphenylene oxide (PEO), Polyphenylene sulfide (PPS), Polypropylene (PP), Polystyrene (PS), Polyvinyl chloride (PVC), polytetrafluoroethylene (PTFE), co-polyesters, and chemically functionalized derivatives thereof.

274. The magnetic macroporous powder according to any of embodiments 23-258, wherein said magnetic microparticles comprise a magnetic material selected from the group consisting of magnetite (Fe₃O₄), hematite (α-Fe₂O₃), maghemite (γ-Fe₂O₃), a spinel ferrite, lodestone, cobalt, nickel, rare earth, and magnetic composites.

275. The magnetic macroporous powder according to embodiment 274, wherein said rare earth is neodymium, gadolinium, dysprosium, samarium-cobalt, or neodymium-iron-boron.

276. The magnetic macroporous powder of embodiment 16, wherein said magnetic composite comprises a ceramic, ferrite, or alnico magnets.

277. The magnetic macroporous powder according to embodiment 273, wherein said thermoplastic polymer and said magnetic microparticles are chemically blended.

278. The magnetic macroporous powder according to embodiment 273, wherein said thermoplastic polymer and said magnetic microparticles are thermally blended.

279. The magnetic macroporous powder according to embodiment 273, wherein said thermoplastic polymer and said magnetic microparticles are physically blended.

280. The magnetic macroporous powder according to any of embodiments 23-258, comprising macropores having a size of between 0.5-200 μm.

281. The magnetic macroporous powder to any one of embodiments 23-258, further comprising cellulose fibers, cellulose nanofibers, glass fibers, or carbon fibers.

282. The magnetic macroporous powder of to any of embodiments 23-258, further comprising self-assembled mesoporous aggregates of magnetic nanoparticles and an enzyme magnetically immobilized within said mesopores or on their surface.

283. A shaped magnetic macroporous scaffold, comprising the magnetic macroporous powder according to any of embodiments 23-258, wherein said powder has been formed into said shape by three-dimensional (3D) printing.

284. The shaped magnetic macroporous scaffold of embodiment 283, wherein said shape is a cylinder, an orb, a bead, a strip, a capsule, a cube, a squared rod, a pyramid, a diamond, a lattice, or an irregular shape.

285. The shaped magnetic macroporous scaffold of either one of embodiments 283-284, further comprising self-assembled mesoporous aggregates of magnetic nanoparticles.

286. The shaped magnetic macroporous scaffold according to any one of embodiments 283-285, wherein said self-assembled mesoporous aggregates of magnetic nanoparticles further comprise one or more enzymes magnetically immobilized within said mesopores or on the surface of said magnetic nanoparticles.

287. The shaped magnetic macroporous scaffold of any embodiment 286, wherein said one or more enzymes are selected from the group consisting of hydrolases, hydroxylases, hydrogen peroxide producing enzymes (HPP), nitralases, hydratases, dehydrogenases, transaminases, ketoreductases (KREDS) ene reductases (EREDS), imine reductases (IREDS), catalases, dismutases, oxidases, dioxygenases, lipoxidases, oxidoreductases, peroxidases, laccases, synthetases, transferases, oxynitrilases, isomerases, gludosidases, kinases, lyases, sucrases, invertases, epimerases, and lipases.

288. The shaped magnetic macroporous scaffold of embodiment 287, wherein said self-assembled mesoporous aggregates of magnetic nanoparticles comprise microsomes, wherein a first enzyme requiring a diffusible cofactor having a first enzymatic activity is contained within said microsomes, wherein a second enzyme comprising a cofactor regeneration activity is magnetically-entrapped within said mesopores, wherein said cofactor is utilized in said first enzymatic activity; wherein said first and second enzymes function by converting a diffusible substrate into a diffusible product; and wherein said magnetic nanoparticles are magnetically associated with said magnetic macroporous scaffold.

289. The shaped magnetic macroporous scaffold of embodiment 288, wherein said self-assembled mesoporous aggregates of magnetic nanoparticles comprises a first enzyme requiring a diffusible cofactor having a first enzymatic activity; a second enzyme comprising a cofactor regeneration activity; wherein said cofactor is utilized in said first enzymatic activity; wherein said first and second enzymes are magnetically-entrapped within said mesopores formed by said aggregates of magnetic nanoparticles and said first and second enzymes function by converting a diffusible substrate into a diffusible product.

290. The shaped magnetic macroporous scaffold of embodiment 289, wherein said first enzyme is an oxidative enzyme.

291. The shaped magnetic macroporous scaffold of embodiment 290, wherein said oxidative enzyme is a Flavin-containing oxygenase; wherein said composition further comprises a third enzyme having a co-factor reductase activity that is co-located with said first enzyme.

292. The shaped magnetic macroporous scaffold of embodiment 291, wherein said oxidative enzyme is a P450 monooxygenase; wherein said composition further comprises a third enzyme having a co-factor reductase activity that is co-located with said first enzyme.

293. The shaped magnetic macroporous scaffold of embodiment 292, wherein said P450 monooxygenase and said third enzyme are comprised within a single protein.

294. The shaped magnetic macroporous scaffold of embodiment 35, wherein said single protein comprises a bifunctional cytochrome P450/NADPH—P450 reductase.

295. The shaped magnetic macroporous scaffold according to any one of embodiments 23-258, wherein said scaffold is formed in a shape suited for a particular biocatalytic process.

296. A method of making a shaped magnetic macroporous scaffold comprising the magnetic macroporous powder of any one of embodiments 23-258, comprising additively manufacturing (AM) said shaped magnetic macroporous scaffold using a three-dimensional (3D) printer, wherein said shape is taken from a 3D model.

297. The method of embodiment 296, wherein said 3D model is an electronic file.

298. The method of embodiment 297, wherein said electronic file is a computer-aided design (CAD) or a stereolithography (STL) file.

299. The method of any one of embodiments 296-298, wherein said AM is Fused Filament Fabrication (FFF) or Selective laser sintering (SLS).

300. The method of any one of embodiments 296-299, wherein said macropores are formed using a soluble agent selected from the group consisting of a salt, a sugar, or a small soluble polymer, and removing said soluble agent with a solvent.

301. A method of making a device for catalyzing an enzymatic reaction, comprising combining a shaped magnetic macroporous scaffold with self-assembled mesoporous aggregates of magnetic nanoparticles and an enzyme, wherein said enzyme is magnetically immobilized within said mesopores.

302. A method of catalyzing a reaction between a plurality of substrates, comprising exposing said magnetic macroporous powder according to any one of embodiments 23-258 to said substrates under conditions in which said enzyme catalyzes said reaction between said substrates.

303. A method of catalyzing a reaction between a plurality of substrates, comprising exposing said shaped magnetic macroporous scaffold according to any one of embodiments 23-258 to said substrates under conditions in which said enzyme catalyzes said reaction between said substrates.

304. The method of either one of embodiments 302 or 303, wherein said reaction is used in the manufacture of a pharmaceutical product.

305. The method of either one of embodiments 302 or 303, wherein said reaction is used in the manufacture of a medicament.

306. The method of either one of embodiments 302 or 303, wherein said reaction is used in the manufacture of a food product.

307. The method of either one of embodiments 302 or 303, wherein said reaction is used in the manufacture of a flavor.

308. The method of either one of embodiments 302 or 303, wherein said reaction is used in the manufacture of a fragrance.

309. The method of either one of embodiments 302 or 303, wherein said reaction is used in the manufacture of a sweetener.

310. The method of either one of embodiments 302 or 303, wherein said reaction is used in the manufacture of an agrochemical.

311. The method of either one of embodiments 302 or 303, wherein said reaction is used in the manufacture of an antimicrobial agent.

312. The method of either one of embodiments 302 or 303, wherein said reaction is used in the manufacture of a toxin.

313. The method of either one of embodiments 302 or 303, wherein said reaction is used in the manufacture of a detergent.

314. The method of either one of embodiments 302 or 303, wherein said reaction is used in the manufacture of a fuel product.

315. The method of either one of embodiments 302 or 303, wherein said reaction is used in the manufacture of a biochemical product.

316. The method of either one of embodiments 302 or 303, wherein said reaction is used in the manufacture of a paper product.

317. The method of either one of embodiments 302 or 303, wherein said reaction is used in the manufacture of a plastic product.

318. The method of either one of embodiments 302 or 303, wherein said reaction is used in a process for removing a contaminant from a solution.

319. The method of embodiment 318, wherein said solution is an aqueous solution, a solvent, or an oil.

320. The method of either one of embodiments 302 or 303, further comprising the step of removing said mesoporous aggregates and replacing them with a fresh preparation of mesoporous aggregates.

321. The method of either one of embodiments 302 or 303, wherein said method is carried out using flow cell and continuous manufacturing.

In order that the invention described herein may be more fully understood, the following examples are set forth. It should be understood that these examples are for illustrative purposes only and are not to be construed as limiting this invention in any manner.

### EXAMPLES

Generally, thermoplastic and magnetite scaffolds are made as disclosed in PCT/US19/53307, incorporated by reference herein in its entirety. Magnetic nanoparticles (125-2000 μg/ml, pH 9 water) and enzyme solutions (pH 9 water) are combined and the pH is adjusted to a predetermined, optimized value with or without 1-5 mM MgSO₄. The mixture is immediately delivered to flow cells, typically, but not necessarily, via a peristaltic pump (5-50 ml/min). They are allowed to recirculate over the course of 1-4 hours at room temperature. The flow cells are inserted into a glass encased column (e.g., a HiScale 16/20 column, GE Healthcare, 16 mm ID, 200 mm length) with two adjustable ends.

### ABBREVIATIONS

3′-SL: 3′Sialyllactose
2′-FL: 2′-fucosyllactose
LNFPI: Lacto-N-fucopentaose I
LNT: Lacto-N-tetraose
LNnT: Lacto-N-neotetraose
LNTII: Lacto-N-triose II
DSLNT: Disialyllacto-N-tetraose
UDP: Uridine 5′-diphosphate
UTP: Uridine 5′-triphosphate
ADP: Adenosine 5′-diphosphate
ATP: Adenosine 5′-triphosphate
CMP: Cytidine 5′-monosphate
CDP: Cytidine 5′-diphosphate
CTP: Cytidine 5′-triphosphate
GDP: Guanosine 5′-diphosphate
GTP: Guanosine 5′-triphosphate
Tris: Tris(hydroxymethyl)aminomethane
NiNTA: Nickel nitrilotriacetic acid

HPLC Analytical Methods Applicant uses an in-house Vanquish Duo HPLC system (Thermo Fisher Scientific) equipped with a charged aerosol detector (CAD) and a mixed-mode reverse phase Trinity P1 column (Thermo Fisher Scientific, 3×100 mm, DX071387). The percent molar conversion is calculated using the integrated starting material (SM) and product (P) peaks (Equation 2) while selecting the starting material that is stoichiometrically limiting. All peak areas are divided by their respective molar masses to convert from units of mass (CAD response is proportional to mass of analyte) to units of mol.

Equation 2. Determination of molar % conversion of limiting starting (SM) to product (P).

% molar conversion=( [symbol] Area [symbol] _P/(MW_P))/( [symbol] Area [symbol] _P/(MW_P)+ [symbol] Area [symbol] SM/(MW_SM))

All four reactions are run in a mobile phase of acetonitrile (solvent A) and ammonium formate (solvent B) using 2.5 μl injection volumes and a flow rate of 0.5 ml/min.

### Example 1: Cell Free Production of Simple Glycans

### Example 1a: Reagents and Materials

1a Reagents and materials: PmPpa, NmCSS, and PmST1, were expressed recombinantly in Escherichia coli (BL21 DE3) by the Bioexpression and Fermentation Facility (BFF) at the Complex Carbohydrate Research Center (CCRC) at the University of Georgia, Athens. The enzymes were purified from the soluble lysate by affinity chromatography (NiNTA) and the buffer was exchanged by dialyzing against 50 mM Tris pH 7.5. The enzymes were supplemented with 10% (w/w) glycerol and frozen at −80° C. for storage. The plasmids used for protein expression were produced by Genewiz by custom synthesis of the insert and splicing into a commercial pET28a vector (Novagen). The activity of enzymes and optimal ratio for the systems of enzymes were determined in free solution. The following chemical reagents were used in the reagent stream of flow cell assembly to synthesize HMOs: CTP (Cytidine-5′-triphosphate disodium salt, Alfa Aesar, AAJ62238ME); Lactose (D-Lactose monohydrate, Fisher Scientific, L5-500); Neu5Ac (N-Acetylneuraminic Acid Hydrate, TCI America, A06395G); Tris (TRIS, 1.0M buffer solution., pH 7.5, Alfa Aesar, J62993AP); MgCl₂ (Magnesium Chloride, Macron Fine Chemicals, 595804). All water was obtained from a BarnStead Nanopure water purifier (Thermo Scientific, 18.5 MOhm-cm).

Nanoparticle production: Magnetite (Fe₃O₄) nanoparticles (MNP) used for the immobilization of enzymes into BNCs were synthesized via coprecipitation of FeCl₂*4H₂O (Iron (II) chloride tetrahydrate, Fisher Scientific, AC205080010) and FeCl₃*6H₂O (Iron (III) chloride hexahydrate, Fisher Scientific, AC125030010) in degassed Milli-Q water at concentrations of 0.8 M and 1.6 M, respectively. NaOH (sodium hydroxide, VWR, MK7708-10) was prepared at a concentration of 2.8 M in degassed Milli-Q water and added dropwise to the iron salt solution. The reaction was performed at ambient temperature and pressure. The synthesized magnetite nanoparticles were purified by removing ions via decanting the water with the assistance of a permanent magnet, then adding fresh Milli-Q water back to the magnetite particles equal to the amount that was decanted off The decanting and addition of Milli-Q water was repeated a total of four times. The nanoparticles were dispersed in degassed pH 11 water in a polypropylene container solution while keeping the volume equal to the prior washing steps. The nanoparticle solution was finally degassed by sparging with nitrogen (N₂) gas for 10 min per 20 ml of solution. The concentration of the MNP solution was determined to be 32.1 mg/ml by weighing the dry weight of a 1 ml MNP slurry that was dried overnight in a vacuum oven.

### Example 1b: Batch Synthesis of 3′SL with Scaffolded BNCs

3′-SL was produced from lactose, Neu5Ac and CTP with system module 4 (SM4) using three elementary modules (EM1.4A, EM5.1A, EM2.3A; see FIG. 1 and FIG. 2). BNCs formed with 200 μg NmCSS, 1.0 mg PmPPa, and 1.25 mg PmST1 in a 25 ml volume of pH 9 water and 25 ml of a pH 9 nanoparticle solution (390 ug/ml). Scaffolded BNCs were formed by incrementally adding 125 mg of strontium ferrite scaffold (Powdertech, cat. no. S20) while mixing with an overhead stirred (500-1000 rpm). The pH was then incrementally lowered to pH 6.0 over the course of one hour. After an additional hour of incubation, the supernatant from 2 ml of the resulting mixture was removed and the remaining immobilized powder was washed twice with 25 mM Tris buffer. The immobilization yield was determined to be greater than 70% by Bradford assay. The remaining BNC functionalized powder was added to a 2 mL reaction mixture containing 5 mM lactose, 1.2 mM CTP, 1 mM Neu5Ac and 10 mM MgCl₂ in 25 mM Tris pH 8.8 and incubated at 37° C. for 2 hrs, which afforded greater than 75% conversion of 3′-SL from Neu5Ac by HPLC analysis. That yield corresponded to ˜90% of the yield compared to the free enzyme reaction (FIG. 4A).

The % conversion was analyzed by HPLC (System: Thermo Scientific Vanquish Duo; Column: Acclaim Trinity P1; CAD detection; mobile phase A1: acetonitrile; mobile phase B1: 50 mM ammonium formate pH 4.45; 1-5 min: 30% B1; 6-7 min 60% B1; 8-10 min 30% B1.

### Example 1c: In-Flow Synthesis of 3′SL with Scaffolded BNCs

1c In-flow synthesis of 3′SL with scaffolded BNCs: 3′-SL was produced from lactose, NeuSAc and CTP with system module 4 (SM4) using three elementary modules (EM1.4A, EM5.1A, EM2.3A; see FIG. 1 and FIG. 2). BNCs formed with 800 μg NmCSS, 2.0 mg PmPPa, and 5.0 mg PmST1 in a 100 ml volume of pH 9 water and 25 ml of a pH 9 nanoparticle solution (390 ug/ml). Scaffolded BNCs were formed by incrementally adding 1 g of strontium ferrite scaffold (Powdertech, cat. no. S20) while mixing with an overhead stirred (500-1000 rpm). The pH was then incrementally lowered to pH 6.0 over the course of an hour. After an additional one hour of incubation, the supernatant was removed, and the scaffolded BNCs were washed with water. The immobilization yield was determined to be 96% by Bradford assay. The BNCs functionalized powder was transferred and packed into a column (length 2″, ID 1/16″). A syringe pump (New Era Pump Systems) then delivered the reagent stream (8 ml: 5 mM lactose, 6 mM CTP, 5 mM NeuSAc and 10 mM MgCl₂ in 25 mM Tris pH 8.8 at 37° C.) at 0.5 ml/hr into module SM4 and afforded 3′-SL at 29.68% conversion overnight (16 hrs) from lactose based on HPLC analysis which corresponded to >60% of the yield of the free enzyme reaction. Production of 3′SL in flow is shown in the HPLC chromatogram (FIG. 4B).

The % conversion as analyzed by HPLC (System: Thermo Scientific Vanquish Duo; Column: Acclaim Trinity P1; CAD detection; mobile phase A1: acetonitrile; mobile phase B1: 50 mM ammonium formate pH 4.45; 1-5 min: 30% B1; 6-7 min 60% B1; 8-10 min 30% B1.

### Example 2: Cell-Free In-Flow Production of Complex Glycans

### Example 2a: Reagents and Materials

Mammalian enzymes B3GNT2 and ST6GalNAc5 is purchased from Glyco Expression Technologies Inc. (Athens, Georgia). BbhI, Cv133GalT, PmPpa, SuS, exo-Sialidase, NmCSS, StGalE, NmLgtB, BfFKP, PmST1, Pd26ST, WbgL, Te2FT, Bf13FT, CMPK and NDPK are expressed recombinantly in Escherichia coli (BL21 DE3) by the Bioexpression and Fermentation Facility (BFF) at the Complex Carbohydrate Research Center (CCRC) at the University of Georgia, Athens or Genway Biotech Inc. (San Diego, CA). The enzymes are purified from the soluble lysate by affinity chromatography (NiNTA) and the buffer is exchanged by dialyzing against 50 mM Tris pH 7.5. The enzymes are supplemented with 10% (w/w) glycerol and frozen at −80° C. for storage. The plasmids used for protein expression are produced by Genewiz by custom synthesis of the insert and splicing into a commercial pET28a vector (Novagen).

The following chemical reagents are used in the reagent stream of flow cell assembly to synthesize HMOs: GlcNAc-oxazoline (Carbosynth, MM10955), ATP (Adenosine-5′-triphosphate disodium salt hydrate, Alfa Aesar, AAJ6112506); GTP (Guanosine-5′-triphosphate disodium salt, Alfa Aesar, AAJ61414MD); CTP (Cytidine-5′-triphosphate disodium salt, Alfa Aesar, AAJ62238ME); UTP (Uridine 5′-triphosphate, trisodium salt, hydrate, ACROS Organics, AC226310010); Glc-UDP (UDP-D-glucose disodium salt, Biosynth Carbosynth, MU08960); Lactose (D-Lactose monohydrate, Fisher Scientific, L5-500); Galactose (D(+)-Galactose, Acros Organics, 150611000); GlcNAc (N-Acetyl-D-glucosamine, Sigma Aldrich, A8625); Fucose (L-fucose, Biosynth Carbosynth, MF06710); Sucrose (Sucrose, Biosynth Carbosynth, OS02339); NeuSAc (N-Acetylneuraminic Acid Hydrate, TCI America, A06395G); 3′,6′-di-O-β-GlcNAc-lactose (Chemlily LLC, HMO1); Tris (TRIS, 1.0M buffer soln., pH 7.5, Alfa Aesar, J62993AP); MnSO₄ (manganese (II) sulfate monohydrate (Sigma Aldrich, M7634); MgCl₂ (Magnesium Chloride, Macron Fine Chemicals, 595804). All water was obtained from a BarnStead Nanopure water purifier (Thermo Scientific, 18.5 MOhm-cm).

### Example 2b: One-Pot Immobilization of Enzymes in Scaffolded BNCs

Scaffolded BNCs were formed by mixing enzyme or enzyme systems in a 25 ml volume of pH 9 water and 25 ml of a pH 9 magnetic nanoparticle solution (MNP final concentration depends on the enzyme system and ranges between 100-2000 ug/ml) by incrementally adding 250 to 1000 mg of strontium ferrite scaffold (Powdertech, cat. no. S20) while mixing with an overhead stirred (500-1000 rpm). The pH was then incrementally lowered to pH 6.0 over the course of an hour with the possibility of incremental addition of MgCl₂ to a final concentration of 0.2-2 mM. After an additional one hour of incubation, the supernatant was removed, the immobilized material was washed with water and the immobilization yield is determined using the Bradford assay. The scaffolded BNCs were transferred and packed into a column (length 2″, ID 1/16″). A syringe pump (New Era Pump Systems) then delivered the reagent stream at 0.25-2 ml/hr into the system module to afford the target glycan. The % conversion from starting material was determined by HPLC analysis and compared to the % conversion of the corresponding free enzyme reaction.

### Example 2c: Production of LNTII, LNT, LNnT, LNFPI, LNFPII, LNFPIII, LSTa, LSTb, LSTc, LSTd, DSLNT, and 2′-FL with BNCs

The following examples illustrate the step-by-step syntheses of LNTII, LNT, LNnT, LNFPI, LNFPII, LNFPIII, LSTa, LSTb, LSTd and DSLNT as shown in FIG. 2 using elementary modules of BNCs that are combined into sets of system modules as shown in FIG. 1. The target glycans may be synthesized step-by-step by sequentially combining below examples. Alternatively, the target glycans may be synthesized from any advanced glycan building block.

### A. Production of LNTII.

LNTII is produced from lactose, and N-acetyl glucosamine-oxazoline (glcNAc-Oxa) with system module 6 (SM6) using one elementary module (EM2.2A; see FIG. 1 and FIG. 2). BNCs (15 mg BbhI) are immobilized in a 25 ml volume on 250-1000 mg Strontium ferrite scaffold (Powdertech, cat. no. S20) as described in Section 2b, and the BNC functionalized powder is packed into a flow cell (length 2″, ID 1/16″). A syringe pump (New Era Pump Systems) delivers the reagent stream (7.2 ml) at 0.5 ml/hr into module SM6. The reagent stream consists of 60 mM lactose and 60 mM GlcNAc-oxa in 50 mM sodium phosphate buffer pH 7.5 at 37° C.

### B. Production of LNnT

LNnT is produced from LNTII, UDP-glucose (UDP-glc), and sucrose with system module 3 (SM3) consisting of three elementary modules (EM1.3A, EM2.3H, EM3.2A; see FIG. 1 and FIG. 2). Enzymes (40 μg SuS, 180 μg NmLgtB and 90 μg StGalE) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM3. The reagent stream consists of 25 mM LNTII, 5 mM UDP-glc, 50 mM sucrose and 10 mM MgCl₂ in 50 mM Tris pH 7.5 at 37° C.

### C. Production of LNT

LNT is produced from LNTII, UDP-glucose (UDP-glc), and sucrose with system module 3 (SM3) using three elementary modules (EM1.3A, EM2.3G, EM3.2A; see FIG. 1 and FIG. 2). BNCs (40 μg SuS, 90 μg StGalE and 180 μg Cvβ3GalT) are immobilized in a 25 ml volume on 250-1000 mg of strontium ferrite scaffold (Powdertech, cat. no. S20) as described in Section 2b, and the BNC functionalized powder is packed into a flow cell (length 2″, ID 1/16″). A syringe pump (New Era Pump Systems) delivers the reagent stream (7.2 ml) at 0.5 ml/hr into module SM3. The reagent stream consists of 25 mM LNTII, 5 mM UDP-Glc, 50 mM sucrose and 10 mM MgCl₂ in 50 mM Tris pH 7.5 at 37° C.

### D. Production of 2′-FL

2′-FL is produced from lactose, fucose, ATP and GTP with system module 1 (SM1) using four elementary modules (EM1.1A, EM2.3B, EM3.1A, EM5.1A; see FIG. 1 and FIG. 2). BNCs (1.44 mg FKP, 144 μg PmPPa, 2.16 mg WbgL and 144 μg NDPK) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM1. The reagent stream consists of 25 mM lactose, 125 mM ATP, 30 mM fucose, 20 mM GTP and 10 mM MgCl₂ in 50 mM Tris pH 8.0 at 37° C.

### E. Production of LNFPII

LNFPII is produced from LNT, fucose, ATP and GTP with system module 1 (SM1) using four elementary modules (EM1.1A, EM2.3C, EM3.1A, EM5.1A; see FIG. 1 and FIG. 2). BNCs (1.44 mg FKP, 144 μg PmPpa, 2.16 mg Bf13FT and 144 μg NDPK) are immobilized in a 25 ml volume on 250-1000 mg of strontium ferrite scaffold (Powdertech, cat. no. S20) as described in Section 2b, and the BNC functionalized is packed into a column (length 2″, ID 1/16″). A syringe pump (New Era Pump Systems) delivers the reagent stream (7.2 ml) at 0.5 ml/hr into module SM1. The reagent stream consists of 25 mM LNT, 125 mM ATP, 30 mM fucose, 20 mM GTP and 10 mM MgCl₂ in 50 mM Tris pH 8.0 at 37° C.

### F. Production of LNFPI

LNFPI is produced from LNT, fucose, ATP and GTP with system module 1 (SM1) using four elementary modules (EM1.1A, EM2.3A, EM3.1A, EM5.1A; see FIG. 1 and FIG. 2). BNCs (1.44 mg FKP, 144 μg PmPpa, 2.16 mg Te2FT and 144 μg NDPK) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM1. The reagent stream consists of 25 mM LNT, 125 mM ATP, 30 mM fucose, 20 mM GTP and 10 mM MgCl₂ in 50 mM Tris pH 8.0 at 37° C.

### G. Production of LSTc

LSTc is produced from LNnT, NeuSAc, ATP and CTP with system module 4 (SM4) using four elementary modules (EM1.4A, EM2.3E, EM3.1B, EM5.1A; see FIG. 1 and FIG. 2). BNCs (28.8 ug NmCSS, 144 μg PmPPa, 144 μg CMPK, 144 μg NDPK and 180 μg ST6GAL1) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM4. The reagent stream consists of 25 mM LNnT, 10 mM CTP, 125 mM ATP, 30 mM NeuSAc and 10 mM MgCl₂ in 50 mM Tris pH 8.8 at 37° C.

### H. Production of LNFPIII

LNFPIII is produced from LNnT, fucose, ATP and GTP with system module 1 (SM1) consisting of four elementary modules (EM1.1A, EM2.3C, EM3.1A, EM5.1A; see FIG. 1 and FIG. 2). BNCs (1.44 mg FKP, 144 μg PmPpa, 2.16 mg Bf13FT and 144 μg NDPK) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM1. The reagent stream consists of 25 mM LNnT, 125 mM ATP, 30 mM fucose, 20 mM GTP and 10 mM MgCl₂ in 50 mM Tris pH 8.0 at 37° C.

### I. Production of LSTd

LSTd is produced from LNnT, NeuSAc, ATP and CTP with system module 4 (SM4) using four elementary modules (EM1.4A, EM2.3D, EM3.1B, EM5.1A; see FIG. 1 and FIG. 2). BNCs (28.8 μg NmCSS, 144 μg PmPpa, 144 μg CMPK, 144 μg NDPK and 180 μg PmST1) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM4. The reagent stream consists of 25 mM LNnT, 10 mM CTP, 125 mM ATP, 30 mM NeuSAc and 10 mM MgCl₂ in 50 mM Tris pH 8.8 at 37° C.

### J. Production of LSTa

LSTa is produced from LNT, NeuSAc, ATP and CTP with system module 4 (SM4) using four elementary modules (EM1.4A, EM2.3D, EM3.1B, EM5.1A; see FIG. 1 and FIG. 2). BNCs (28.8 μg NmCSS, 144 μg PmPpa, 144 μg CMPK, 144 μg NDPK and 180 μg PmST1) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM4. The reagent stream consists of 25 mM LNT, 10 mM CTP, 125 mM ATP, 30 mM NeuSAc and 10 mM MgCl₂ in 50 mM Tris pH 8.8 at 37° C.

### K. Production of DSLNT

DSLNT is produced from LSTa, NeuSAc, ATP and CTP with system module 4 (SM4) using four elementary modules (EM1.4A, EM2.3F, EM3.1B, EM5.1A; see FIG. 1 and FIG. 2). BNCs (28.8 μg NmCSS, 144 μg PmPpa, 144 μg CMPK, 144 μg NDPK and 180 μg ST6GalNAC5) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM4. The reagent stream consists of 25 mM LSTa, 10 mM CTP, 125 mM ATP, 30 mM NeuSAc and 10 mM MgCl₂ in 50 mM Tris pH 8.8 at 37° C.

### L. Production LSTb

LSTb is produced from DSLNT with system module 11 (SM11) consisting of one elementary module (EM6.3A; see FIG. 1 and FIG. 2). BNCs (2.5 U exo-sialidase) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM11. The reagent stream consists of 25 mM DSLNT and 10 mM MgCl₂ in 50 mM Tris pH 7.5 at 37° C.

### Example 2d: Production of LNnH, 3′″₃,3′″₆-di-O-α-Sia-LNnH and 3′″₃,3′″₆-di-O-α-Sia-(3″₃, 3″₆-di-O-α-Fuc)-LNnH

The following examples illustrate the step-by-step syntheses of LNnH, 3′″₃,3′″₆-di-O-α-Sia-LNnH and 3′″₃,3′″₆-di-O-α-Sia-(3″₃,3″₆-di-O-α-Fuc)-LNnH as shown in FIG. 3 using elementary modules that are combined into sets of system modules as shown in FIG. 1. The target glycans may be synthesized step-by-step by sequentially combining below examples. Alternatively, the target glycans may be synthesized from any advanced glycan building block.

### A. Production of LNnH

LNnH is produced from 3′,6′-di-O-β-GlcNAc-lactose, UDP-glucose (UDP-glc), and sucrose with system module 3 (SM3) using three elementary modules (EM1.3A, EM2.3H, EM3.2A; see FIGS. 1&3). BNCs (40 μg SuS, 90 μg StGalE and 180 μg NmLgtB) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM3. The reagent stream consists of 25 mM 3′,6′-di-O-β-GlcNAc-lactose, 10 mM UDP-glc, 100 mM sucrose and 10 mM MgCl₂ in 50 mM Tris pH 7.5 at 37° C.

B. Production of 3′″₃,3′″₆-di-O-α-Sia-LNnH (DSLNnH)

3′″₃,3′″₆-di-O-α-Sia-LNnH is produced from LNnH, Neu5Ac, ATP and CTP with system module 4 (SM4) consisting of four elementary modules (EM1.4A, EM2.3D, EM3.1B, EM5.1A; see FIGS. 1&3). BNCs (57.6 μg NmCSS, 288 μg PmPpa, 288 μg CMPK, 288 μg NDPK and 360 μg PmST1) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM4. The reagent stream consists of 25 mM LNnH, 20 mM CTP, 250 mM ATP, 60 mM Neu5Ac and 10 mM MgCl₂ in 50 mM Tris pH 8.8 at 37° C.

C. Production of 3′″₃,3′″₆-di-O-α-Sia-(3″3,3″6-di-O-α-Fuc)-LNnH (DSDFLNNH)

3′″3,3′″₆-di-O-α-Sia-(3″₃,3″₆-di-O-α-Fuc)-LNnH is produced from 3′″₃,3′″₆-di-O-α-Sia-LNnH, fucose, ATP and GTP with system module 1 (SM1) using four elementary modules (EM1.1A, EM2.3C, EM3.1A, EM5.1A; see FIGS. 1&3). BNCs (1.44 mg FKP, 288 μg PmPpa, 4.32 mg Bf13FT and 288 μg NDPK) are immobilized in a 25 ml volume on 250-1000 mg of strontium ferrite scaffold (Powdertech, cat. no. S20) as described in Section 2b, and the BNC functionalized powder is packed into a column (length 2″, ID 1/16″). A syringe pump (New Era Pump Systems) delivers the reagent stream (7.2 ml) at 0.5 ml/hr into module SM1. The reagent stream consists of 25 mM 3′″₃,3′″₆-di-O-α-Sia-LNnH, 250 mM ATP, 60 mM fucose, 40 mM GTP and 10 mM MgCl₂ in 50 mM Tris pH 8.0 at 37° C.

### Example 3: Cell-free Production Of Glycans

### Example 3a: Reagents and Materials

3a Reagents and materials: Enzymes PmST1, NmCSS, HmFucT, Te2FT, BfFKP, PmPpa, NDPK, CMPK and BbhI (Table 2) were cloned and produced as described in “Enzyme Production and Cloning”. Briefly, all enzymes were recombinantly expressed in Escherichia coli (BL21 DE3) either by Zymtronix or the Bioexpression and Fermentation Facility (BFF) at the Complex Carbohydrate Research Center (CCRC) at the University of Georgia, Athens. The enzymes were purified from the soluble lysate by affinity chromatography (NiNTA) and buffer exchanged either by dialysis or size exclusion chromatography against 50 mM Tris pH 7.5-8.0, 10% glycerol. The enzymes were frozen at −80° C. for storage. The plasmids used for protein expression were produced by Genewiz by custom synthesis of the insert and splicing into a commercial pET28a vector (Novagen). The activity of enzymes and optimal ratio for the systems of enzymes were determined in free solution. The following chemical reagents were used in the reagent stream of flow cell assembly to synthesize HMOs: D-lactose monohydrate (Sigma Aldrich, 61345); Fucose (L-fucose, Biosynth Carbosynth, MF06710); N-Acetylneuraminic Acid Hydrate (TCI America, A06395G); Lacto-N-tetraose (Elicityl, GLY010); ATP (Adenosine-5′-triphosphate disodium salt hydrate, Carbosynth, NA00135); GTP (Guanosine-5′-triphosphate disodium salt, Carbosynth, NG01208); CTP (Cytidine-5′-triphosphate disodium salt, Sigma Aldrich, C1506); GlcNAc (N-Acetyl-D-glucosamine, TCI, A0092); Triethylamine (Alfa Aesar, A12646); DMC (2-Chloro-4,5-dihydro-1,3-dimethyl-1H-imidazolium chloride, TCI, C14085G); Tris (TRIS, 1.0M buffer soln., pH 7.5, Alfa Aesar, J62993AP); MgCl₂ (Magnesium Chloride, Macron Fine Chemicals, 595804). All water was obtained from a BarnStead Nanopure water purifier (Thermo Scientific, 18.5 MOhm-cm).

Nanoparticle production: Magnetite (Fe₃O₄) nanoparticles (MNP) used for the immobilization of enzymes into BNCs are synthesized via continuous coprecipitation. FeCl2*4H2O (Iron (II) chloride tetrahydrate, Fisher Scientific, AC205080010) and FeCl3*6H2O (Iron (III) chloride hexahydrate, Fisher Scientific, AC125030010) are combined into degassed Milli-Q water at concentrations of 0.8 M and 1.6 M, respectively. NaOH (sodium hydroxide, VWR, MK7708-10) is prepared at a concentration of 2.8 M in degassed Milli-Q water. The iron salt solution is pumped into an agitated reactor at a rate of 10 mL/min, while the NaOH solution is pumped into the reactor at a rate of 25 mL/min. Mixing is done with a 1″ diameter pitched blade turbine at 300 RPM. The reactor level is kept constant at 35 mL with an outlet pump set to 35 mL/min. The reaction occurs at ambient temperature.

The synthesized magnetite particles are purified by removing ions via decanting the water with the assistance of a magnet, then adding fresh Milli-Q water back to the magnetite particles equal to the amount that was decanted off. The decanting and addition of Milli-Q water is repeated a total of four times. The concentration of the MNP solution was determined by weighing the dry weight of a 1 ml MNP slurry that was dried overnight in a vacuum oven.

### Enzyme Immobilization Method and Protocol:

The enzyme immobilization matrix (scaffold) consists of magnetite nanoparticle (MNP) covered strontium ferrite (SFE) particles (powders). The SFE powders are commercially available upon request from Powdertech International as a spherical particle with a tight size distribution of an average particle diameter of either 20 μm (S20) or 40 μm (S40W; wrinkled). The coating was achieved by addition of a nanoparticle solution at pH 10.0 to an aqueous scaffold solution at pH 10. The pH was slowly reduced to around pH 7.5 at which point the nanoparticles have fully coated the SFE scaffold (FIG. 9A). SEM (scanning electron microscopy) images of the MNP coated SFE scaffold are shown in FIGS. 9B and 9C. Enzymes are then added and immobilized on the surface of the nanoparticle coat. The specific amounts of scaffold and MNP employed are listed for each example. A representative protocol is as follows: 2.0 g of SFE scaffold (S20 or S40W) was suspended in 50 mL of pH 10 water and was agitated with an overhead stirrer (900 rpm) at ambient temperature. A 50 mL MNP solution (2×, 1.20 mg/mL) at pH 10 was added at a rate of 5 mL/min. As the pH decreased to ˜7.5, the MNPs aggregated onto the scaffold as evidenced by the clearing of the solution and as quantified by the reduction in Abs400 nm—comparable to that of water. The MNP-modified scaffold was washed with 100 ml of neutral water. In a second step, 10.0 ml of water was removed and replaced at a flowrate of 1 mL/min with 10.0 mL of enzyme solution (10×) at pH 7.5 for the co-immobilization. The pH was slowly reduced to 6.0. The solution (100 mL) was stirred for an additional 30 minutes at which point the enzyme immobilization yield was determined using the Bradford method (Reference: Bradford MM. A rapid and sensitive method for the quantitation of microgram quantities of protein utilizing the principle of protein-dye binding. Analytical biochemistry 1976; 72:248-54).

### Example 3b: Batch Synthesis of 3′SL with Scaffolded BNCs

3′-SL was produced from lactose, Neu5Ac, ATP and CTP with system module 4 (SM4) using four elementary modules (EM1.4A, EM5.1A, EM2.3A; see FIGS. 1&2) with NmCSS, PmPpa, CMPK, NDPK and PmST1 immobilized on S40W in a batch reactor.

Scaffolded BNC preparation: 2.0 g of S40W was scaffolded with 40 mg of magnetite nanoparticles (40 mg; 2% w/w) as described (“Enzyme Immobilization Method and Protocol”) onto which 1.6 mg NmCSS, 1.0 mg PmPpa, 4.0 mg CMPK, 4.0 mg NDPK and 2.5 mg PmST1 were added in 4.1 ml of 20 mM Tris pH 7.5. Bradford analysis showed that 98.2% of enzymes were immobilized. For the 1X immobilized enzyme reaction (Immob. Enzyme 1X) 1.8 ml of BNC suspension was transferred to 2.0 ml microtubes, the aqueous supernatant was removed, and the BNC was washed with two 1.0 ml volumes of water before adding the reaction feed. For the 5X immobilized enzyme reaction (Immob. Enzyme 5X) 9.0 ml of BNC suspension was transferred to a 15 ml Falcon tube, the aqueous supernatant was removed, and the BNC was washed with two 1.0 ml volumes of water while transferring the BNC suspension to a 2.0 ml microtube. 1.8 ml of reaction feed was added to both “Immob. Enzyme 1X” and “Immob. Enzyme 5X” containing tubes.

Preparation of reaction feed stock. The feedstock comprising 5 mM lactose, 7.5 mM Neu5Ac, 6 mM CTP and 15 mM ATP in 50 mM Tris pH 9.0 with 50 mM MgCl₂ was prepared by dissolving 21.6 mg of lactose, 31.0 mg of Neu5Ac, 105.8 mg of ATP and 40.0 mg of CTP in 9.0 ml of water followed by addition of 0.6 ml of Tris buffer (1M, pH 9.0) and 0.6 ml of 1 M MgCl₂. After the pH was adjusted to 9.0 with the addition of 1M NaOH, the final volume of feed stock was adjusted to 12 ml with additional water.

Batch reaction: To each 2.0 ml microtube was added 1.8 ml of reaction feed. The reaction was incubated at 37° C. and rotated for 1.0 hr. 10 μl fractions were collected at 10, 20, 30, 45 and 60 minutes and analyzed by HPLC analysis. HPLC system: Thermo Scientific Vanquish Duo; Column: Acclaim Trinity P1; CAD detection. Solvent A: Acetonitrile; solvent B: 50 mM ammonium formate pH 4.5. 0-1 min (30% B); 1-5 min (30-60% B gradient), 5-8 min (60% B), 8-9 min (60-30% B gradient), and 9-10 min (30% B).

Full (100%) conversion was achieved after 20 min with the immobilized 1X enzyme system with negligible sialidase activity while 99% conversion was achieved after 10 minutes with the immobilized 5X enzyme system (FIG. 4C).

### Example 3c: In-Flow Synthesis of 3′SL with Scaffolded BNCs

3′-SL was produced with system module 4 (SM4) using three elementary modules (EM1.4A, EM5.1A, EM2.3D; see FIGS. 1&2) from lactose, Neu5Ac and CTP in a continuous flow reactor (in-flow) with immobilized NmCSS, PmPpa and PmST1.

Scaffolded BNC preparation: 1 g of S20 was scaffolded with magnetite nanoparticles (10 mg; 1% w/w) as described (“Enzyme Immobilization Method and Protocol”) onto which enzymes NmCSS (0.8 mg), PmPpa (2.0 mg) and PmST1(5.0 mg) were added. The immobilization yield was determined to be 96% by Bradford assay.

Preparation of reaction feed stock: The feedstock was prepared with 5 mM lactose, 5 mM Neu5Ac and 6 mM CTP in 25 mM Tris pH 8.8 with 10 mM MgCl₂ 8.0 ml of 25 mM Tris buffer (pH 8.8).

Flow reaction: The feed stock solution was loaded to the column (length 2″, ID 1/16″) using a syringe pump (New Era Pump Systems) at a flow rate of 0.5 ml/hr at 37C, and 0.5 ml fractions were collected and analyzed by HPLC analysis. The % conversion was analyzed by HPLC (System: Thermo Scientific Vanquish Duo; Column: Acclaim Trinity P1; CAD detection; mobile phase A1: acetonitrile; mobile phase B1: 50 mM ammonium formate pH 4.45; 1-5 min: 30% B1; 6-7 min 60% B1; 8-10 min 30% B1.

3′-SL was produced with a 29.68% conversion overnight (16 hrs) from lactose which corresponded to >60% of the yield of the free enzyme reaction. Production of 3′SL in flow is shown in the HPLC chromatogram (FIG. 4D).

### Example 3c-A. In-Flow Synthesis of LNTII

LNTII was produced with system module 6 (SM6) using one elementary module (EM2.2A; see FIGS. 1D&2A) from lactose and the unpurified reaction product comprising GlcNAc oxazoline (GlcNAc-oxa) in continuous flow reactor (in-flow) with immobilized Bbh1.

Preparation of reaction feed stock: GlcNAc-oxa was produced by dissolving GlcNAc (884 mg, 4.0 mmol) and triethylamine (5.0 mL, 36.0 mmol) in water (8.0 ml) and cooling the solution on ice to 0° C. DMC (2032 mg, 12.0 mmol) was added to the solution and the mixture was stirred for 0.5 h at 0° C. Then, 2.0 ml of tris buffer (1.0 M, pH 7.5) and 1584 mg of lactose (4.40 mmol) were added to the reaction mixture. The pH of reaction mixture was adjusted to 7.5 by adding HCl (5 M) solution slowly at 0° C. The final volume of the feed stock was adjusted to 20 ml. The final concentration of GlcNAc-oxa, lactose and Tris were 200 mM, 220 mM and 100 mM respectively with a final pH of 7.5. (Reference: J. Org. Chem. 2009, 74, 2210-2212).

BNC preparation: 2.0 g of S40W was scaffolded with 40 mg of magnetite nanoparticles (40 mg; 2% w/w) as described (“Enzyme Immobilization Method and Protocol”) onto which enzyme (Bbh1) stock solution (4.0 mg Bbh1) was added. Bradford analysis showed that 100% of Bbh1 was immobilized. The Bbh1 immobilized scaffold (BNC) was packed into a column (ID 0.25″×length 2.0″).

Flow reaction: The feed stock solution was loaded to the column using a syringe pump (New Era Pump Systems) with a flow rate of 8.0 ml/hr and 1.5 ml fractions were collected and analyzed by HPLC (System: Thermo Scientific Vanquish Duo; Column: Acclaim Trinity P1; CAD detection.; Solvent A: Acetonitrile; solvent B: 20 mM ammonium formate pH 4.45. 0-2 min: 22% of B; 2-4 min: 22-35% of B; 4-5.5 min: 35% of B; 5.5-8 min: 35-70% of B; 8-10.5 min 70% of B; 10.5-13.5 min: 70-22% of B; 13.5-15 min: 22% of B. LNTII was produced over the course of 2.25 hrs. at a constant concentration of 100 mM that corresponds to an average conversion of 46% (FIG. 5).

### Example 3c-B. Production of 2′-FL (in-Flow) and Model System

2′-FL was produced from lactose, fucose, ATP and GTP with system module 1 (SM1) using four elementary modules (EM1.1A, EM2.3B, EM3.1A, EM5.1A; see FIG. 1 and FIG. 2) in a continuous flow reactor (in-flow) with immobilized BfFKP, PmPpa, HmFucT, and NDPK.

BNC preparation: 34.6 mg BfFKP, 2.4 mg PmPpa, 23.0 mg HmFucT and 4.6 mg NDPK were prepared in 115 ml of 20 mM Tris pH 7.5 and added to 12.8 ml (0.20 ml resin per mg enzyme) of NiNTA resin (Nickel NTA Agarose Beads, GoldBio cat. no. H-350-25). The mixture was rotated in a tube at 4° C. for one hour. The immobilization yield was determined by Bradford to be 85%. The resin was packed into tubing (length 14.0″×ID 0.25″) and washed with two bed volumes of 200 mM Tris pH 7.5 with 50 mM MgCl₂.

Preparation of reaction feed stock: The feedstock comprising 25 mM lactose, 30 mM L-fucose, 30 mM GTP and 75 mM ATP in 200 mM Tris pH 7.5 with 50 mM MgCl₂ was prepared by dissolving 900 mg of lactose, 493 mg of L-fucose, 4.41 g of ATP and 1.85 g of GTP in 50 ml of water followed by addition of 20 ml of Tris buffer (1M, pH 7.5) and 5.0 ml of 1 M MgCl₂. After the pH was adjusted to 7.5 with the addition of 1M NaOH, the final volume of feed stock was adjusted to 100 ml with additional water.

Flow reaction: The feed stock solution was loaded to the column using a syringe pump (New Era Pump Systems) at a flow rate of 1.0 ml/hr, and 2.0 ml fractions were collected and analyzed by HPLC analysis. HPLC system: Thermo Scientific Vanquish Duo; Column: Acclaim Trinity P1; CAD detection. Solvent A: Acetonitrile; solvent B: 20 mM ammonium formate pH 4.5. 0-5 min (22% B); 5-8 min (22-70% B gradient); 8-12 min (70% B); 12-14 min (70-22% B gradient); 14-15 min (22% B).

2′-FL was produced over the course of 72 hrs at an average conversion of lactose to 2′-FL of 45% which is equivalent to an average 2′-FL concentration of 5 mM (FIG. 6) resulting in a productivity of 2.5 mg 2′FL per hour.

### Example 3c-C. Production of LNFPI (in Batch)

LNFPI was produced from LNT, fucose, ATP and GTP with system module 1 (SM1) using four elementary modules (EM1.1A, EM2.3A, EM3.1A, EM5.1A; see FIGS. 1&2) with BfFKP, PmPpa, NDPK and Te2FT immobilized on S20 in a batch reactor.

BNC preparation: 1.8 g of S20 was scaffolded with 54 mg of magnetite nanoparticles (54 mg; 3% w/w) as described (“Enzyme Immobilization Method and Protocol”) onto which 7.5 mg BfFKP, 0.5 mg PmPpa, 0.5 mg NDPK and 7.5 mg Te2FT were added in 11.5 ml of 20 mM Tris pH 7.5. Bradford analysis showed that 97.1% of enzymes were immobilized. For the 1X immobilized enzyme reaction (Immob. Enzyme 1X) 1.8 ml of BNC suspension was transferred to 2.0 ml microtubes, the aqueous supernatant was removed, and the BNC was washed with two 1.0 ml volumes of water before adding the reaction feed. For the 5X immobilized enzyme reaction (Immob. Enzyme 5X) 9.0 ml of BNC suspension was transferred to a 15 ml Falcon tube, the aqueous supernatant was removed, and the BNC was washed with two 1.0 ml volumes of water while transferring the BNC suspension to a 2.0 ml microtube. 1.8 ml of reaction feed was added to both “Immob. Enzyme 1X” and “Immob. Enzyme 5X” containing tubes.

Preparation of reaction feed stock: The feedstock comprising 1.0 mM LNT, 1.5 mM L-Fucose, 1.2 mM GTP and 4 mM ATP in 25 mM Tris pH 8.0 with 20 mM MgCl₂ was prepared by dissolving 9.9 mg of LNT, 3.5 mg of L-Fucose, 10.3 mg of GDP, and 32.9 mg of ATP in 4.0 ml of water followed by addition of 0.35 ml of Tris buffer (1M, pH 8.0) and 0.28 ml of 1 M MgCl₂. After the pH was adjusted to 8.0 with the addition of 1M NaOH, the final volume of feed stock was adjusted to 14 ml with additional water.

Batch reaction: To each 2.0 ml microtube was added 1.8 ml of reaction feed. The reaction was incubated at 37° C. and rotated for 3.0 hr. 10 μl fractions were collected at 10, 30, 60, 120 and 180 minutes and analyzed by HPLC analysis. HPLC system: Thermo Scientific Vanquish Duo; Column: Acclaim Trinity P1; CAD detection. Solvent A: Acetonitrile; solvent B: 20 mM ammonium formate pH 4.5. 0-2 min (22% B); 2-4 min (22-35% B gradient); 4-6 min (35% B); 6-9 min (35-70% B gradient); 9-12 min (70% B); 12-14 min (70-22% B gradient); 14-15 min (22% B).

An 88% conversion was achieved after 180 min with the immobilized 1X enzyme system while a full conversion (100%) was achieved after 30 min with the immobilized 5X enzyme system (FIG. 7).

### Example 3c-D. Production of LSTa (in Batch)

LSTa was produced from LNT, Neu5Ac, ATP and CTP with system module 4 (SM4) using four elementary modules (EM1.4A, EM2.3D, EM3.1B, EM5.1A; see FIGS. 1&2) with NmCSS, PmPpa, CMPK, NDPK and PmST1 immobilized on S40W in a batch reactor.

BNC preparation: 2.0 g of S40W was scaffolded with 40 mg of magnetite nanoparticles (40 mg; 2% w/w) as described (“Enzyme Immobilization Method and Protocol”) onto which 1.6 mg NmCSS, 1.0 mg PmPpa, 4.0 mg CMPK, 4.0 mg NDPK and 2.5 mg PmST1 were added in 4.1 ml of 20 mM Tris pH 7.5. Bradford analysis showed that 98.2% of enzymes were immobilized. For the 1X immobilized enzyme reaction (Immob. Enzyme 1X) 1.8 ml of BNC suspension was transferred to 2.0 ml microtubes, the aqueous supernatant was removed, and the BNC was washed with two 1.0 ml volumes of water before adding the reaction feed. For the 5X immobilized enzyme reaction (Immob. Enzyme 5X) 9.0 ml of BNC suspension was transferred to a 15 ml Falcon tube, the aqueous supernatant was removed, and the BNC was washed with two 1.0 ml volumes of water while transferring the BNC suspension to a 2.0 ml microtube. 1.8 ml of reaction feed was added to both “Immob. Enzyme 1X” and “Immob. Enzyme 5X” containing tubes.

Preparation of reaction feed stock: The feedstock comprising 5 mM LNT, 10 mM Neu5Ac, 6 mM CTP and 15 mM ATP in 50 mM Tris pH 9.0 with 50 mM MgCl₂ was prepared by dissolving 35.4 mg of LNT, 34.6 mg of Neu5Ac, 33.3 mg of CTP and 88.1 mg of ATP in 8.0 ml of water followed by addition of 0.5 ml of Tris buffer (1M, pH 9.0) and 0.5 ml of 1 M MgCl₂. After the pH was adjusted to 9.0 with the addition of 1M NaOH, the final volume of feed stock was adjusted to 10 ml with additional water.

Batch reaction: To each 2.0 ml microtube was added 1.8 ml of reaction feed. The reaction was incubated at 37° C. and rotated for 1.0 hr. 10 μml fractions were collected at 10, 20, 30, 45 and 60 minutes and analyzed by HPLC analysis. HPLC system: Thermo Scientific Vanquish Duo; Column: Acclaim Trinity P1; CAD detection. Solvent A: Acetonitrile; solvent B: 50 mM ammonium formate pH 4.5. 0-4.5 min (35-50% B gradient); 4.5-6.5 min (50% B); 6.5-8 min (50-60% B gradient); 8-9 min (60-35% B gradient); 9-10 min (35% B).

A 94% conversion was achieved after 30 minutes with the immobilized 1X enzyme system, while a 96% conversion was achieved after 10 min with the immobilized 5X enzyme system (FIG. 8).

### Example 4: Cloning (Gene Constructs and Enzyme Production)

All enzymes in Table 2 were expressed recombinantly in Escherichia coli (BL21 DE3) either by the Bioexpression and Fermentation Facility (BFF) at the Complex Carbohydrate Research Center (CCRC) at the University of Georgia, Athens or by Zymtronix. Zymtronix produced all enzymes in-house using shake flasks grown in incubators and an FPLC unit (Akta Explorer, GE Healthcare) to purify HmFucT and Te2FT. All enzymes were purified from the soluble lysate by affinity chromatography (NiNTA), and the buffer was exchanged either by dialysis or size exclusion chromatography against 50 mM Tris pH 7.5. The enzymes were supplemented with 10% (w/w) glycerol and frozen at −80° C. for storage. Detailed experimental conditions related to expression and purification for each enzyme are provided.

TABLE 2
Summary of Enzyme Production

| Enzyme | | Organism | Uniprot | Yield^{b} | | Ref |
|---|---|---|---|---|---|---|
| PmST1 | α-2,3-sialyltransferase | Pasteurella multocida | Q9CNC4 | 431 | mg | 2 |
| NmCSS | α-2,3-sialic acid | Neisseria meningitidis | P0A0Z8 | 259 | mg | 3 |
| | synthetase | | | | | |
| HmFucT^{a} | α-1,2-fucosyltransferase | Helicobacter mustelae | D3UIY5 | 80 | mg | 4 |
| Te2FT^{a} | α-1,2-fucosyltransferase | Thermosynechococcus | Q8DK72 | 60 | mg | 5 |
| | | elongatus | | | | |
| BfFKP | Bifunctional L- | Bacteriodes fragilis | Q58T34 | 196 | mg | 6 |
| | fucokinase/GDP-L-Fuc | | | | | |
| | pyrophosphorylase | | | | | |
| PmPpa | Pyrophosphatase | Pasteurella multocida | P57918 | 480 | mg | 7 |
| NDPK | Nucleoside diphosphate | Escherichia coli | P0A763 | 357 | mg | 8 |
| | kinase | | | | | |
| CMPK | Cytidine | Escherichia coli | P0A6I0 | 435 | mg | 9 |
| | monophosphate kinase | | | | | |
| Bbh1 | β-N- | Bifidobacterium bifidum | D4QAP4 | 278 | mg | 10 |
| | acetylhexosaminidase | | | | | |

Table 2 Footnotes. ^{a} expressed as N-terminal eGFP fusion to enhance soluble expression and reduce aggregation. ^{b} Yield from 4 L fermentation volumes.

Table 2 References. For protein production and cloning; reference numbers indicated in Table 2.
1. Sugiarto, Go, et al. “Decreasing the sialidase activity of multifunctional Pasteurella multocida α2-3-sialyltransferase 1 (PmST1) by site-directed mutagenesis.” Molecular BioSystems 7.11 (2011): 3021-3027.
2. Yu, Hai, et al. “A multifunctional Pasteurella multocida sialyltransferase: a powerful tool for the synthesis of sialoside libraries.”Journal of the American Chemical Society 127.50 (2005): 17618-17619.
3. Karwaski, Marie-France, Warren W. Wakarchuk, and Michel Gilbert. “High-level expression of recombinant Neisseria CMP-sialic acid synthetase in Escherichia coli.” Protein expression and purification 25.2 (2002): 237-240.
4. Huang, Di, et al. “Metabolic engineering of Escherichia coli for the production of 2′-fucosyllactose and 3-fucosyllactose through modular pathway enhancement.” Metabolic engineering 41 (2017): 23-38.
5. Zhao, Chao, et al. “The one-pot multienzyme (OPME) synthesis of human blood group H antigens and a human milk oligosaccharide (HMOS) with highly active Thermosynechococcus elongatus al-2-fucosyltransferase.”Chemical Communications 52.20 (2016): 3899-3902.
6. Wang, Wei, et al. “Chemoenzymatic synthesis of GDP-L-fucose and the Lewis X glycan derivatives.” Proceedings of the National Academy of Sciences 106.38 (2009): 16096-16101.
7. Lau, Kam, et al. “Highly efficient chemoenzymatic synthesis of (31-4-linked galactosides with promiscuous bacterial β1-4-galactosyltransferases.” Chemical communications 46.33 (2010): 6066-6068.
8. Almaula, Niva, et al. “Nucleoside diphosphate kinase from Escherichia coli.” Journal of bacteriology 177.9 (1995): 2524-2529.
9. Bucurenci, Nadia, et al. “CMP kinase from Escherichia coli is structurally related to other nucleoside monophosphate kinases.” Journal of Biological Chemistry 271.5 (1996): 2856-2862.
10. Schmölzer, Katharina, et al. “Glycosynthase principle transformed into biocatalytic process technology: lacto-N-triose II production with engineered exo-hexosaminidase.” ACS Catalysis 9.6 (2019): 5503-5514.

All genes were synthesized by Genewiz (https://www.genewiz.com/en) and were cloned into a pET28a(+) vector (Novagen). The genetic sequence of the open reading frames and the corresponding translated sequence of all eight enzymes are listed below. Expression and purification details are listed.

### Example 4A: PmST1 Sequence and Method

Sequence

SEQ ID NO: 1
ATGCCGAGCAGCCATCATCATCATCATCACGATTACGATATCCCAACGA
CCGAAAACCTTTACTTCCAGGGATCCATGAAAACGATTACGCTGTACCT
CGATCCGGCCAGCCTCCCAGCGCTGAACCAACTGATGGACTTCACGCAG
AACAACGAGGACAAAACGCACCCGCGCATCTTCGGTCTGAGTCGCTTTA
AAATCCCGGATAACATTATCACCCAGTACCAGAACATCCACTTTGTGGA
ACTGAAGGACAATCGCCCGACCGAAGCGCTGTTCACCATCCTCGATCAG
TACCCGGGCAACATTGAGCTGAATATCCATCTGAATATTGCGCATAGCG
TGCAGCTGATCCGCCCGATTCTGGCCTACCGCTTCAAACATCTGGATCG
CGTTAGCATCCAGCAGCTGAACCTCTACGATGATGGCAGCATGGAGTAC
GTTGATCTGGAGAAGGAGGAGAATAAGGATATCAGTGCGGAGATCAAAC
AAGCCGAGAAGCAGCTGAGCCACTATCTGCTGACCGGCAAGATCAAGTT
CGATAACCCGACCATCGCCCGCTACGTTTGGCAGAGCGCCTTTCCGGTG
AAATACCACTTTCTGAGCACCGACTACTTTGAGAAGGCGGAATTTCTGC
AGCCGCTGAAGGAATATCTGGCGGAGAACTACCAGAAGATGGATTGGAC
CGCGTACCAGCAACTGACCCCAGAGCAACAAGCCTTTTATCTCACGCTG
GTGGGTTTCAACGACGAGGTGAAACAGAGTCTGGAAGTGCAGCAAGCCA
AGTTCATCTTCACGGGTACCACCACGTGGGAAGGTAACACCGACGTTCG
CGAATACTACGCGCAGCAGCAGCTCAATCTGCTGAATCACTTCACGCAA
GCCGAAGGCGATCTGTTCATCGGTGACCACTACAAAATTTACTTCAAAG
GTCACCCGCGCGGCGGCGAGATCAACGATTATATTCTGAACAACGCGAA
AAATATCACGAACATCCCGGCCAACATCAGCTTTGAAGTGCTGATGATG
ACGGGTCTGCTGCCAGATAAGGTTGGCGGTGTTGCCAGCAGTCTGTACT
TCAGCCTCCCAAAAGAAAAAATCAGCCATATCATCTTTACGAGTAATAA
ACAAGTGAAAAGCAAGGAGGATGCGCTGAACAATCCGTACGTGAAGGTG
ATGCGCCGCCTCGGCATCATCGATGAGAGCCAAGTTATCTTCTGGGACA
GTCTGAAGCAGCTGTAA
MPSSHHHHHHDYDIPTTENLYFQGSMKTITLYLDPASLPALNQLMDFTQ
NNEDKTHPRIFGLSRFKIPDNIITQYQNIHFVELKDNRPTEALFTILDQ
YPGNIELNIHLNIAHSVQLIRPILAYRFKHLDRVSIQQLNLYDDGSMEY
VDLEKEENKDISAEIKQAEKQLSHYLLTGKIKFDNPTIARYVWQSAFPV
KYHELSTDYFEKAEFLQPLKEYLAENYQKMDWTAYQQLTPEQQAFYLTL
VGFNDEVKQSLEVQQAKFIFTGTTTWEGNTDVREYYAQQQLNLLNHFTQ
AEGDLFIGDHYKIYFKGHPRGGEINDYILNNAKNITNIPANISFEVLMM
TGLLPDKVGGVASSLYFSLPKEKISHIIFTSNKQVKSKEDALNNPYVKV
MRRLGIIDESQVIFWDSLKQL*

Method

Expression: TB media, 0.2 mM IPTG, 20 hrs, 25° C.

Lysis buffer: 20 mM Tris, pH 7.5, 500 mM NaCl, 20 mM imidazole

Affinity Chromatography:
a) Loading of spun down lysate.
b) Gradient: 20 mM Tris, pH 7.5, 500 mM NaCl, 20-500 mM imidazole, 10CV

Size exclusion chromatography: Final buffer formulation: 25 mM Tris pH 8.0, 10% glycerol

Final yield: 431 mg from 4L expression(fermentation), 4.79 mg/ml

### Example 4B: NmCSS Sequence and Method

Sequence

SEQ ID NO: 2
ATGCCGAGCAGCCATCATCATCATCATCACGATTACGATATCCCAACGA
CCGAAAACCTTTACTTCCAGGGATCCATGGAGAAACAGAATATCGCGGT
GATTCTGGCCCGCCAGAATAGCAAAGGTCTGCCGCTGAAGAATCTGCGC
AAAATGAACGGCATCAGTCTGCTGGGCCATACCATCAATGCCGCGATCA
GCAGCAAGTGCTTCGACCGCATTATCGTTAGCACCGATGGCGGTCTGAT
TGCCGAAGAAGCGAAAAACTTCGGCGTGGAGGTTGTTCTGCGCCCAGCG
GAACTCGCGAGTGATACCGCCAGCAGCATTAGCGGCGTTATTCACGCGC
TGGAGACGATTGGCAGCAATAGCGGCACGGTGACGCTGCTGCAACCAAC
CAGCCCGCTGCGTACCGGTGCCCATATTCGCGAGGCCTTCAGCCTCTTC
GACGAGAAGATCAAAGGCAGCGTGGTTAGCGCGTGCCCGATGGAACATC
ACCCGCTGAAAACGCTGCTGCAGATCAACAATGGCGAGTACGCCCCAAT
GCGCCATCTGAGCGATCTGGAGCAGCCGCGTCAGCAACTGCCACAAGCC
TTCCGCCCGAACGGCGCCATCTACATCAATGACACCGCGAGTCTGATCG
CCAACAACTGCTTCTTCATCGCGCCGACGAAGCTGTACATCATGAGCCA
TCAAGATAGCATCGACATCGATACCGAGCTGGATCTGCAGCAAGCCGAG
AACATTCTGAACCACAAAGAGAGCTAA
MPSSHHHHHHDYDIPTTENLYFQGSMEKQNIAVILARQNSKGLPLKNLR
KMNGISLLGHTINAAISSKCFDRIIVSTDGGLIAEEAKNFGVEVVLRPA
ELASDTASSISGVIHALETIGSNSGTVTLLQPTSPLRTGAHIREAFSLF
DEKIKGSVVSACPMEHHPLKTLLQINNGEYAPMRHLSDLEQPRQQLPQA
FRPNGAIYINDTASLIANNCFFIAPTKLYIMSHQDSIDIDTELDLQQAE
NILNHKES*

Method

Expression: LB media, 0.1 mM IPTG, 6 hrs, 37° C.

Lysis Buffer: 50 mM Tris, pH 7.6.

Affinity Chromatography:
a) Loading of spun down lysate.
b) Gradient: 50 mM Tris, pH 7.6, 300 mM NaCl, 20-500 mM imidazole, 10CV

Size exclusion chromatography: Final buffer formulation: 20 mM Tris pH 7.5, 10% glycerol.

Final yield: 259 mg from 4 L expression (fermentation), 1.33 mg/ml.

### Example 4C: HmFucT Sequence and Method

Sequence

SEQ ID NO: 3
ATGGCAAGCAGCCATCATCATCATCATCATGGCAGCAGCGTGAGCAAAG
GCGAAGAACTGTTTACCGGCGTGGTGCCGATTCTGGTGGAACTGGATGG
CGATGTGAACGGCCATAAATTTAGCGTGCGCGGCGAAGGCGAAGGCGAT
GCGACCAACGGCAAACTGACCCTGAAATTTATTTGCACCACCGGCAAAC
TGCCGGTGCCGTGGCCGACCCTGGTGACCACCCTGACCTATGGCGTGCA
GTGCTTTAGCCGCTATCCGGATCATATGAAACAGCATGATTTTTTTAAA
AGCGCGATGCCGGAAGGCTATGTGCAAGAACGCACCATTAGCTTTAAAG
ATGATGGCACCTATAAAACCCGCGCGGAAGTGAAATTTGAAGGCGATAC
CCTGGTGAACCGCATTGAACTGAAAGGCATTGATTTTAAAGAAGATGGC
AACATTCTGGGCCATAAACTGGAATATAACTTTAACAGCCATAACGTGT
ATATTACCGCGGATAAACAGAAAAACGGCATTAAAGCGAACTTTAAAAT
TCGCCATAACGTGGAAGATGGCAGCGTGCAGCTGGCGGATCATTATCAG
CAGAACACCCCGATTGGCGATGGCCCGGTGCTGCTGCCGGATAACCATT
ATCTGAGCACGCAGAGCAAACTGAGCAAAGATCCGAACGAAAAACGCGA
TCATATGGTGCTGCTGGAATTTGTGACCGCGGCGGGCATTACCCTGGGC
ATGGATGAACTGTATAAACTGGGATCCGCGAGCATGGATTTTAAAATTG
TGCAAGTGCATGGCGGCCTGGGCAATCAGATGTTTCAGTATGCGTTTGC
GAAAAGCCTGCAGACCCATCTGAACATTCCGGTGCTGCTGGATACCACC
TGGTTTGATTATGGCAACCGCGAACTGGGCCTGCATCTGTTTCCGATTG
ATCTGCAGTGCGCGAGCGCGCAGCAGATTGCTGCGGCGCATATGCAGAA
CCTGCCGCGCCTGGTCCGCGGAGCGCTTCGCCGCATGGGCCTGGGCCGC
GTGAGCAAAGAAATTGTGTTTGAATATATGCCGGAACTGTTTGAACCGA
GCCGCATTGCGTATTTTCATGGCTATTTTCAAGATCCGCGCTATTTTGA
AGATATTAGCCCGCTGATTAAACAGACCTTTACCCTGCCGCATCCGACC
GAACATGCGGAACAGTATAGCCGCAAACTGAGTCAGATTCTGGCGGCGA
AAAACAGCGTGTTTGTGCATATTCGCCGCGGCGATTATATGCGCCTGGG
CTGGCAGCTGGATATTAGCTATCAGCTGCGCGCGATTGCGTATATGGCG
AAACGCGTACAGAACCTGGAACTGTTTCTGTTTTGCGAAGATCTGGAAT
TTGTTCAGAACCTGGATCTGGGCTATCCGTTTGTGGATATGACCACCCG
CGATGGCGCGGCGCATTGGGATATGATGCTGATGCAGAGCTGCAAACAT
GGCATTATTACCAACAGCACCTATAGCTGGTGGGCGGCGTATCTGATTA
AAAACCCGGAAAAAATTATTATTGGCCCGAGCCATTGGATTTATGGCAA
CGAAAACATTCTGTGCAAAGATTGGGTGAAAATTGAAAGTCAGTTTGAA
ACCAAAAGCTAA
MASSHHHHHHGSSVSKGEELFTGVVPILVELDGDVNGHKFSVRGEGEGD
ATNGKLTLKFICTTGKLPVPWPTLVTTLTYGVQCFSRYPDHMKQHDFFK
SAMPEGYVQERTISFKDDGTYKTRAEVKFEGDTLVNRIELKGIDFKEDG
NILGHKLEYNFNSHNVYITADKQKNGIKANFKIRHNVEDGSVQLADHYQ
QNTPIGDGPVLLPDNHYLSTQSKLSKDPNEKRDHMVLLEFVTAAGITLG
MDELYKLGSASMDFKIVQVHGGLGNQMFQYAFAKSLQTHLNIPVLLDTT
WFDYGNRELGLHLFPIDLQCASAQQIAAAHMQNLPRLVRGALRRMGLGR
VSKEIVFEYMPELFEPSRIAYFHGYFQDPRYFEDISPLIKQTFTLPHPT
EHAEQYSRKLSQILAAKNSVFVHIRRGDYMRLGWQLDISYQLRAIAYMA
KRVQNLELFLFCEDLEFVQNLDLGYPFVDMTTRDGAAHWDMMLMQSCKH
GIITNSTYSWWAAYLIKNPEKIIIGPSHWIYGNENILCKDWVKIESQFE
TKS*

Method

Expression: TB+0.5 M Sorbitol media, 0.1 mM IPTG, 2 days, 8° C.

Lysis: Buffer: 50 mM Sodium phosphate pH 7.4, 300 mM NaCl, 20 mM Imidazole, 5 mM MgCl2, 5 mM BME, 200 mM L-Arginine, 0.25 mg/mL Lysozyme, 0.02 mg/mL DNase I, 1 mM PMSF. 60 min incubation, Sonicate ½″ probe, 90% amplitude, 5 s on, 10 s off, 2 min process time.

Affinity Chromatography: Dilution Buffer: 50 mM Sodium phosphate pH 7.4, 300 mM NaCl, 20 mM Imidazole, 5 mM MgCl₂, 5 mM BME, 10% glycerol. Dilute lysate 1:1 with dilution buffer. Buffer A: 50 mM Sodium phosphate pH 7.4, 300 mM NaCl, 20 mM Imidazole, 5 mM MgCl2, 5 mM BME, 100 mM L-Arginine, 10% glycerol. Buffer B: 50 mM Sodium phosphate pH 7.4, 300 mM NaCl, 3000 mM Imidazole, 5 mM MgCl₂, 5 mM BME, 200 mM L-Arginine, 10% glycerol. 10 mL Ni-NTA Gravity Column. 5 CV equil, Load, 60 min incubation, 5 CV wash (0% B), 5 CV 100% B.

Size Exclusion Chromatography: Final buffer formulation: 25 mM Tris pH 8.0, 300 mM NaCl, 10 mM MgCl2, 10% glycerol. 0.3 mL/min, 1 mL load. Superdex 200 Increase 10/300 GL.

Final yield: 20 mg per 1L expression, 0.89 mg/ml.

### Example 4D: Te2FT Sequence and Method

Sequence

SEQ ID NO: 4
ATGAAATCTTCCCACCATCACCATCACCATGGTTCTTCTGTGAGCAAGG
GCGAGGAGCTGTTCACCGGGGTGGTGCCCATCCTGGTCGAGCTGGACGG
CGACGTAAACGGCCACAAGTTCAGCGTGCGCGGCGAGGGCGAGGGCGAT
GCCACCAACGGCAAGCTGACCCTGAAGTTCATCTGCACCACCGGCAAGC
TGCCCGTGCCCTGGCCCACCCTCGTGACCACCCTGACCTACGGCGTGCA
GTGCTTCAGCCGCTACCCCGACCACATGAAGCAGCACGACTTCTTCAAG
TCCGCCATGCCCGAAGGCTACGTCCAGGAGCGCACCATCTCCTTCAAGG
ACGACGGCACCTACAAGACCCGCGCCGAGGTGAAGTTCGAGGGCGACAC
CCTGGTGAACCGCATCGAGCTGAAGGGCATCGACTTCAAGGAGGACGGC
AACATCCTGGGGCACAAGCTGGAGTACAACTTCAACAGCCACAACGTCT
ATATCACGGCCGACAAGCAGAAGAACGGCATCAAGGCGAACTTCAAGAT
CCGCCACAACGTCGAGGACGGCAGCGTGCAGCTCGCCGACCACTACCAG
CAGAACACCCCCATCGGCGACGGCCCCGTGCTGCTGCCCGACAACCACT
ACCTGAGCACCCAGTCCAAGCTGAGCAAAGACCCCAACGAGAAGCGCGA
TCACATGGTCCTGCTGGAGTTCGTGACCGCCGCCGGGATCACTCTCGGC
ATGGACGAGCTGTACAAGCTCGGGTCCGACCTAGTACCACGTGGATCGA
TGATTATCGTACATTTGTGCGGAGGATTGGGGAACCAAATGTTTCAATA
TGCAGCTGGACTGGCAGCGGCGCATCGTATCGGATCTGAAGTAAAATTT
GACACTCATTGGTTTGATGCCACGTGTCTGCACCAAGGCCTTGAGTTGC
GTCGCGTATTCGGTTTAGAGCTGCCCGAACCTTCCTCAAAGGATTTGCG
TAAGGTACTGGGGGCGTGCGTTCATCCGGCCGTTCGCCGCTTACTGGCT
GGCCACTTTCTTCATGGCTTACGTCCGAAGAGCCTTGTCATTCAGCCGC
ATTTTCATTATTGGACTGGGTTTGAGCACCTGCCGGACAATGTATATTT
AGAGGGCTACTGGCAATCGGAGCGCTATTTCTCGAACATTGCTGATATT
ATTCGCCAACAGTTTCGCTTTGTAGAACCGTTGGACCCTCATAACGCTG
CTTTGATGGACGAAATGCAGTCCGGTGTCAGTGTGAGTCTGCATATCCG
CCGTGGTGACTACTTCAACAATCCCCAGATGCGCCGTGTACATGGCGTT
GATCTGTCTGAATATTATCCGGCTGCTGTCGCCACGATGATTGAGAAGA
CAAATGCCGAGCGTTTTTACGTGTTCAGCGATGATCCCCAGTGGGTCTT
AGAGCATCTGAAGTTGCCGGTATCGTACACCGTTGTAGATCATAATCGT
GGAGCTGCCTCTTATCGTGATATGCAATTGATGTCCGCTTGTCGCCATC
ACATTATCGCTAATAGCACCTTCTCGTGGTGGGGGGCATGGCTGAATCC
TCGCCCTGACAAAGTAGTGATTGCGCCCCGCCATTGGTTTAACGTGGAC
GTGTTTGATACTCGTGATTTGTACTGTCCTGGATGGATTGTTCTGTAA
MKSSHHHHHHGSSVSKGEELFTGVVPILVELDGDVNGHKFSVRGEGEGD
ATNGKLTLKFICTTGKLPVPWPTLVTTLTYGVQCFSRYPDHMKQHDFFK
SAMPEGYVQERTISFKDDGTYKTRAEVKFEGDTLVNRIELKGIDFKEDG
NILGHKLEYNFNSHNVYITADKQKNGIKANFKIRHNVEDGSVQLADHYQ
QNTPIGDGPVLLPDNHYLSTQSKLSKDPNEKRDHMVLLEFVTAAGITLG
MDELYKLGSDLVPRGSMIIVHLCGGLGNQMFQYAAGLAAAHRIGSEVKF
DTHWFDATCLHQGLELRRVFGLELPEPSSKDLRKVLGACVHPAVRRLLA
GHFLHGLRPKSLVIQPHFHYWTGFEHLPDNVYLEGYWQSERYFSNIADI
IRQQFRFVEPLDPHNAALMDEMQSGVSVSLHIRRGDYFNNPQMRRVHGV
DLSEYYPAAVATMIEKTNAERFYVFSDDPQWVLEHLKLPVSYTVVDHNR
GAASYRDMQLMSACRHHIIANSTFSWWGAWLNPRPDKVVIAPRHWFNVD
VFDTRDLYCPGWIVL*

Method

Expression: TB media, 0.2 mM IPTG, 20 hrs, 18° C.

Lysis: Buffer: 50 mM Sodium phosphate pH 7.4, 300 mM NaCl, 20 mM Imidazole, 5 mM BME, 1% Triton X-100, 25 mg Lysozyme, 1 mM PMSF. 45 min incubation, Sonicate ¼″ probe, 40% amplitude, 10 s on, 30 s off, 2 min process time.

Affinity Chromatography: Buffer A: 50 mM Sodium phosphate pH 7.4, 300 mM NaCl, 20 mM Imidazole, 5 mM BME. Buffer B: 50 mM Sodium phosphate pH 7.4, 300 mM NaCl, 500 mM Imidazole, 5 mM BME. 10 mL Ni-NTA Gravity Column. 10 CV equil, Load, 45 min incubation, 10 CV wash (0% B), 2 CV wash (2.5% B), 5 CV 100% B.

Dialysis: Dialysis Buffer: 20 mM Tris pH 8.6. 12-14 kDa cutoff dialysis tubing. 4° C. overnight.

Anion Exchange: Buffer A: 20 mM Tris pH 8.6. Buffer B: 20 mM Tris pH 8.6, 1 M NaCl. 5 CV equil, Load, 10 CV wash, 10 CV 0-100% B.

Desalting: Final buffer formulation: 25 mM Tris pH 8.0, 10% glycerol. HiPrep 26/10 Desalting.

Final yield: 15.174 mg from 1L expression, 1.686 mg/ml

### Example 4E: BfFKP Sequence and Method

Sequence

SEQ ID NO: 5
ATGCCGAGCAGCCATCATCATCATCATCACGATTACGATATCCCAACGA
CCGAAAACCTTTACTTCCAGGGATCCATGCAAAAACTGCTGAGCCTGCC
GAGCAATCTGGTTCAAAGCTTCCACGAACTGGAGCGTGTTAATCGCACC
GACTGGTTCTGCACCAGCGATCCGGTTGGCAAGAAACTGGGTAGCGGTG
GCGGTACCAGCTGGCTGCTGGAGGAATGCTACAACGAGTATAGCGACGG
CGCGACCTTCGGTGAATGGCTGGAGAAGGAAAAACGTATCCTGCTGCAT
GCGGGCGGTCAGAGCCGTCGTCTGCCGGGCTACGCGCCGAGCGGTAAAA
TTCTGACCCCGGTGCCGGTTTTTCGTTGGGAGCGTGGCCAGCACCTGGG
TCAAAACCTGCTGAGCCTGCAGCTGCCGCTGTATGAAAAGATCATGAGC
CTGGCGCCGGATAAACTGCACACCCTGATCGCGAGCGGTGACGTTTACA
TTCGTAGCGAGAAGCCGCTGCAAAGCATTCCGGAAGCGGATGTGGTTTG
CTATGGTCTGTGGGTTGACCCGAGCCTGGCGACCCACCATGGTGTGTTC
GCGAGCGATCGTAAGCACCCGGAGCAGCTGGACTTTATGCTGCAAAAAC
CGAGCCTGGCGGAGCTGGAAAGCCTGAGCAAGACCCACCTGTTCCTGAT
GGATATCGGTATTTGGCTGCTGAGCGACCGTGCGGTGGAGATCCTGATG
AAGCGTAGCCACAAAGAAAGCAGCGAGGAACTGAAATACTATGACCTGT
ACAGCGATTTTGGTCTGGCGCTGGGTACCCACCCGCGTATCGAAGATGA
GGAAGTGAACACCCTGAGCGTTGCGATTCTGCCGCTGCCGGGCGGTGAG
TTTTACCACTATGGTACCAGCAAAGAACTGATCAGCAGCACCCTGAGCG
TGCAGAACAAGGTTTACGACCAACGTCGTATTATGCACCGTAAGGTTAA
ACCGAACCCGGCGATGTTCGTGCAGAACGCGGTGGTTCGTATCCCGCTG
TGCGCGGAGAACGCGGATCTGTGGATCGAAAACAGCCACATTGGCCCGA
AGTGGAAAATTGCGAGCCGTCACATCATTACCGGTGTTCCGGAGAACGA
CTGGAGCCTGGCGGTGCCGGCGGGCGTGTGCGTTGATGTGGTTCCGATG
GGCGACAAAGGTTTCGTTGCGCGTCCGTACGGCCTGGACGATGTGTTTA
AGGGTGACCTGCGTGATAGCAAAACCACCCTGACCGGCATCCCGTTCGG
TGAATGGATGAGCAAGCGTGGCCTGAGCTATACCGATCTGAAAGGTCGT
ACCGACGATCTGCAAGCTGTGAGCGTTTTTCCGATGGTGAACAGCGTTG
AGGAACTGGGTCTGGTTCTGCGTTGGATGCTGAGCGAGCCGGAACTGGA
GGAAGGCAAAAACATCTGGCTGCGTAGCGAGCACTTCAGCGCGGACGAA
ATTAGCGCGGGTGCGAACCTGAAGCGTCTGTACGCGCAGCGTGAGGAAT
TTCGTAAGGGCAACTGGAAAGCGCTGGCGGTGAACCACGAGAAGAGCGT
TTTCTATCAACTGGACCTGGCGGATGCGGCGGAAGATTTTGTGCGTCTG
GGTCTGGACATGCCGGAGCTGCTGCCGGAAGATGCGCTGCAGATGAGCC
GTATCCACAACCGTATGCTGCGTGCGCGTATTCTGAAGCTGGACGGTAA
AGATTACCGTCCGGAGGAACAAGCGGCGTTCGACCTGCTGCGTGATGGC
CTGCTGGACGGTATCAGCAACCGTAAGAGCACCCCGAAACTGGATGTGT
ATAGCGACCAGATCGTTTGGGGTCGTAGCCCGGTGCGTATTGATATGGC
GGGCGGTTGGACCGACACCCCGCCGTACAGCCTGTATAGCGGCGGTAAC
GTGGTTAACCTGGCGATCGAGCTGAACGGTCAGCCGCCGCTGCAAGTGT
ACGTTAAGCCGTGCAAAGATTTTCACATCGTTCTGCGTAGCATTGACAT
GGGCGCGATGGAGATTGTGAGCACCTTCGATGAACTGCAGGACTACAAG
AAAATCGGTAGCCCGTTTAGCATTCCGAAAGCGGCGCTGAGCCTGGCGG
GTTTTGCGCCGGCGTTTAGCGCGGTTAGCTATGCGAGCCTGGAGGAACA
ACTGAAGGATTTCGGTGCGGGTATTGAGGTGACCCTGCTGGCGGCGATT
CCGGCGGGTAGCGGTCTGGGTACCAGCAGCATCCTGGCGAGCACCGTTC
TGGGTGCGATTAACGATTTTTGCGGTCTGGCGTGGGACAAAAACGAGAT
CTGCCAGCGTACCCTGGTGCTGGAACAACTGCTGACCACCGGCGGTGGC
TGGCAGGACCAATACGGTGGCGTGCTGCAGGGCGTTAAGCTGCTGCAAA
CCGAAGCGGGTTTTGCGCAGAGCCCGCTGGTTCGTTGGCTGCCGGATCA
CCTGTTTACCCACCCGGAATATAAAGACTGCCACCTGCTGTACTATACC
GGCATCACCCGTACCGCGAAGGGTATCCTGGCGGAGATTGTGAGCAGCA
TGTTCCTGAACAGCAGCCTGCACCTGAACCTGCTGAGCGAAATGAAGGC
GCACGCGCTGGATATGAACGAGGCGATCCAGCGTGGTAGCTTCGTTGAA
TTTGGCCGTCTGGTGGGTAAAACCTGGGAGCAAAACAAAGCGCTGGACA
GCGGTACCAACCCGCCGGCGGTGGAAGCGATCATTGATCTGATTAAGGA
CTACACCCTGGGCTATAAACTGCCGGGTGCGGGTGGCGGTGGCTACCTG
TATATGGTTGCGAAGGACCCGCAGGCGGCGGTGCGTATCCGTAAAATTC
TGACCGAGAACGCGCCGAACCCGCGTGCGCGTTTCGTTGAGATGACCCT
GAGCGATAAAGGTTTCCAAGTGAGCCGCAGCTAA
MPSSHHHHHHDYDIPTTENLYFQGSMQKLLSLPSNLVQSFHELERVNRT
DWFCTSDPVGKKLGSGGGTSWLLEECYNEYSDGATFGEWLEKEKRILLH
AGGQSRRLPGYAPSGKILTPVPVFRWERGQHLGQNLLSLQLPLYEKIMS
LAPDKLHTLIASGDVYIRSEKPLQSIPEADVVCYGLWVDPSLATHHGVF
ASDRKHPEQLDFMLQKPSLAELESLSKTHLFLMDIGIWLLSDRAVEILM
KRSHKESSEELKYYDLYSDFGLALGTHPRIEDEEVNTLSVAILPLPGGE
FYHYGTSKELISSTLSVQNKVYDQRRIMHRKVKPNPAMFVQNAVVRIPL
CAENADLWIENSHIGPKWKIASRHIITGVPENDWSLAVPAGVCVDVVPM
GDKGFVARPYGLDDVFKGDLRDSKTTLTGIPFGEWMSKRGLSYTDLKGR
TDDLQAVSVFPMVNSVEELGLVLRWMLSEPELEEGKNIWLRSEHFSADE
ISAGANLKRLYAQREEFRKGNWKALAVNHEKSVFYQLDLADAAEDFVRL
GLDMPELLPEDALQMSRIHNRMLRARILKLDGKDYRPEEQAAFDLLRDG
LLDGISNRKSTPKLDVYSDQIVWGRSPVRIDMAGGWTDTPPYSLYSGGN
VVNLAIELNGQPPLQVYVKPCKDFHIVLRSIDMGAMEIVSTFDELQDYK
KIGSPFSIPKAALSLAGFAPAFSAVSYASLEEQLKDFGAGIEVTLLAAI
PAGSGLGTSSILASTVLGAINDFCGLAWDKNEICQRTLVLEQLLTTGGG
WQDQYGGVLQGVKLLQTEAGFAQSPLVRWLPDHLFTHPEYKDCHLLYYT
GITRTAKGILAEIVSSMFLNSSLHLNLLSEMKAHALDMNEAIQRGSFVE
FGRLVGKTWEQNKALDSGTNPPAVEAIIDLIKDYTLGYKLPGAGGGGYL
YMVAKDPQAAVRIRKILTENAPNPRARFVEMTLSDKGFQVSRS*

Method

Expression: LB media, 0.1 mM IPTG, 20 hrs, 16° C.

Lysis Buffer: 20 mM Tris, pH 8.0, 500 mM NaCl, 20 mM imidazole, 0.1% Triton-X100 Affinity Chromatography:
a) Loading of spun down lysate.
b) Gradient: 20 mM Tris, pH 8.0, 500 mM NaCl, 20-500 mM imidazole, 10% glycerol, 10CV.

Size exclusion chromatography: Final buffer formulation: 20 mM Tris pH 8.0, 50 mM NaCl, 10% glycerol.

Final yield: 196 mg from 4L expression (fermentation), 1.45 mg/ml

### Example 4F: PmPpa Sequence and Method

Sequence

SEQ ID NO: 6
ATGGCCAGCAGCATGGGTCTGGAAACCGTTCCAGCCGGTAAAGCGCTGC
CGGACGACATCTACGTGGTTATTGAGATCCCGGCGAACAGCGACCCGAT
CAAGTATGAAGTGGACAAGGAGAGCGGCGCGCTGTTCGTGGATCGCTTC
ATGGCCACCGCGATGTTTTATCCGGCCAACTACGGCTACGTGAACAATA
CCCTCAGTCTGGATGGCGATCCAGTGGATGTTCTCGTGCCAACCCCATA
CCCACTGCAGCCGGGCAGCGTTATTCGCTGCCGTCCGGTTGGCGTGCTG
AAAATGACCGATGAAGCGGGCAGCGATGCGAAAGTTGTGGCGGTTCCGC
ATAGCAAGCTGACCAAGGAGTACGACCACATCAAAGACGTGAATGATCT
GCCGGCGCTGCTGAAAGCCCAGATCCAGCACTTCTTCGAGAGCTACAAA
GCGCTGGAAGCCGGTAAATGGGTGAAAGTGGATGGCTGGGAAGGCGTTG
ATGCGGCCCGCCAAGAGATTCTGGATAGCTTCGAACGCGCGAAGAAACT
CGAGCACCACCACCACCACCACTGA
MASSMGLETVPAGKALPDDIYVVIEIPANSDPIKYEVDKESGALFVDRF
MATAMFYPANYGYVNNTLSLDGDPVDVLVPTPYPLQPGSVIRCRPVGVL
KMTDEAGSDAKVVAVPHSKLTKEYDHIKDVNDLPALLKAQIQHFFESYK
ALEAGKWVKVDGWEGVDAARQEILDSFERAKKLEHHHHHH*

Method

Expression: LB media, 0.1 mM IPTG, 20 hrs, 20° C.

Lysis buffer: 50 mM Tris, pH 7.5, 500 mM NaCl, 5 mM imidazole Affinity Chromatography:
a) Loading of spun down lysate.
b) Gradient: 50 mM Tris, pH 7.5, 500 mM NaCl, 20-250 mM imidazole, 10CV.

Size exclusion chromatography: Final buffer formulation: 20 mM Tris pH 7.5, 10% glycerol.

Final yield: 480 mg from 4L expression (fermentation), 5.78 mg/ml

### Example 4G: CMPK Sequence and Method

Sequence

SEQ ID NO: 7
ATGGGCAGCAGCCATCATCATCATCACCACAGCAGCGGTCTGGTTCCAC
GTGGCAGCATGACCGCCATCGCCCCAGTTATCACCATTGATGGCCCGAG
CGGTGCGGGTAAAGGTACGCTGTGCAAAGCGATGGCGGAAGCGCTGCAA
TGGCATCTGCTGGATAGCGGCGCCATCTATCGCGTTCTCGCGCTGGCGG
CGCTGCATCATCACGTGGATGTGGCGAGCGAAGACGCGCTGGTTCCGCT
GGCCAGCCATCTGGATGTGCGCTTTGTGAGCACCAACGGCAATCTGGAG
GTTATTCTGGAGGGCGAAGATGTTAGCGGCGAAATCCGCACGCAAGAAG
TTGCCAATGCGGCGAGTCAAGTTGCCGCCTTTCCACGCGTTCGTGAAGC
GCTGCTGCGCCGTCAGCGTGCCTTTCGTGAACTGCCGGGTCTGATCGCC
GATGGTCGTGATATGGGCACGGTTGTGTTCCCGGATGCCCCGGTGAAAA
TCTTTCTGGACGCCAGCAGTGAAGAACGTGCGCACCGCCGTATGCTGCA
GCTGCAAGTTAAGGGTTTCAGCGTGAACTTCGAGCGTCTGCTGGCGGAA
ATCAAAGAACGCGATGACCGCGACCGCAATCGTGCCGTTGCCCCACTGG
TTCCAGCGGCCGATGCCCTCGTTCTGGATAGCACGACGCTGAGCATCGA
ACAAGTTATTGAGAAAGCGCTGCAGTACGCCCGCCAGAAACTGGCGCTG
GCCTAA
MGSSHHHHHHSSGLVPRGSMTAIAPVITIDGPSGAGKGTLCKAMAEALQ
WHLLDSGAIYRVLALAALHHHVDVASEDALVPLASHLDVRFVSTNGNLE
VILEGEDVSGEIRTQEVANAASQVAAFPRVREALLRRQRAFRELPGLIA
DGRDMGTVVFPDAPVKIFLDASSEERAHRRMLQLQVKGFSVNFERLLAE
IKERDDRDRNRAVAPLVPAADALVLDSTTLSIEQVIEKALQYARQKLAL
A*

Method

Expression: LB media, 0.5 mM IPTG, 20 hrs, 18° C.

Lysis buffer: 20 mM Tris, pH 8.0, 500 mM NaCl, 20 mM imidazole Affinity Chromatography:
a) Loading of spun down lysate.
b) Elution: 20 mM Tris, pH 8.0, 500 mM NaCl, 20-500 mM imidazole, 10CV Size exclusion chromatography: Final buffer formulation: 20 mM Tris pH 8.0, 10% glycerol.

Final yield: 435 mg from 4L expression (fermentation), 2.56 mg/ml

### Example 4H: NDPK Sequence and Method

Sequence

SEQ ID NO: 8
ATGGGCAGCAGCCATCATCATCATCATCACAGCAGCGGCCTGGTGCCGC
GCGGCAGCATGGCCATCGAGCGCACCTTTAGCATCATCAAGCCAAACGC
CGTTGCGAAGAACGTGATCGGCAACATCTTCGCCCGTTTCGAAGCCGCC
GGTTTCAAAATCGTGGGCACCAAGATGCTGCATCTGACCGTTGAACAAG
CCCGCGGCTTTTACGCGGAACACGATGGCAAACCGTTCTTCGACGGTCT
GGTGGAATTCATGACCAGCGGCCCGATCGTGGTTAGCGTGCTGGAAGGC
GAAAATGCCGTTCAGCGCCATCGCGATCTGCTGGGTGCGACCAATCCAG
CCAATGCGCTGGCGGGTACGCTGCGTGCGGATTACGCCGACAGCCTCAC
CGAAAACGGCACGCATGGCAGCGATAGCGTGGAAAGCGCCGCCCGTGAA
ATCGCCTATTTCTTCGGCGAGGGTGAAGTGTGCCCACGTACGCGCTAA
MGSSHHHHHHSSGLVPRGSMAIERTFSIIKPNAVAKNVIGNIFARFEAA
GFKIVGTKMLHLTVEQARGFYAEHDGKPFFDGLVEFMTSGPIVVSVLEG
ENAVQRHRDLLGATNPANALAGTLRADYADSLTENGTHGSDSVESAARE
IAYFFGEGEVCPRTR*

Method

Expression: LB media, 0.4 mM IPTG, 3 hrs, 37° C.

Lysis buffer: 50 mM Tris, pH 7.9, 500 mM NaCl, 20 mM imidazole, 10 mM (3Me Affinity Chromatography:
a) Loading of spun down lysate.
b) Gradient: 50 mM Tris, pH 7.9, 500 mM NaCl, 20-250 mM imidazole, 10 mM βMe, 10CV

Size exclusion chromatography: Final buffer formulation: 20 mM Tris pH 8.0, 10% glycerol

Final yield: 357 mg from 4L expression (fermentation), 6.13 mg/ml

### Example 41: Bbh1 Sequence and Method

Sequence

SEQ ID NO: 9
ATGGGAAGTGATGATAATCTGGCGCTGAACCAAACGGTTACCGCGAGCA
GTTACGAGGTTGCCACCACCGCGCCGGAAAAGGCCGTGGATGGTGATCT
GGGCACCCGTTGGGGTACCGCCCAGAATAAAGCCGCGAATGAGTGGATT
GAAGTTGGTCTGGGCGGTACGAAGACGGTGAAGCAGATCAACATCGATT
TCGAGCGCAAGGATGCCGACCAGAACATCACGAGCTTCAAGGTGGAGCT
CAAACAAGGCGACACGTACACGAAGGTTTATCAGAAAGATACCCGCGCG
AAGCAGCAAGAAATCATTCTGCTGGATCAAGCCCAGCAAGCCAGTGCGG
TGAAAGTGACGGTTCTGAGCGCGGATGGTGGTACGATGAACTGGGTTAA
CGTGGGCATCAACGAGATCAGCGTTTACAGCGCGCCAAAAGAAACCGTG
CTGGACACGGCGGATACGAATCACATGCTGGGCGCGACGATGACCGCGA
GCAGCAACGAAACGGCGACGCTGACGCCAGATAAGGCGATCGACCAGAA
CCGCACCGGCCGCAATAATCGCTGGGCGAGTGGCTACGAGACGCCGAGT
AACATCTGGCTGAAGGCCGAATTCCCGCGCCTCACCGCGGTTAAGGACA
TCCGCATCTACTTCTTCGAGCGCGACGTTAATCCAAAACCGACGAACGT
GCAGAGTTTCGATCTGAGCTACACCGATAGCGAAGGTACGGAACACACC
CTCAAGAGCGGTTATGCGATGACGGCGAGCGGTGCGGGTTATGTTGCCG
ATGTGGTGATTCAACTCGACCAAGCCGTGAACGCCCGTAGTCTCAAGCT
GAGCAACTTCGCGATCAAAAGCAGCGAATATAATAATGTTAGCGTTGCG
GAGTGGGAGGCGTATAGTAATGATCAAGCCGAGCCGGGTGCGACGCTGG
ATAGTGTTGTTAGCGATCTGGAGAGCAACCACCTCACCATTGAAACGGA
TACGGACACGCTGGCGCTGCCAACGGTTCCAGACGGCTATACCGTGAAG
TTCAACGGCGCGGATTACGAGCAACTGATTGCCGCCGATGGCACCGTGA
ACCATCCGCTGGTGGACAAGACCGTGCAAGTTGCGTACGTTGTGACGGA
TACCGCCACCGGTAATACCAAGACCACCAGCGACATCCCATACGTGGTT
AAGGGCACGAATCAGCAGCAAGAGGGTAACAACGCCAAACCGACCATCA
TCCCGGAAATTGCGGAATGGCATAGCACCAGCGCGGCCAAGCTGGCGGC
CAGTGCCGTGACGAAGGTGGTTTACGACGACGACAGCCTCAAGGCGGTG
GTGGATGAGTTCGTGGCCGACTACAAGGATTTCACGGGCATCAAGCTGA
CGGCCAAGAAAGGCGCCGCGGAAGCCGGCGCCTTCAACTTTGTGAAAAC
CGACAGCACGGCGGCCATTGCGCAACTGGGCGATGAAGGCTACACGATG
GACATTCGCGCCGATCGCGTGGTTGCCAAAAGCAGCAGCGTGACGGGTA
ACATGTACGCCATGCAAACCATTCTGCAGATGACCAAGCAAGACGCGAA
TGGTTTCGTTATCGGCAGCATGCGTGATTACCCACGCTTCACCACGCGT
GGTCTGCTGCTGGACGTGGCGCGCAAACCAGTGAGTCTGGAAATGATGC
GTGAGATCACCCGCACCATGCGCTACTACAAGATGAACGACTTCCAAGC
CCATCTGAGTGACAATTACATTTTTCTGGAAAACTATGGCAAGGGCGAT
AACGAAGACGAGGCCTTTAAAGCCTACGATGCGTTTCGTCTGGAGAGTA
GTCTGACGAACGACAAGGGTGAAAGCCCGACCGCGGAAGACTATAGCAT
CAGTAAGAAAACGTTCAAGCAGTTTATCCAAGATGAACGCGCGCTGGGC
ATGAACGTGGTTCCAGAGATTGACGTGCCGGCGCATGCCAACAGCTTTA
CGAAGATCTGGCCGGAGCTGATGGTGAAAGGTCGCGTGAGTCCGATCAA
CAGTAACCGTCCACTGATCGATCACCTCGATGTTAGCAAGCCGGAGACG
ATCGCGAAAATCAAGGAGATCTTCGACGACTACACCAAGGGCGACGATC
CGACCTTCGACAGCGACACGACGGTTCATATTGGCGCGGAAGAGTTTCT
GTATAACTATACCGCGTATCGTAAGTTCATCAATGAAATCGTTCCATAT
ATCAAAGATACGAACACCGTGCGTATGTGGGGTGGTCTGACGTGGATCA
ATGACCACAAGACCGAGATTACGAAGGACGCGATCGAGAACGTGGAAAT
GAATCTGTGGAGTAAGGACTGGGCCGACGGTCTCCAGATGTACAATATG
GGTTATAAGCTGATTAATACGATCGACGACTACGGCTACATGGTGCCAA
ATGGCAGTTACGGCCGCGCCAACGCCTATGGTGATCTGCTCAACATCAG
CCGTGTGTTCGACAGCTTTGAGCCAAACAAGGTGCGCAGTAGCGGCGGC
TATCAAGCGGTGCCAAGTGGCGATGATCAGATGCTGGGTGCGGCCTTTG
CCATCTGGAGCGATAACATCGATAAAAGCGCCAGCGGTCTGACGGAAAG
TGATCTGTACTGGCGCTTCTTCGACGCCATGCCATTTTACGCGGAGAAA
ACGTGGGCGGCGACGGGCAAAGAGAAAGGTACCGCGGCGAAGCTGACGG
CGCTGGCGGCCAAACAAGGCACGGGCCCACGCACCAATCCGTACTACCA
AGCCACCAGCAAGAACAGTGTTTACGAGAGCTATGACTTTAACGATGGT
CTGGCCGATGCCAGTGGTAACGGTCGCGATCTCACGATTGGCGATGGCA
GTAAAGCGGCCGTGAAAGATCAGAGCCTCAAACTGGCCGGCGGTAGCAG
CTATGCCACGAGCAAGCTCGATAAACTCGGCAACGGCAACGAGCTCACC
TTTGATGTGACGCTGCAACAAGCCGCGAAACCGGGCGATATCCTCTTCG
AAGCCGACGCGCCGTACGGTACCCACGATATCCGTGTGATGGAGAACGG
CAAACTGGGCTTCACGCGCGAACTCTACAATTACTACTTTGATTACGAG
CTGCCGGTGGGCAAAACGGTTACGGTTACCATCAAGGTTGACCAGCAGA
CCACCAAACTGTATGTGGACGGCGAATTCGTGAGTGACGCGACCGGCAA
GTACATCGACAAGGGCATCGAGAAGAAAACGGGTATTACCGCCGCCACC
TTTGCGCTCCCGCTGCAGCGTATCGGCAGTAAGACCAGCGCCATCAATG
GCGTGATTGACAATGTGATTGTGAAGAAGAGCGAGGCCGAAACCGACCA
GTACAACAAGAGTTGTTGGACCGGCACGACCAACAGCGAGACGCAATAC
AACGATACCGAGGGTCTGCTGCGCTACGCCTTCGACAACAATCCGAGTA
CCATCTGGCACAGCAATTGGAAAGGTGCGACCGATAAGCTGACGGGCAG
TAACAGCTTTTATGCGGAAATTGACATGTGTCAGAAATACACGATTAAC
CAGTTCAGCTTTACGCCGCGTACCAGCCAAGATAGCGGCCAAGTGACGA
AAGCCGATCTGTATGTGAAGGCCAATGCGAACGACGAATGGAAACAAGT
TGCCACCGATCAAGTGTTTGAGGCGAGTCGCGCCAAAAAGACGTTTATG
TTCGACGAGCAAGAAGTGCGTTACGTGAAATTCGTGGCCAAGAGCAGTA
ACGATGGTTGGGTTGCCGTGAGTGAGTTCGGCGTGGCCAATAAGCCGAG
CAGTACCGTTCGCGTTTTTGTTGCCGCGGATCCAGCGGAGGGTGGTACC
GTTAGTGTTGCCGCCGAAGGCGAAACGGGTACCGATACGGCGGTGGACG
TTGCCAGCGGTGCCAGTGTTACCGCGAAAGCGGTGGCGGCGGACGGCTA
TCGCTTCAGTGGTTGGTTTACGACCGCGAGTGAAACCGCCGTGAGCACC
GACGCGACCTATACCTTCGCGGCCGATGGTAATACCGCGCTGACCGCGA
AATTCACCAAAGATAGCACCCCAGATCCGGGCCCGAAGCCAACCATCAG
TAGTATCGCGGTGACGAAGCCAACCGTGACCGATTACAAAGTGGGCGAT
ACCTTCGACGCGACCGGTCTCGCCGTGACCGCCACGATGAGCGATGGCA
GCACGAAGACGCTGACCGCCGGTGAGTATACGCTCAGCGCCACCCAAGA
TGGCGCGGCCGTTGCGCTCGACAAAGCCTTTGCCAAAGCCGGTAAGGTT
ACGGTGACGGTGACGGCCAATGGTAAAACGGCCACCTTCGACGTGACCG
TTACGGCGAAAGACCCAGACCCGGAACCGGCGACGCTGAAAAGCATCAA
GGTGACCAGCAAACCGGATAAGACCACGTACACGGTTGACGAAACCTTC
GCCAAAACCGGTCTGGCCGTTACCGGTACGTGGAGTGATGGTAAGACCG
CGCTGCTCAAGGATGGTGAATACAAGCTCAGCGCGGTTGACGCCGACGG
CAAGACGGTTGATCTCACGAAGCCGTTCACGGCCGCGGGTGATGTGACG
GTGACCGTTACCAGCGGTAAGCTGACCGACAGCTTTACCATCACCGTGA
AAGCCAAAACCGTGACCCCGGCCCCGGGTGATAATAAGCCGGGTGAGAA
CAAGCCGGGTGCCGACAAGCCAAAGCCGAATACCCCAGACGAAGTTGCC
AAAGGTGGTGGCGGTAGCCACCATCACCATCACCACTAA
MGSDDNLALNQTVTASSYEVATTAPEKAVDGDLGTRWGTAQNKAANEWI
EVGLGGTKTVKQINIDFERKDADQNITSFKVELKQGDTYTKVYQKDTRA
KQQEIILLDQAQQASAVKVTVLSADGGTMNWVNVGINEISVYSAPKETV
LDTADTNHMLGATMTASSNETATLTPDKAIDQNRTGRNNRWASGYETPS
NIWLKAEFPRLTAVKDIRIYFFERDVNPKPTNVQSFDLSYTDSEGTEHT
LKSGYAMTASGAGYVADVVIQLDQAVNARSLKLSNFAIKSSEYNNVSVA
EWEAYSNDQAEPGATLDSVVSDLESNHLTIETDTDTLALPTVPDGYTVK
FNGADYEQLIAADGTVNHPLVDKTVQVAYVVTDTATGNTKTTSDIPYVV
KGTNQQQEGNNAKPTIIPEIAEWHSTSAAKLAASAVTKVVYDDDSLKAV
VDEFVADYKDFTGIKLTAKKGAAEAGAFNFVKTDSTAAIAQLGDEGYTM
DIRADRVVAKSSSVTGNMYAMQTILQMTKQDANGFVIGSMRDYPRFTTR
GLLLDVARKPVSLEMMREITRTMRYYKMNDFQAHLSDNYIFLENYGKGD
NEDEAFKAYDAFRLESSLTNDKGESPTAEDYSISKKTFKQFIQDERALG
MNVVPEIDVPAHANSFTKIWPELMVKGRVSPINSNRPLIDHLDVSKPET
IAKIKEIFDDYTKGDDPTFDSDTTVHIGAEEFLYNYTAYRKFINEIVPY
IKDTNTVRMWGGLTWINDHKTEITKDAIENVEMNLWSKDWADGLQMYNM
GYKLINTIDDYGYMVPNGSYGRANAYGDLLNISRVFDSFEPNKVRSSGG
YQAVPSGDDQMLGAAFAIWSDNIDKSASGLTESDLYWRFFDAMPFYAEK
TWAATGKEKGTAAKLTALAAKQGTGPRTNPYYQATSKNSVYESYDFNDG
LADASGNGRDLTIGDGSKAAVKDQSLKLAGGSSYATSKLDKLGNGNELT
FDVTLQQAAKPGDILFEADAPYGTHDIRVMENGKLGFTRELYNYYFDYE
LPVGKTVTVTIKVDQQTTKLYVDGEFVSDATGKYIDKGIEKKTGITAAT
FALPLQRIGSKTSAINGVIDNVIVKKSEAETDQYNKSCWTGTTNSETQY
NDTEGLLRYAFDNNPSTIWHSNWKGATDKLTGSNSFYAEIDMCQKYTIN
QFSFTPRTSQDSGQVTKADLYVKANANDEWKQVATDQVFEASRAKKTFM
FDEQEVRYVKFVAKSSNDGWVAVSEFGVANKPSSTVRVFVAADPAEGGT
VSVAAEGETGTDTAVDVASGASVTAKAVAADGYRFSGWFTTASETAVST
DATYTFAADGNTALTAKFTKDSTPDPGPKPTISSIAVTKPTVTDYKVGD
TFDATGLAVTATMSDGSTKTLTAGEYTLSATQDGAAVALDKAFAKAGKV
TVTVTANGKTATFDVTVTAKDPDPEPATLKSIKVTSKPDKTTYTVDETF
AKTGLAVTGTWSDGKTALLKDGEYKLSAVDADGKTVDLTKPFTAAGDVT
VTVTSGKLTDSFTITVKAKTVTPAPGDNKPGENKPGADKPKPNTPDEVA
KGGGGSHHHHHH*

Method

Expression: TB media, 0.5 mM IPTG, 20 hrs, 25° C.

Lysis Buffer: 20 mM Tris, pH 7.5, 500 mM NaCl, 20 mM imidazole

Affinity Chromatography:
a) Loading of spun down lysate.
b) Gradient: 20 mM Tris, pH 7.5, 500 mM NaCl, 20-500 mM imidazole, 10CV

Dialysis: Final buffer formulation: 20 mM Tris pH 7.5, 10% glycerol

Final yield: 278 mg from 4L expression(fermentation), 2.78 mg/ml

### Example 5: Semi-Continuous Production of LNTII

LNTII is produced via the combination of two continuous stirred-tank reactors (CSTRs) to produce N-acetylglucosamine (GlcNAc-oxa) and a packed-bed reactor (Scaffolded BNC) that converts GlcNAc-oxa together with added lactose to Lacto-N-triose II (LNTII). The first CSTR converts GlcNAc and DMC (2-Chloro-1,3-dimethylimidazolinium chloride) in triethylamine (Et₃N) at 0° C. to GlcNAc-oxa. This reaction product can then be fed semi-continuously or continuously to an intermediate CSTR, along with buffered lactose, then fed into the packed-bed reactor (Scaffolded BNC) comprised of immobilized Bbh1 (system module 6, SM6 consisting of elementary module EM2.2A; see FIG. 1D and FIG. 2A).

Scaffolded BNC setup and operation: 125 g of S40W is scaffolded with 2.5 g of magnetite nanoparticles (2% w/w) as described (“Enzyme Immobilization Method and Protocol”) onto which enzyme (Bbh1) stock solution (250 mg Bbh1) is added. The immobilization yield is determined by Bradford analysis. The Bbh1 immobilized scaffold (scaffold-MNP complex) is packed into a column (ID 1.0″×length 4.0″; packed bed volume=50 ml).

CSTR setup and operation: GlcNAc-oxa is produced by pumping 0.5M GlcNAc (100 ml/hr) and 7.0M Et₃N (8.9 ml/hr; steady state concentration in CSTR=0.625M) into a jacketed CSTR cooled to 0° C. with a chiller (FIG. 10). Solid DMC (25.4 g/hr; steady state concentration in CSTR=1.5M) is simultaneously pumped into the reactor under agitation with a residence time of 0.5 h. The pH is maintained at 9.5<pH<11.0 with a basic feed controlled by a pH controller (pHC). The product stream containing GlcNAc-oxa is then pumped to an intermediate jacketed CSTR under agitation. 1.0M Tris buffer at pH 7.5 (10 ml/hr; steady state concentration in CSTR=0.1 M) and solid lactose (7.53 g/hr; steady state concentration in CSTR=0.22 M) are simultaneously pumped into the intermediate jacketed CSTR, which is also cooled to 0° C. The pH of the reaction mixture is maintained at 7.5 by pumping in HCl (5 M) and using a pH controller. The production rate of GlcNAc-oxa is set at or slightly above the flow rate of the packed-bed reactor (100 ml/hr=2 BV/hr; 1) while retaining a minimal volume of 20 ml in the intermediate CSTR.

CSTR and packed-bed reactor operation: The solution from the intermediate reactor is pumped into the packed bed reactor using a peristaltic pump-(Cole Partner Masterflex L/S 7522-30 Pump W/ Masterflex Easy Load 3 Head) at a flow rate of 100.0 ml/hr and 10 ml fractions were collected and analyzed by HPLC (System: Thermo Scientific Vanquish Duo; Column: Acclaim Trinity P1; CAD detection; Solvent A: Acetonitrile; solvent B: 20 mM ammonium formate pH 4.45. 0-2 min: 22% of B; 2-4 min: 22-35% of B; 4-5.5 min: 35% of B; 5.5-8 min: 35-70% of B; 8-10.5 min 70% of B; 10.5-13.5 min: 70-22% of B; 13.5-15 min: 22% of B.

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

1. A module comprising a magnetic macroporous matrix material comprising self-assembled mesoporous aggregates of magnetic nanoparticles magnetically entrapping an immobilized glycan synthesis enzyme.

2. The module according to claim 1 wherein said self-assembled mesoporous aggregates comprise a single glycan synthesis enzyme or more than one glycan synthesis enzyme.

3. The module according to claim 2, wherein said glycan synthesis enzyme is a sugar activation enzyme, a sugar extension enzyme, a reagent regeneration enzyme, a sugar functionalization enzyme, a sugar support enzyme, or a sugar removal enzyme.

4. The module according to claim 3, wherein said glycan synthesis enzyme is a sugar activation enzyme EM-1.

5. The module according to claim 3, wherein said glycan synthesis enzyme is a sugar extension enzyme EM-2.

6. The module according to claim 3, wherein said glycan synthesis enzyme is a reagent regeneration enzyme EM-3.

7. The module according to claim 3, wherein said glycan synthesis enzyme is a functionalization enzyme EM-4.

8. The module according to claim 3, wherein said glycan synthesis enzyme is a support enzyme module EM-5.

9. The module according to claim 3, wherein said glycan synthesis enzyme is a sugar removal module EM-6.

10.-35. (canceled)

36. A system module, comprising a module according to claim 3.

37.-40. (canceled)

41. A method of making a glycan, comprising modifying a glycan substrate with a module according to claim 3, wherein said module modifies a glycan substrate to produce a glycan.

42. A method for making a glycan, comprising the steps of
preparing a scaffolded bionanocatalyst comprising a glycan synthesis enzyme;
contacting the scaffolded bionanocatalyst with a glycan substrate;
converting the glycan substrate into a glycan.

43.-54. (canceled)

55. A device for producing a glycan, comprising a module or a system module according to claim 3, wherein said module modifies a glycan substrate to produce a glycan.

56. A device for producing a glycan, comprising a first module according to claim 3 and a second module, according to claim 3 wherein a first substrate is introduced into said first module to create a modified first substrate;
wherein said first modified substrate is a second substrate introduced into said second module to produce a glycan.

57.-60. (canceled)

61. A method of making a device for catalyzing an enzymatic reaction, comprising combining a magnetic macroporous scaffold with self-assembled mesoporous aggregates of magnetic nanoparticles and a glycan synthesis enzyme, wherein said enzyme is magnetically immobilized within or in said mesopores.

62. A modular process for producing a glycan, comprising a module wherein: said module comprises a magnetic macroporous powder comprising magnetic microparticles, wherein said powder has immobilized a preparation of self-assembled mesoporous aggregates of magnetic nanoparticles containing a glycan synthesis enzyme; wherein a substrate is introduced into said module (or passed through a flow cell) and said substrate is modified to provide a glycan.

63. A process for preparing a scaffolded bionanocatalyst by combining a magnetic nanoparticle and a glycan synthesis enzyme to form a bionanocatalyst and then contacting the bionanocatalyst with a scaffold to obtain the scaffolded bionanocatalyst.

64. A process for preparing a scaffolded bionanocatalyst by combining a scaffold and a magnetic nanoparticle to form a scaffolded magnetic nanoparticle complex and then contacting the scaffolded magnetic nanoparticle complex with a glycan synthesis enzyme.

65. A scaffolded bionanocatalyst made by the process of claim 64.

66. A scaffolded bionanocatalyst comprising a magnetic macroporous matrix material comprising self-assembled mesoporous aggregates of magnetic nanoparticles magnetically entrapping an immobilized glycan synthesis enzyme.

67.-68. (canceled)

69. A method of catalyzing a reaction between a plurality of substrates, comprising exposing a scaffolded bionanocatalyst according to claim 66 to said substrates under conditions in which said scaffolded bionanocatalyst catalyzes said reaction between said substrates.

70. A method of catalyzing a reaction between a plurality of glycan substrates, comprising exposing the glycan substrates to a module according to claim 3 to said substrates under conditions in which said module catalyzes said reaction between said substrates.

71.-73. (canceled)
